(19) United States
(12) Patent Application Publication
(10) Pub. No.: US 2025/0276018 A1
(43) Pub. Date: Sep. 4, 2025

(54) CAR CONSTRUCTS AND METHODS OF TREATMENT

(71) Applicant: AffyImmune Therapeutics Inc., Natick, MA (US)

(72) Inventor: Alyssa Birt, Northborough, MA (US)

(21) Appl. No.: 19/002,602

(22) Filed: Dec. 26, 2024

Related U.S. Application Data
(60) Provisional application No. 63/616,278, filed on Dec. 29, 2023.

(51) Int. Cl. A61K 35/17; A61K 31/506; A61K 40/31; A61K 40/42; A61K 45/06; A61K 51/12; A61P 35/00; C07K 14/655; C07K 14/705; C07K 14/725; C07K 16/28; C12N 15/86

(52) U.S. Cl. CPC A61K 35/17; A61K 31/506; A61K 40/31; A61K 40/421; A61K 40/4237; A61K 45/06; A61K 51/1203; A61P 35/00; C07K 14/655; C07K 14/7051; C07K 14/70521; C07K 14/70578; C07K 16/2818; C07K 16/2821; C07K 16/2827; C12N 15/86; A61K 2123/00; A61K 2239/13; A61K 2239/17; A61K 2239/21; A61K 2239/31; A61K 2239/38; C07K 2317/565; C07K 2317/622

(57) ABSTRACT

The present disclosure relates to low affinity chimeric antigen receptors (CARs) and CAR-T cells, which provide cytotoxicity against tumors overexpressing the proto-oncogene c-MET and alleviate on-target, off-tumor toxicities. The CAR-T cells of the present disclosure comprise low affinity anti-c-MET scFvs, which facilitate enhanced anti-tumor activity and a reduced rate of tumor relapse.

### CROSS-REFERENCE TO RELATED APPLICATIONS

This application claims priority to U.S. Provisional Patent Application No. 63/616,278, filed on Dec. 29, 2023, the entire content of which is incorporated herein by reference.

### SEQUENCE LISTING

The instant application contains a Sequence Listing which has been submitted electronically in XML file format and is hereby incorporated by reference in its entirety. Said XML copy, created on Jan. 20, 2025, is named 115568-0030_SL.xml and is 246,882 bytes in size.

### FIELD

The present disclosure relates to novel c-MET CAR constructs, c-MET CAR-expressing cells and methods of their use in the treatment of cancer.

### BACKGROUND

Adoptive cell transfer therapy is a type of immunotherapy involving ex vivo expansion of autologous or allogeneic immune cells and subsequent infusion into a patient. The immune cells may be modified ex vivo to specifically target malignant cells. Modifications include engineering of T cells to express chimeric antigen receptors (CARs). The advantage of CAR-T technology compared with chemotherapy or therapeutic antibodies is that reprogrammed engineered T cells can proliferate and persist in the patient and work like a living drug.

The ideal CAR target antigen would be a native, surface-exposed tumor neoantigen that is highly expressed and is undetectable in healthy tissues. However, many commonly targeted solid tumor antigens are also expressed by non-tumor tissues, albeit at lower levels. CAR molecules with high affinity for such antigens can lead to collateral targeting of healthy tissues resulting in on-target, off-tumor toxicity, a major limiting factor to the progress of CAR T cell therapy to date.

C-MET is a member of the receptor tyrosine kinase family of proteins, and the product of the proto-oncogene MET. See, e.g., https://www.ncbi.nlm.nih.gov/gene/4233. The encoded preproprotein is proteolytically processed to generate alpha and beta subunits that are linked via disulfide bonds to form the mature receptor. Binding of its ligand, hepatocyte growth factor (HGF), induces dimerization and activation of the receptor, which plays a role in cellular survival, embryogenesis, and cellular migration and invasion. Id. Amplification and overexpression of this gene are associated with multiple human cancers. As a proto-oncogene, abnormal activation of c-MET can promote the development and progression of multiple carcinomas such as liver, lung, colon, rectal, breast, pancreatic, ovarian, prostate, renal hepatocellular, gastric, and various head and neck carcinomas, in addition to cancers of the nervous system such as glioblastoma.

High affinity c-MET CAR-T cells recognize c-MET-expressing cells, including both normal epithelial tissues with low levels of c-MET and carcinomas with considerably higher c-MET expression levels. The recognition of antigen both on normal, non-target cells as well as on cancer cells can lead to severe toxicities and T cell exhaustion. CAR-T cell exhaustion has been recognized as a major cause of nonresponse and relapse associated with CAR-T cell therapy. T cells expressing chimeric antigen receptors (CARs) at high levels undergo tonic, antigen independent signaling due to receptor clustering, and function poorly as a result of T cell exhaustion, as evidenced by high levels of PD-1, TIM-3, LAG-3 expression, diminished antigen induced cytokine production, poor CAR-T cell persistence, and excessive programmed cell death. These problems are particularly acute in CAR-T treatments in patients with solid tumors, particularly carcinomas.

It is therefore of great interest to develop CARs and CAR-T expressing cells facilitating more selective killing of tumor cells, reduced T cell exhaustion and T cell activation, improved in vivo persistence, and reduced systemic toxicities.

### SUMMARY OF THE INVENTION

Previously, it was observed that Met is expressed in normal human tissue (liver, gastric, thyroid and kidney) and neoplastic human tissues (Di Renzio et al. (1991) 6(11):1997-2003). The inventors observed that CAR-T cells expressing anti-c-MET scFvs have the undesirable effect of killing healthy cells and tissue. Accordingly, it has now been unexpectedly found that affinity tuned anti-c-MET scFvs described herein avoid this problem and exhibit reduced killing of healthy cells (lung, liver, colon, and kidney epithelial) while maintaining efficacy of high c-met expressing cancer cell lines. The present disclosure is based, at least in part, on the development of affinity tuned chimeric antigen receptors (CARs) with variant heavy chain variable (V_{H}) region CDR3 domains providing reduced binding affinity to c-MET as compared to a corresponding binding domain from the parent antibody.

Accordingly, one aspect of the present disclosure features a chimeric antigen receptor (CAR) comprising: (a) an affinity-tuned extracellular antigen binding domain; (b) a co-stimulatory domain; and an (c) activation domain. The affinity-tuned extracellular antigen binding domain comprises an anti-c-MET antibody. In some embodiments, the anti-c-MET antibody is a single-chain antibody fragment (scFv).

In some embodiments, the extracellular binding domain comprises a heavy chain variable (V_{H}) region comprising complementary determining 1 (HC CDR1), complementary determining 2 (HC CDR2), and complementary determining 3 (HC CDR3) regions; and a light chain variable (V_{L}) region comprising complementary determining 1 (LC CDR1), complementary determining 2 (LC CDR2), and complementary determining 3 (LC CDR3) regions, wherein: the V_{H} region comprises HC CDR1 and HC CDR2 regions comprising amino acid sequences set forth SEQ ID NOs: 1 and 2, respectively, and an HC CDR3 region comprising an amino acid sequence set forth in any one of SEQ ID NOs: 3-8; and the V_{L} region comprises LC CDR1, LC CDR2, and LC CDR3 regions comprising amino acid sequences set forth in SEQ ID NOs: 9-11, respectively. In certain embodiments, the HC CDR3 comprises an amino acid sequence set forth in SEQ ID NO: 5 or SEQ ID NO: 8.

In some embodiments, the V_{H} and V_{L} region amino acid sequences are selected from: (a) SEQ ID NOs: 13 and 19, respectively; (b) SEQ ID NOs: 14 and 19, respectively; (c) SEQ ID NOs: 15 and 19, respectively; (d) SEQ ID NOs: 16 and 19, respectively; (e) SEQ ID NOs: 17 and 19, respectively; or (f) SEQ ID NOs: 18 and 19, respectively. In some embodiments, the extracellular binding domain comprises an anti-c-MET scFv comprising an amino acid sequence set forth in any one of SEQ ID NOs: 29-34.

In some embodiments, the V_{H} region in the extracellular binding domain (e.g., anti-c-MET single-chain antibody) comprises HC CDR1 and HC CDR2 regions comprising the amino acid sequences set forth in SEQ ID NOs: 70 and 71, respectively, and an HC CDR3 region comprising an amino acid sequence set forth in any one of SEQ ID NOs: 72-75; and the V_{L} region comprises LC CDR1, LC CDR2, and LC CDR3 regions comprising amino acid sequences set forth in SEQ ID NOs: 76-78, respectively.

In some embodiments, the extracellular binding domain comprises V_{H} and V_{L} region amino acid sequences selected from: (a) SEQ ID NOs: 80 and 84, respectively; (b) SEQ ID NOs: 81 and 84, respectively; (c) SEQ ID NOs: 82 and 84, respectively; or (d) SEQ ID NOs: 83 and 84, respectively. In some embodiments, extracellular binding domain comprises an anti-c-MET scFv comprising an amino acid sequence set forth in any one of SEQ ID NOs: 92-95.

In some embodiments, the anti-c-MET scFv comprises a linker between the V_{H} and V_{L} regions. In some embodiments, the linker comprises the amino acid sequence (GxS)n in which x is an integer between 1-6, inclusive, and n is an integer between 1-10, inclusive. In some embodiments, the linker comprises the amino acid sequence (GGGS)n in which n is an integer between 1-10, inclusive (SEQ ID NO: 143). In one embodiment, the linker comprises the amino acid sequence multimer GGGGSGGGGSGGGGS (SEQ ID NO: 121).

In some embodiments, the costimulatory domain is from 4-1BB (CD 137), CD28, OX40, ICOS, GITR, CD27, CD30, CD40, DAP 10, DAP12, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD 160, B7-H3, a ligand that specifically binds with CD83, or a combination thereof. In one embodiment, the extracellular antigen binding domain (or scFv) comprises a costimulatory domain from 4-1BB (SEQ ID NO: 122), CD28 (SEQ ID NO: 123) or OX-40 (SEQ ID NO: 124). In another embodiment, the extracellular binding domain (or scFv) comprises costimulatory domains from 4-1BB (SEQ ID NO: 122) and CD28 (SEQ ID NO: 123). In some embodiments, the costimulatory domain comprises a mutant CD28 costimulatory domain as described in U.S. Patent Application No. 2020/0129554.

In some embodiments, the activation domain comprises one or more immunoreceptor tyrosine-based activation motifs (ITAMs). Examples of ITAM containing primary activation domain include, but are not limited to, those derived from CD3 zeta, common FcR gamma (FCER1G), Fc gamma RIIa, FcR beta (Fc epsilon lb), CD3 gamma, CD3 delta, CD3 epsilon, CD5, CD22, CD79a, CD79b, CD278 (ICOS), FcεRI, and CD66d. In one embodiment, the activation domain is from CD3 zeta (SEQ ID NO: 125).

In some embodiments, the CAR further comprises a hinge domain, a transmembrane domain, or a combination thereof, which is located between the extracellular binding domain and the costimulatory domain. In some embodiments, the CAR disclosed herein may further comprise a hinge domain, which may be linked to the C-terminus of the extracellular antigen binding domain and to the N-terminus of the transmembrane domain. In some embodiments, the CAR comprises a hinge domain from CD8 alpha (SEQ ID NO: 126), CD28 (SEQ ID NO: 127), or IgG4 (SEQ ID NO: 128).

In some embodiments, the CAR comprises a transmembrane domain from a cell surface receptor, which is C-terminal to the extracellular antigen binding domain and N-terminal to the costimulatory domain. Exemplary transmembrane domains, include but are not limited to those derived from an alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, CD271, CD278 (ICOS), TNFRSF18 (GITR), TNFRSF19, or killer cell immunoglobulin-like receptor (KIR). In certain embodiments, the transmembrane domain is from CD8 alpha (SEQ ID NO: 129), CD28 (SEQ ID NO: 130), ICOS (SEQ ID NO: 131), or GITR (SEQ ID NO: 132).

In exemplary embodiments, the c-MET CAR comprises an amino acid sequence set forth in any one of SEQ ID NOs: 43-48 or 102-105.

In some embodiments, the c-MET CAR is a bispecific CAR or a tandem CAR (tanCAR), which further comprises a second extracellular antigen binding domain. In some embodiments, the second extracellular antigen binding domain is a second scFv. In some embodiments, the second extracellular binding domain is a binding domain of a protein ligand. In some embodiments, the second extracellular antigen binding domain comprises an αL subunit I domain of human lymphocyte function-associated antigen-1 (LFA-1) targeting ICAM-1. In one embodiment, the binding domain comprises an amino acid sequence set forth in SEQ ID NO: 134 or a mutant thereof. In some embodiments, the second extracellular binding domain targets a tumor-associated antigen (TAA), particularly one that is overexpressed in solid tumors and metastatic tumors.

Any of the CARs disclosed herein may further comprise a signal peptide located at the N-terminus of the CAR precursor protein. This may include, for example, a CD8 alpha chain signal peptide (SEQ ID NO: 162).

In another aspect, the present disclosure provides nucleic acids encoding the c-MET scFvs and CARs described herein. In some embodiments, the nucleic acid encoding an anti-c-MET scFv comprises a nucleotide sequence set forth in any one of SEQ ID NOs: 36-41 or 92-95. In some embodiments, the nucleic acid encoding a c-MET CAR comprises a nucleotide sequence set forth in any one of SEQ ID NOs: 57-62, 64-69, 112-115, or 117-120. In some embodiments, the nucleic acid is a plasmid. In some embodiments, the nucleic acid is a vector, such as a lentivirus vector. In some embodiments, the nucleic acid is an RNA.

In some embodiments, the nucleic acid further comprises a human somatostatin receptor 2 (SSTR2) coding sequence. In some embodiments, the nucleic acid encodes a full length SSTR2 polypeptide (aa 1-381; SEQ ID NO: 137). In other embodiments, the nucleic acid encodes a truncated SSTR2 polypeptide (aa 1-314; SEQ ID NO: 136). In certain embodiments, the nucleic acid encodes an SSTR2-CAR fusion protein comprising an amino acid cleavage sequence between the SSTR2 and CAR coding sequences. In some embodiments, the SSTR2 coding sequence is 5′ of the CAR coding sequence. In other embodiments, the CAR coding sequence is 5′ of the SSTR2 coding sequence. In some embodiments, the amino acid cleavage sequence is encoded by a self-cleaving 2A peptide sequence from porcine teschovirus-1 (P2A), equine rhinitis A virus (E2A), Thosea asigna virus (T2A), or foot-and-mouth disease virus (F2A), or a combination thereof. In certain embodiments, the amino acid cleavage sequence comprises an amino acid sequence set forth in any one of SEQ ID NOs: 139-142.

In another aspect, the present disclosure provides a population of immune cells expressing one or more of the c-MET CARs disclosed herein. In some embodiments, the population of cells comprises one or more of the nucleic acids encoding an anti-c-MET CAR and/or an additional nucleic acid described herein. In some embodiments, the population of c-MET expressing cells further expresses a human somatostatin receptor 2 (SSTR2) marker protein. In some embodiments, the population of cells are immune cells. In some embodiments, the immune cells are T cells, natural killer (NK) cells, tumor infiltrating lymphocytes, dendritic cells, macrophages, B cells, neutrophils, eosinophils, basophils, mast cells, myeloid-derived suppressor cells, stem cells, precursors thereof, subtypes thereof, or a combination thereof; optionally wherein the immune cell is a human immune cell. In some embodiments, the immune cells are T cells. In some embodiments, the T cells additionally express an SSTR2 marker. In some embodiments, the population of cells further comprises a second population of immune cells. In some embodiments, the second population of immune cells expresses a second CAR or other polypeptide of interest.

In another aspect, the present disclosure provides a cell therapy-based method of treating cancer comprising administering to a subject in need thereof a population of immune cells described herein. In some embodiments, the subject is a human patient. In some embodiments, the cancer is a solid tumor. In some embodiments, the solid tumor is a carcinoma. In some embodiments, the carcinoma is a small cell lung carcinoma, non-small cell lung carcinoma, squamous cell lung carcinoma, large cell lung carcinoma, pancreatic carcinoma, pancreatic ductal carcinoma, prostate carcinoma, esophageal carcinoma, breast carcinoma, ovarian carcinoma, prostate carcinoma, colorectal carcinoma, bladder carcinoma, cervical carcinoma, hepatocellular carcinoma, renal hepatocellular carcinoma, gastric carcinoma, papillary carcinoma, adrenocortical carcinoma, pituitary carcinoma, a head and neck carcinoma, an adenocarcinoma thereof, or a squamous cell carcinoma thereof. In some embodiments, the cancer is a glioblastoma or mesothelioma. In some embodiments, the cancer is metastatic.

In some embodiments, the method further comprises administering the cell therapy to a human patient prior to, concurrently, or after a therapy against the cancer to reduce tumor burden. In some embodiments, the therapy is a chemotherapy, an immunotherapy, a radiotherapy, or a surgery. In some embodiments, prior to the cell therapy, the subject has been administered a lymphodepleting treatment to condition the subject for the cell therapy. In some embodiments, the lymphodepleting treatment comprises administering to the subject fludarabine and/or cyclophosphamide.

In some embodiments, the therapy comprises administration of an immune checkpoint inhibitor, such as pembrolizumab (Keytruda™), ipilimumab (Yervoy™) nivolumab (Opdivo™), or atezolizumab (Tecentriq™). In some embodiments, the cell therapy comprises administration of a therapeutic antibody selected from the group consisting of abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomab, ocaratuzumab, ofatumumab, obinutuzumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8.

In some embodiments, the method of cell therapy further comprises administration of a tyrosine kinase inhibitor capable of inhibiting TCR signaling and/or CAR signaling, such as dasatinib, ponatinib, saracatinib, bosutinib, nilotinib, or a combination thereof. In a particular embodiment, the inhibitor is dasatinib. In some embodiments, the tyrosine kinase inhibitor is administered for a period of time sufficient to restore at least partial T cell function and then discontinued. In some embodiments, the tyrosine kinase inhibitor is administered continuously. In other embodiments, the tyrosine kinase inhibitor is administered intermittently. In some embodiments, the tyrosine kinase inhibitor is administered intermittently to facilitate periods of T cell inactivation (e.g., during pharmaceutical composition administration) and periods of T cell activation (e.g., during absence of pharmaceutical composition administration). In some embodiments, the tyrosine kinase inhibitor is administered intermittently so that the concentration is maintained below a threshold level required to block CAR-T cell function.

In another aspect, the present disclosure provides a method for treating a cancer and monitoring CAR-T cell distribution in a patient. The method comprises the steps of: (a) incubating a population of CAR-T cells expressing SSTR2 with a radioactive label that binds to SSTR2; (b) intravenously infusing the labeled CAR-T cells into a patient in an amount of 10⁶-10⁸ cells/kg patient; and detecting the labeled CAR-T cell distribution by PET/CT imaging, wherein the labeled CAR-T cells are infiltrated into cancer cells to kill the cancer cells. In some embodiments, the label is radioactively labeled DOTATOC or radioactively labeled DOTATATE. In certain embodiments, DOTATOC or DOTATATE is radiolabeled with ⁶⁸Ga. In certain embodiments, the cancer is thyroid cancer, gastric cancer, pancreatic cancer, or breast cancer.

In another embodiment, a method for treating cancer and monitoring CAR-T cell distribution in a patient, comprises the steps of: (a) intravenously a population of CAR-T cells expressing SSTR2 into a patient, where the CAR-T cells have been transduced to express at least 100,000 molecules of SSTR2 per T cell; (b) injecting into the patient a radioactive label that binds to SSTR2 at least one hour prior to PET/CT imaging, and (c) detecting the labeled CAR-T cell distribution by PET/CT imaging, wherein the labeled CAR-T cells are infiltrated into cancer cells to kill the cancer cells. In certain embodiments, the cancer is thyroid cancer, gastric cancer, pancreatic cancer, or breast cancer.

In another aspect, the present disclosure provides method of producing a population of genetically engineered immune cells. The method comprises introducing into a population of immune cells a nucleic acid coding for a CAR disclosed herein, and optionally one or more exogenous nucleic acids. In some embodiments, the immune cells include T cells, natural killer (NK) cells, tumor infiltrating lymphocytes, dendritic cells, macrographs, B cells, neutrophils, eosinophils, basophils, mast cells, myeloid-derived suppressor cells, stem cells, precursors thereof, or a combination thereof; optionally wherein the immune cell is a human immune cell. In some embodiments, the population of genetically engineered immune cells are CAR-T cells. In some embodiments, the method further comprises the step of expanding a population of CAR-T cells in the presence of a tyrosine kinase inhibitor capable of inhibiting TCR signaling and/or CAR signaling. In some embodiments, the tyrosine kinase is dasatinib. In other embodiments, the tyrosine kinase is ponatinib, saracatinib, bosutinib, nilotinib, or a combination thereof.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 is a schematic depiction of a CAR expression construct used in the Examples described herein.

FIG. 2 depicts binding curves reflecting the binding of c-MET to parent Affy7 CAR-T-cells and to two affinity-reduced Affy7 variant CAR-T cells, including the results of this analysis expressed in EC50 and fold change in binding relative to the parent Affy7 CAR-T cells.

FIG. 3 depicts binding curves reflecting the binding of c-MET to parent Affy7 CAR-T-cells and to four affinity-reduced Affy7 variant CAR-T cells, including the results of this analysis expressed in EC50 and fold change in binding relative to the parent Affy7 CAR-T cells.

FIG. 4 depicts binding curves reflecting the binding of c-MET to parent Affy6 CAR-T-cells and to two affinity-reduced Affy6 variant CAR-T cells, including the results of this analysis expressed in EC50 and fold change in binding relative to the parent Affy6 CAR-T cells.

FIG. 5 depicts binding curves reflecting the binding of c-MET to parent Affy6 CAR-T-cells and to four affinity-reduced Affy6 variant CAR-T cells, including the results of this analysis expressed in EC50 and fold change in binding relative to the parent Affy6 CAR-T cells.

FIG. 6 depicts in vitro cytotoxicity of Affy7 parent CAR-T cells and five affinity-reduced variant Affy7 CAR-T cell populations against an SNU-638 human stomach carcinoma cell line.

FIG. 7A depicts in vitro cytotoxicity of Affy7 parent CAR-T cells and five affinity-reduced Affy7 variant CAR-T cell populations against an Hs746T human stomach carcinoma cell line. FIG. 7B shows expression of c-met on the cell surface of HS746T cells.

FIG. 8 depicts in vitro cytotoxicity of Affy7 parent CAR-T cells and five affinity-reduced Affy7 variant CAR-T cell populations against an H1993 human non-small cell lung adenocarcinoma cell line.

FIG. 9 depicts in vitro cytotoxicity of Affy6 parent CAR-T cells and four affinity-reduced Affy6 variant CAR-T cell populations against an SNU-638 human stomach carcinoma cell line.

FIGS. 10A-10D show expression levels of c-MET as determined by flow cytometry in normal, healthy human epithelial cells from lung (10A), liver (10B), colon (10C), and kidney (10D).

FIG. 11, panels A and B depict in vitro cytotoxicity of Affy7 parent CAR-T cells and seven affinity-reduced Affy7 variant CAR-T cell populations against primary human hung cells.

FIG. 12, panels A and B depict in vitro cytotoxicity of Affy6 parent CAR-T cells and five affinity-reduced Affy6 variant CAR-T cell populations against primary human hung cells.

FIG. 13, panels A and B depict in vitro cytotoxicity of Affy7 parent CAR-T cells and seven affinity-reduced Affy7 variant CAR-T cell populations against primary human liver cells.

FIG. 14, panels A and B depict in vitro cytotoxicity of Affy6 parent CAR-T cells and five affinity-reduced Affy6 variant CAR-T cell populations against primary human liver cells.

FIG. 15, panels A and B depict in vitro cytotoxicity of Affy7 parent CAR-T cells and seven affinity-reduced Affy7 variant CAR-T cell populations against primary human kidney cells.

FIG. 16, panels A and B depict in vitro cytotoxicity of Affy6 parent CAR-T cells and five affinity-reduced Affy6 variant CAR-T cell populations against primary human kidney cells.

FIG. 17, panels A and B depict in vitro cytotoxicity of Affy7 parent CAR-T cells and seven affinity-reduced Affy7 variant CAR-T cell populations against primary human colon cells.

FIG. 18, panels A and B depict in vitro cytotoxicity of Affy6 parent CAR-T cells and five affinity-reduced Affy6 variant CAR-T cell populations against primary human colon cells.

### DETAILED DESCRIPTION OF THE INVENTION

The present disclosure is based on the unexpected discovery that chimeric antigen receptor (CAR)-T cells expressing an affinity tuned anti-c-MET scFv with reduced affinity for c-MET can effectively kill c-MET overexpressing cancer cells, while avoiding the undesirable effect of killing healthy cells and tissue.

### I. Chimeric Antigen Receptor with Reduced Affinity Anti-c-MET Binding Domain

One aspect of the present disclosure provides a chimeric antigen receptor (CAR) comprising an affinity-tuned anti-c-MET scFv with reduced target affinities to avoid targeting of healthy tissue with basal c-MET expression while maintaining sufficient avidity for targeting tumor tissues with high c-MET expression. The c-MET CARs disclosed herein comprise an artificial (non-naturally occurring) receptor having a binding specificity for the c-MET proto-oncogene, which is capable of triggering immune responses in immune cells upon binding to c-MET, particularly cells overexpressing c-MET.

The CARs disclosed herein comprise the c-MET binding domain, one or more costimulatory domains and an activation domain comprising a plurality of immunoreceptor tyrosine-based activation motifs (ITAMs), such as a CD3ζ signaling domain (also referred to as CD3 zeta). The CAR may also have a hinge domain, a transmembrane domain, or a combination thereof. In some embodiments, the transmembrane domain is located between the extracellular antigen binding domain and the costimulatory domain and the hinge domain may be located between the transmembrane domain and the costimulatory domain.

In one embodiment, provided herein is a CAR comprising an anti-c-MET scFv, one or more costimulatory domains from 4-1BB, CD28, and/or OX-40, and an ITAM-containing activation domain, such as a CD3ζ signaling domain.

### Extracellular Antigen Binding Domain

The CAR constructs disclosed herein comprise an affinity tuned extracellular antigen binding domain with reduced affinity for the c-MET proto-oncogene. The affinity tuned antigen binding domain is derived from an antibody and comprises a heavy chain variable (V_{H}) region and a light chain variable (V_{L}) region. The V_{H} and V_{L} regions herein are further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also the Human Genome Mapping Project Resources at the Medical Research Council in the United Kingdom and the antibody rules described at the Bioinformatics and Computational Biology group website at University College London. Amino acid and nucleotide sequences corresponding to the VH, VL and CDR sequences are shown in Table 18. The CDR regions in Table 18 were identified through NCBI Blast.

In some embodiments, the V_{H} and V_{L} regions are “humanized.” Humanized variable regions (or antibodies) are derived from chimeric immunoglobulins, human immunoglobulins, immunoglobulin chains, or antigen-binding fragments thereof in which minimal sequences are derived from a non-human immunoglobulin. In some embodiments, the humanized variable regions (or antibodies) comprise residues from CDRs of a non-human species (donor antibody) such as mouse, rat, or rabbit having the desired specificity, affinity, and capacity, where the framework region (FR) residues from the non-human species are replaced by corresponding human residues. Furthermore, the humanized binding region may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of human immunoglobulin consensus sequences.

In some embodiments, the EC50 of the c-MET binding domain in the c-MET CAR to c-MET is greater than 5 μM, greater than 15 μM, greater than 30 μM, greater than 60 μM, greater than 150 μM, greater than 300 μM, greater than 600 μM but less than 800 μM, or any range thereof. In other embodiments, the fold change in EC50 relative to a corresponding wild-type c-MET binding domain in the c-MET CAR (i.e. from which the reduced affinity binding domain is derived) is greater than 50-fold, greater than 150-fold, greater than 500-fold, greater than 1000-fold, greater than 2000-fold, greater than 6000-fold but less 8000-fold, or any range thereof.

In some embodiments, the extracellular binding domain (e.g., anti-c-MET scFv) comprises a V_{H} region having HC CDR1 and HC CDR2 regions comprising amino acid sequences set forth SEQ ID NOs: 1 and 2, respectively, and an HC CDR3 region comprising an amino acid sequence set forth in any one of SEQ ID NOs: 3-8; and a V_{L} region having LC CDR1, LC CDR2, and LC CDR3 regions comprising amino acid sequences set forth in SEQ ID NOs: 9-11, respectively. In certain embodiments, the HC CDR3 comprises an amino acid sequence set forth in SEQ ID NO: 5 or SEQ ID NO: 8. In some embodiments, the V_{H} and V_{L} region amino acid sequences are selected from: (a) SEQ ID NOs: 13 and 19, respectively; (b) SEQ ID NOs: 14 and 19, respectively; (c) SEQ ID NOs: 15 and 19, respectively; (d) SEQ ID NOs: 16 and 19, respectively; (e) SEQ ID NOs: 17 and 19, respectively; or (f) SEQ ID NOs: 18 and 19, respectively. In some embodiments, the extracellular binding domain comprises an anti-c-MET scFv comprising an amino acid sequence set forth in any one of SEQ ID NOs: 29-34. In some embodiments, the extracellular binding domain comprises an anti-c-MET scFv encoded by a nucleotide sequence set forth in any one of SEQ ID NOs: 36-41.

In some embodiments, the extracellular binding domain (e.g., anti-c-MET single-chain antibody) comprises a V_{H} region having HC CDR1 and HC CDR2 regions comprising amino acid sequences set forth in SEQ ID NOs: 70 and 71, respectively, and an HC CDR3 region comprising an amino acid sequence set forth in any one of SEQ ID NOs: 72-75; and a V_{L} region having LC CDR1, LC CDR2, and LC CDR3 regions comprising amino acid sequences set forth in SEQ ID NOs: 76-78, respectively. In some embodiments, the V_{H} and V_{L} region amino acid sequences are selected from: (a) SEQ ID NOs: 80 and 84, respectively; (b) SEQ ID NOs: 81 and 84, respectively; (c) SEQ ID NOs: 82 and 84, respectively; or (d) SEQ ID NOs: 83 and 84, respectively. In some embodiments, extracellular binding domain comprises an anti-c-MET scFv comprising an amino acid sequence set forth in any one of SEQ ID NOs: 92-95.

In some instances, the extracellular antigen binding domain is a single-chain antibody fragment (scFv) comprising V_{H} and V_{L} regions connected by a peptide linker. In some embodiments, the scFv has a V_{H}→V_{L} orientation. In other embodiments, the scFv has a V_{L}→V_{H} orientation. In some embodiments, the anti-c-MET scFv comprises a linker between the V_{H} and V_{L} regions. Peptide linkers used in scFv constructs are well known in the art and include, for example, the amino acid sequence (GxS)n in which x is an integer between 1-6, inclusive, and n is an integer between 1-10, inclusive. In some embodiments, the linker comprises the amino acid sequence (GGGS)n in which n is an integer between 1-10, inclusive (SEQ ID NO: 143). In one embodiment, the linker comprises the amino acid sequence multimer GGGGSGGGGSGGGGS (SEQ ID NO: 121).

In some embodiments, the affinity tuned extracellular binding domain against c-MET comprises a single domain antibody (sdAb), such as a VHH fragment, a single chain Fab fragment, a single chain Fab′ fragment, or a c-MET binding peptide.

### Other CAR Components

In addition to the affinity tuned extracellular antigen binding domain disclosed above, the CAR constructs disclosed herein further comprise one or more costimulatory domains and an activation domain comprising one or more ITAMs (as described below), such as the CD3ζ signaling domain (which contains 3), or a combination thereof.

The c-MET CARs disclosed herein comprise one or more costimulatory domains. Costimulatory domains typically enhance and/or alter the nature of the response to activation of the activation domain. Co-stimulatory domains suitable for use in the CARs of the present disclosure are typically receptor-derived polypeptides. In some embodiments, the co-stimulatory domains homodimerize. In some embodiments, the co-stimulatory domain may be the intracellular portion of a transmembrane protein (i.e., the co-stimulatory domain may be derived from the transmembrane protein). In exemplary embodiments, the costimulatory domains are from 4-1BB (CD 137), CD28, OX40, ICOS, GITR, DAP 10, DAP12, CD27, CD30, CD40, BAFFR, HVEM, ICAM-1, LFA-1 (CD11a/CD18), CD2, CDS, CD7, CD287, LIGHT, NKG2C, NKG2D, SLAMF7, NKp80, NKp30, NKp44, NKp46, CD 160, B7-H3, and a ligand that specifically binds with CD83.

The c-MET CARs disclosed herein further comprises an activation domain. As used herein, the term “activation domain” refers to an intracellular signaling domain that can trigger the production of one or more cytokines upon activation; increases antibody-dependent cellular cytotoxicity (ADCC) and cell death; and/or increased activation and/or proliferation of CD8⁺ T cells, CD4⁺ T cells, natural killer T cells, γδ T cells, and/or neutrophil proliferation. In some embodiments, the activation domain comprises at least one (e.g., 1, 2, 3, 4, 5, 6, etc.) immunoreceptor tyrosine-based activation motif (ITAM or ITAMa) with the sequence Yxx[L/I]x₍₆₋₈₎Yxx[L/I]) present in the cytoplasmic tail of an immune signaling receptor or associated subunit to induce cell signaling. The ITAM domains for use in the CARs disclosed herein can include signaling domains from several types of immune signaling receptors, including CD3, B7 family costimulatory intracellular signaling proteins such as molecules and tumor necrosis factor receptor (TNFR) superfamily receptors; signaling domains used by NK and NKT cells, such as NKp30 (B7-H6), DAP12, NKG2D, NKp44, NKp46, DAP10, and CD3 zeta; and signaling domains from ITAM-containing human immunoglobulin receptors, such as FcαRI, FcγRI, FcγRIIA, FcγRIIIA, FcγRIIC, and FcRL5. Thus, in certain embodiments, the activation domain is from CD3 zeta (SEQ ID NO: 125), Fc epsilon receptor gamma (FCER1G), Fc gamma RIIa, FcR beta (Fc epsilon lb), CD3 gamma, CD3 delta, CD3epsilon, CD5, CD22, CD79a, CD79b, CD278 (also known as “ICOS”), FcεRI, or CD66d.

The c-MET CARs disclosed herein further comprises a transmembrane domain, a hinge (or spacer) domain, or both. The transmembrane domain facilitates insertion of the CAR into the cell membranes and can be inserted between the binding domain and a costimulatory domain. In some embodiments, the transmembrane domain can be inserted between the hinge region and the co-stimulatory domain. Any transmembrane domains and/or hinge domains commonly used in CAR constructs can be used here.

In some embodiments, the c-MET CAR further comprises a hinge between the extracellular binding domain (e.g., scFv) and the transmembrane domain. In this orientation, the hinge domain provides additional distance between the antigen binding domain and the cell membrane surface on which the CAR is expressed. In some examples, the hinge domain may be from CD28, CD8, or an IgG, such as IgG1 or IgG4. In some embodiments, the hinge domain comprises an amino sequence set forth in any one of SEQ ID NOs: 126-128. In some embodiments, the transmembrane domain is obtained from a suitable cell-surface receptor, such as the transmembrane domain of a cell surface receptor of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19, and killer cell immunoglobulin-like receptor (KIR). In certain embodiments, the transmembrane membrane domain is from CD8 alpha, CD28, ICOS, or GITR, and optionally comprises an amino acid sequence set forth in SEQ ID NOs: 129-132, respectively.

In exemplary embodiments, the c-MET CAR comprises an amino acid sequence set forth in any one of SEQ ID NOs: 43-48, 50-54, 102-105, or 107-110.

In another aspect, the present disclosure provides a bispecific c-MET-CAR (or tandem c-MET-CAR (tanCAR)) comprising an additional extracellular antigen binding domain to provide improved specificity. In some embodiments, the present disclosure further contemplates a multi-specific c-MET-CAR targeting three of more antigens.

In one embodiment, the second extracellular binding domain in the bispecific CAR or tanCAR comprises an antibody fragment, such as an scFv. In some embodiments, within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁ VH₁-VH₂-VL₂. In some embodiments, a linker is disposed between the two antibodies or antibody fragments (e.g., scFvs), for example, between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₃-Ser)n or (Gly₄-Ser)n linker, wherein n is 1, 2, 3, 4, 5, or 6. In general, the linker between the two scFvs should be long enough to avoid mispairing between the domains of the two scFvs. In some embodiments, a linker is disposed between the VL and VH of the first scFv. In some embodiments, a linker is disposed between the VL and VH of the second scFv. In constructs that have multiple linkers, any two or more of the linkers may be the same or different. Accordingly, in some embodiments, a bispecific CAR or tanCAR comprises VLs, VHs, and may further comprise one or more linkers in an arrangement as described herein.

In some embodiments, immune effector cells can be genetically modified one or more CARs recognizing target cells with combinatorial Boolean logic: one can engineer T cells with multi-receptor circuits that function as AND gates (requiring two antigens to be present), OR gates (requiring the presence of one of two possible antigens), and NOT gates (high expression of one antigen, low expression of another) to increase tumor selectivity by limiting cross-reactivity with healthy tissues that also express the CAR/TCR target antigen. Exemplary target antigen combinations for AND and AND-NOT logic gates are disclosed in Table 1 of WO 2022/036133 where an “AND” precedes or follows a target antigen present on the surface of a target cancer cell and a “NOT” precedes an antigen that that is not present on the surface of a target cancer cell, but may be present on the surface of a non-cancerous cell.

Where a target antigen pair (or triple) provides for an AND logic gate, two (or three) antigens must be present on the surface of a target cancer cell in order for a genetically modified cytotoxic immune cell of the present disclosure to kill the target cancer cell, where in this case the genetically modified cytotoxic immune cell is genetically modified to express two or three antigen-triggered polypeptides, each recognizing one of the target antigens of the target antigen pair/triplet. For example, where a target antigen pair provides an AND gate logic, each of the target antigens of the target antigen pair must be present on the surface of a target cancer cell in order for a genetically modified cytotoxic immune cell of the present disclosure to kill the target cancer cell.

Where a target antigen pair/triple provides an AND-NOT logic gate (or, correspondingly, a NOT-AND logic gate), a genetically modified cytotoxic immune cell of the present disclosure: a) is activated to kill a target cancer cell that expresses the AND target cell surface antigen (e.g., the first target cell surface antigen), but not the NOT target cell surface antigen (e.g., the second and/or third target cell surface antigen), on its cell surface; and b) is inhibited from killing a non-cancerous cell if the non-cancerous cell expresses both the AND target cell surface antigen and the NOT target cell surface antigen(s) on its cell surface. In these cases, the genetically modified cytotoxic immune cell must express at least a first antigen-triggered polypeptide that specifically binds the AND target antigen of the target antigen pair and a second triggered polypeptide that specifically binds the NOT antigen of the target antigen pair. For example, in some cases, binding of an antigen-triggered polypeptide to the NOT target cell surface antigen (expressed on a non-cancerous cell) inhibits T cell activation. In this manner, unintended/undesired killing of a non-cancerous cell is reduced, because the target cancer cell expressing the AND target antigen and not the NOT target antigen will be preferentially killed over the non-cancerous cell expressing both the AND target antigen and the NOT target antigen. Since the cancer cell does not express the NOT target cell surface antigen (expressed on a non-cancerous cell), binding of the first antigen-triggered polypeptide to the AND target antigen (present on the cancer cell surface) results in activation of the genetically modified cytotoxic T cell and killing of the cancer cell.

In some embodiments, the second extracellular binding is an scFv. In other embodiments, the second extracellular binding domain is a binding domain of a protein ligand. In some embodiments, the second extracellular binding domain targets a tumor-associated antigen (TAA), particularly one that is overexpressed in solid tumors and metastatic tumors of renal, lung, thyroid, and gastrointestinal tissues. Exemplary solid tumor antigen targets for the second binding domain may include but are not limited to target antigens selected from ICAM-1, EpCAM, CEACAM5, EGFRvIII, mesothelin, CS-1, GD2, Tn Ag, PSMA, TAG72, CD44v6, CEA, KIT, IL-13Ra2, GD3, CD171, IL-lRa, PSCA, VEGFR2, Lewis Y, CD24, PDGFR-beta, SSEA-4, folate receptor alpha, ERBB2, Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumam, HPV E6 or E7, CLDN6, TSHR, GPRC5D, ALK, Polysialic acid, PLACl, globoH, NY-BR-1, UPK2, HAVCR1, ADRB3, PANX3, GPR20, Ly6k, OR51E2, TARP, and GFRa4.

In certain embodiments, the second extracellular antigen binding domain comprises an ICAM-1 binding domain. In one embodiment, the ICAM-1 binding domain comprises the I domain of the αL subunit (e.g., SEQ ID NO: 134) of human lymphocyte function-associated antigen-1 (LFA-1), which is known to target ICAM-1. A wild-type (WT) I domain encompasses amino acid residues 130-310 (SEQ ID NO: 134) of the 1145 amino acid long mature αL integrin subunit protein (SEQ ID NO: 133, which corresponds to amino acid residues 26-1170 of GenBank Accession No. NP_002200).

In some embodiments, the ICAM-1 binding domain comprises an I domain mutant disclosed in U.S. Pat. No. 10,428,136, which is incorporated herein by reference in its entirety. Such mutants differ in their affinity for ICAM-1. For example, I domain mutants having one mutation at F292A (Kd 20 μM), F292S (Kd 1.24 μM), L289G (Kd 196 nM), F265S (Kd 145 nM), and F292G (Kd 119 nM), or having two mutations at K287C/K294C (Kd 100 nM) in the wild-type I domain are suitable for the present invention. The above numbering of the amino acid residues is in reference to the amino acid sequence of the mature αL integrin of SEQ ID 133. Thus, in some embodiments, the I domain in the bispecific or multi-specific c-MET-CAR set forth in SEQ ID NO: 134 includes one mutation of F292A, F292S, L289G, F265S, or F292G, or with two mutations at K287C/K294C.

In a specific example, the CAR construct comprises, from the N-terminus to C-terminus, a c-Myc tag, an anti-c-MET scFv described herein, a CD8 hinge domain, a CD28 transmembrane domain, a CD28 co-stimulatory domain, and a CD3ζ signaling domain. In some embodiments, the CAR construct further comprises a ribosome skipping element (e.g., P2A) following the CD3ζ signaling domain and an SSTR2 coding region operably linked (i.e. fused in frame) to the ribosome skipping element. Exemplary c-MET-CARs suitable for use according to the present disclosure are set forth in SEQ ID NOs: 43-48 (encoded by SEQ ID NOs: 57-62, respectively), SEQ ID NOs: 50-55 (encoded by SEQ ID NOs: 64-69, respectively), SEQ ID NOs: 102-105 (encoded by SEQ ID NOs: 112-115, respectively), and SEQ ID NOs: 107-110 (encoded by SEQ ID NOs: 117-120, respectively). Additionally, the CAR typically comprises a signal peptide at the N-terminus of the CAR precursor protein, which is operably linked to the c-MET binding domain.

In some embodiments, the other CAR components in section (B) may be variants of their naturally occurring counterparts. In some embodiments, any of the other CAR components may contain an amino acid sequence at least 80% (e.g., at least 85%, 90%, 95%, 98%, 99% or above) identical to its natural counterpart. As used herein, the “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some examples, the costimulatory or activation domain contain up to 15 (e.g., up to 12, 10, 8, 6, 5, 4, 3, 2, or 1) amino acid residue substitutions relative to the wild-type counterpart. In some examples, the amino acid residue substitutions are conservative amino acid residue substitutions. As used herein, a “conservative amino acid substitution” refers to an amino acid substitution that does not alter the relative charge or size characteristics of the protein in which the amino acid substitution is made. Variants can be prepared according to routine methods for altering polypeptide sequences known to those skilled in the art Conservative substitutions of amino acids may include substitutions within the following groups: ((a) A→G, S; (b) R→K, H; (c) N→Q, H; (d) D→E, N; I C→S, A; (f) Q→N; (g) E4→D, Q; (h) G4→A; (i) H→N, Q; (j) I→L, V; (k) L→I, V; (l) K→R, H; (m) M→L, I, Y; (n) F→Y, M, L; (o) P→A; (p) S→T; (q) T→S; I W→Y, F; (s) Y→W, F; and (t) V→I, L.

### II. Genetically Modified Immune Cells

In another aspect, the present disclosure provides a population of immune cells comprising genetically modified immune cells (e.g., T cells) expressing one or more of the chimeric antigen receptor (CAR) constructs disclosed herein. Such modified immune cells express a CAR which specifically binds c-MET, thereby eliminating the target disease cells via, e.g., the effector activity of the immune cells. In some embodiments, the population of immune cells comprises genetically modified cytotoxic effector cells (CAR-T cells) independently expressing a single CAR, a bispecific or multispecific CAR, or multiple CARs (e.g., dual CAR-T).

In some embodiments, the immune cells for gene transduction herein may be T cells, natural killer (NK) cells, tumor infiltrating lymphocytes, dendritic cells, monocytes, macrophages, B cells, neutrophils, eosinophils, basophils, mast cells, myeloid-derived suppressor cells, mesenchymal stem cells, precursors thereof, subtypes thereof, or combinations thereof.

T cells may be selected from the group consisting of cytotoxic T-lymphocytes (CD8+), including Tc1, Tc2, Tc9, Tc17, and Tc22 T cells; helper T-lymphocytes (CD4+), including Th1, Th2, Th17, Th9, and Tfh T cells; antigen-inexperienced naïve T cells (T_{N}), stem cell memory T cells (Tscm or T_{SCM}), central memory T cells (Tcm or T_{CM}), effector memory T cells (Tem or T_{EM}), effector T cells (Teff, T_{EFF} or T_{E}), precursors to an exhausted T cell (Tpex or T_{PEX}), or exhausted T cells (Tex or T_{EX}), central memory T cells, effector memory T cells, tissue resident memory T cells, virtual memory T cells, natural killer T cells (NKT cells), FOXP3+ T cells, FOXP3− T cells). T cells may be purified from peripheral blood lymphocytes by methods known to those skilled in the art. In some embodiments, T cells may be cultured, expanded, differentiated or de-differentiated to obtain particular T cell subsets prior to or following transduction, such as antigen-inexperienced naïve T cells (T_{N}), stem cell memory T cells (Tscm or T_{SCM}), and/or central memory T cells (Tcm or T_{CM}).

In some embodiments, the immune cells are stem cells or are derived from stem cells. The stem cells can be adult stem cells, non-human embryonic stem cells, more particularly non-human stem cells, mesenchymal stem cells, cord blood stem cells, progenitor cells, bone marrow stem cells, induced pluripotent stem cells, totipotent stem cells or hematopoietic stem cells. Representative human cells are CD34+ cells. In other embodiments, the immune cell is derived from the differentiation of a population of induced pluripotent cells (iPSCs).

In some embodiments, the immune cells are harvested directly from a subject, e.g., a human subject. The cells are genetically modified as described herein and the genetically engineered immune cells are infused back into the same subject, for example, in a CAR-T cell therapy. In this case, the genetically engineered immune cells are autologous to the subject receiving the CAR-T cell therapy. In another embodiment, the immune cells are harvested directly from a donor subject, modified, and the genetically engineered immune cells are infused into a recipient subject in need of therapy, e.g., a CAR-T cell therapy. In this case, the donor immune cells are HLA-matched to the recipient subject, i.e., the cells are allogeneic to the recipient subject. In some embodiments, the immune cells are harvested and isolated from the peripheral blood of the subject (e.g., peripheral blood lymphocytes) and expanded in vitro prior to the genetic modifications disclosed herein.

In some embodiments, the CAR-expressing immune cells, including CAR-T cells, are transduced to additionally express one or more gene products, including, but not limited to ICAM-1. In some embodiments, the immune cells (e.g., T cells) are transduced to additionally express human somatostatin receptor 2 (SSTR2).

In some embodiments, the population of immune cells comprising the CAR-expressing cells further includes a second population of immune cells. In some embodiments, the second population of immune cells includes non-transduced immune cells, immune cells expressing another CAR, and/or immune cells expressing another gene product.

In some embodiments, the population of immune cells comprising the CAR disclosed herein may comprise at least about 10%, 20%, 30%, 40%, 50%, 60%, 70%, 80%, 90%, or more of the total immune cell population, or a range between two of the foregoing amounts. In one embodiment, about 50-70% of the immune cells may express the CAR.

### III. Methods of Preparing Modified Immune Cells

The c-MET CAR and optionally other exogenous nucleic acids can be introduced into suitable immune cells by routine methods and/or approaches. To generate modified immune cells expressing the c-MET-directed CARs described herein, coding sequences from one or more CARs, and optionally other gene products, may be cloned into a suitable expression vector (e.g., including but not limited to lentiviral vectors, retroviral vectors, adenoviral vectors, adeno-associated vectors, PiggyBac transposon vector and Sleeping Beauty transposon vector) and introduced into host immune cells using conventional recombinant technology known to those skilled in the art. As a result, modified immune cells of the present disclosure may comprise one or more exogenous nucleic acids encoding at least one CAR and optionally one or more other gene products described herein. In some instances, the coding sequences of such molecules are integrated into the genome of the cell for expression using viral expression vectors (e.g., lentivirus vectors) or by gene editing into suitable target sites. In other instances, the coding sequences of such molecules are not integrated into the genome of the cell.

An exogenous nucleic acid comprising a coding sequence of interest may further comprise a suitable promoter, which can be in operable linkage to the coding sequence. A promoter, as used herein, refers to a nucleotide sequence in a nucleic acid to which RNA polymerase can bind to initiate the transcription of a DNA coding region into mRNA, which will then be translated into the corresponding protein. A promoter is considered to be “operably linked” to a coding sequence when it is in a correct functional location and orientation relative to the coding sequence to control (“drive”) transcriptional initiation to produce the mRNA for translating the protein. In some instances, the promoter described herein can be constitutive, which initiates transcription independent other regulatory factors. In some instances, the promoter described herein can be inducible, which is dependent on regulatory factors for transcription.

Additionally, the exogenous nucleic acids described herein may further contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA.

In some instances, one or more nucleic acids encoding the CAR(s) and/or other gene products disclosed herein can be inserted into a suitable expression cassette in a multi-cistronic manner such that the various molecules are expressed as separate polypeptides. In some examples, an internal ribosome entry site (IRES) can be inserted between two coding sequences to achieve this goal. Alternatively, a nucleotide sequence coding for a self-cleaving peptide (e.g., T2A or P2A) can be inserted between two coding sequences as described above.

For example, in some embodiments, T cells may be transduced with a nucleic acid encoding a second gene product, such as SSTR2, and a CAR disclosed herein. In certain embodiments, the nucleic acid encodes an SSTR2 polypeptide (aa 1-381; SEQ ID NO: 137) or a truncated SSTR2 polypeptide (aa 1-314; SEQ ID NO: 138). In a specific embodiment, the nucleic acid encodes a SSTR2-CAR fusion protein comprising an amino acid cleavage sequence between the CAR coding sequence and the SSTR2 coding sequence. The cleavage sequence may encode a self-cleaving 2A peptide from porcine teschovirus-1 (P2A; SEQ ID NO: 139), equine rhinitis A virus (E2A; SEQ ID NO: 140), Thosea asigna virus (T2A; SEQ ID NO: 141), or foot-and-mouth disease virus (F2A; SEQ ID NO: 142), or a combination thereof.

SSTR2 expressing cell compositions and methods for using SSTR2 as a reporter for CAR-T cell monitoring and use in cancer are disclosed in U.S. Pat. No. 10,577,408, which is expressly incorporated by reference herein.

### IV. Therapeutic Applications

### (A) Adoptive CAR-T Cell Therapy

In one aspect, immune cell populations comprising the modified immune cells as described herein may be used in an adoptive immune cell therapy (e.g., CAR-T) for treating a target disease, such as a solid tumor or tumor characterized by overexpression of c-MET. In an embodiment, a method for treating cancer comprises administering to a subject in need thereof, a population of immune cells comprising genetically engineered CAR-expressing cells (e.g., T cells) described herein in an amount suitable for treating the cancer.

To practice the therapeutic methods described herein, an effective amount of the immune cell population comprising the genetically modified immune cells described herein may be administered to a subject in need of cancer treatment via a suitable route of administration (e.g., intravenous infusion). One or more of the immune cell populations may be mixed with a pharmaceutically acceptable carrier to form a pharmaceutical composition prior to administration, which is also within the scope of the present disclosure. The immune cells may be autologous to the subject, e.g., obtained from the subject in need of the treatment, modified to express the c-MET CAR construct and optionally one or more additional exogenous gene products. The resultant modified immune cells can then be administered to the same subject. Administration of autologous cells to a subject may result in reduced rejection of the immune cells as compared to administration of non-autologous cells. Alternatively, the immune cells can be allogeneic cells, e.g., the cells are obtained from a first subject, modified as described herein and administered to a second subject that is different from the first subject but of the same species. For example, allogeneic immune cells may be derived from a human donor and administered to a human recipient who is different from the donor.

The subject to be treated may be a mammal (e.g., human, mouse, pig, cow, rat, dog, guinea pig, rabbit, hamster, cat, goat, sheep, or monkey) suffering from cancer, particularly a human patient with a cancer characterized by overexpression of c-MET.

The term “an effective amount” as used herein refers to the amount of each active agent required to confer therapeutic effect on the subject, either alone or in combination with one or more active agents. Effective amounts may vary, as recognized by those skilled in the art, depending on the particular condition being treated, the severity of the condition, toxicity consideration, previous trial results, individual patient parameters including age, physical condition, size, gender and weight, the duration of treatment, route of administration, excipient usage, co-usage (if any) with other active agents and like factors within the knowledge and expertise of the health practitioner. The quantity to be administered depends on the subject to be treated, including, for example, the capacity of the individual's immune system to produce a cell-mediated immune response. Effective amounts of the genetically engineered CAR-T cells required to be administered depend on the judgment of the practitioner. However, suitable dosage ranges are readily determinable by one skilled in the art.

The term “treating” as used herein refers to the application or administration of a cell composition to a subject with cancer with the purpose to cure, heal, alleviate, relieve, alter, remedy, ameliorate, improve, affect progression of the cancer and its symptoms.

An effective amount of the immune cells may be administered to a human patient in need of the treatment via a suitable route, e.g., intravenous infusion. In some instances, about 1×10⁶ to about 1×10⁸ CAR+ T cells may be given to a human patient (e.g., a leukemia patient, a lymphoma patient, or a multiple myeloma patient). In some examples, a human patient may receive multiple doses of the immune cells. For example, the patient may receive two doses of the immune cells on two consecutive days. In some instances, the first dose is the same as the second dose. In other instances, the first dose is lower than the second dose, or vice versa.

In any of the treatment methods involving the use of the modified immune cells disclosed herein, the subject may be administered IL-2 concurrently with the cell therapy. More specifically, an effective amount of IL-2 may be given to the subject via a suitable route before, during, or after the cell therapy. In some embodiments, IL-2 is given to the subject after administration of the immune cells. In some embodiments, prior to the cell therapy, the subject receives a lymphodepleting treatment to condition the subject for the cell therapy.

In some embodiments, the cancer for treatment is a solid tumor. In some embodiments, the solid tumor is a carcinoma. In certain embodiments, the carcinoma is an adenocarcinoma or squamous cell carcinoma thereof. Examples of carcinomas include small cell lung carcinoma, non-small cell lung carcinoma, squamous cell lung carcinoma, large cell lung carcinoma, pancreatic carcinoma, pancreatic ductal carcinoma, prostate carcinoma, esophageal carcinoma, breast carcinoma, ovarian carcinoma, prostate carcinoma, colorectal carcinoma, bladder carcinoma, cervical carcinoma, hepatocellular carcinoma, renal hepatocellular carcinoma, gastric carcinoma, papillary carcinoma, adrenocortical carcinoma, pituitary carcinoma, a head and neck carcinoma, an adenocarcinomas thereof, a squamous cell carcinomas thereof, and a metastatic cancer thereof. In some embodiments, the solid cancer is a glioblastoma or mesothelioma.

In some embodiments, the solid tumor expresses one or more antigen selected from the group consisting of: EpCAM, CEACAM5, EGFRvIII, mesothelin, CS-1, GD2, Tn Ag, PSMA, TAG72, CD44v6, CEA, KIT, IL-13Ra2, GD3, CD171, IL-1Ra, PSCA, VEGFR2, Lewis Y, CD24, PDGFR-beta, SSEA-4, folate receptor alpha, ERBB2, Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumam, HPV E6 or E7, CLDN6, TSHR, GPRC5D, ALK, Polysialic acid, PLAC1, globoH, NY-BR-1, UPK2, HAVCR1, ADRB3, PANX3, GPR20, Ly6k, OR51E2, TARP, and GFRa4. In some embodiments, these antigens may serve as target antigens for the bispecific c-MET CARs or as secondary CARs to be used in conjunction with the c-MET CARs disclosed herein.

### (B) Co-Administration of Tyrosine Kinase Inhibitors.

It is known that transient inhibition or modulation of TCR signaling and/or CAR signaling in human T cells can prevent or reverse T cell exhaustion and restore T cell function to CAR-T cells undergoing functional exhaustion. In particular, in vivo treatment of the CAR-T cells described herein with certain tyrosine kinase inhibitors inhibiting T cell receptor signaling (e.g., a Lck tyrosine kinase inhibitor (e.g., dasatinib)) can suppress exhaustion marker expression, augment memory formation, decrease expression of PD1 on CAR-T cells, and facilitate cell survival/proliferation as described in US 2020/101108 A1 and US 2021169880 A1, the disclosures of which are expressly incorporated by reference herein. Similar findings have been obtained using small molecules having a thiazole, imidazolepyridiazine or piperazinyl-methyl-aniline structure (US 2021/0393628 A1, incorporated by reference herein).

Thus, in some embodiments, a patient receiving CAR-T treatment in accordance with the present disclosure may be additionally administered a tyrosine kinase or small molecule having a thiazole, imidazolepyridiazine or piperazinyl-methyl-aniline structure to mitigate T cell exhaustion, augment memory T cell formation, and/or maintain and facilitate cell survival and proliferation. In other embodiments, these active agents may be used to improve ex vivo cell expansion and collection of CAR-T populations that are resistant and/or less prone to T cell exhaustion. Thus, in another aspect, the present disclosure further provides cell compositions comprising a population of CAR-T cells that are expanded in the presence of particular compounds described herein.

Exemplary tyrosine kinases for administration or cell treatment include dasatinib, ponatinib, saracatinib, bosutinib, nilotinib, and combinations thereof. Exemplary small molecules having a thiazole, imidazolepyridiazine or piperazinyl-methyl-aniline structure are disclosed in US 2021/0393628 A1, the disclosures of which are expressly incorporated by reference herein.

Such methods are not limited to particular manner of administration. In some embodiments, multiple cycles of treatment are administered to the subject. In some embodiments, the pharmaceutical composition is administered intermittently. In some embodiments, the pharmaceutical composition is administered for a period of time sufficient to restore at least partial T cell function then discontinued. In some embodiments, the pharmaceutical composition is administered orally.

In some embodiments, the pharmaceutical compositions are administered iteratively for purposes of facilitating periods of CAR-T cell inactivation (e.g., during pharmaceutical composition administration) and periods of CAR-T cell activation (e.g., during absence of pharmaceutical composition administration; following clearance of the pharmaceutical composition).

In some embodiments, patients undergoing CAR-T treatment are subjected to intermittent exposure to dasatinib (or other active agents above) to reduce exhaustion, and augment the engraftment, proliferation, and persistence of CAR-T cells in vivo., as well as antitumor function of the CAR-T cells.

The terms “intermittent administration” or “administered intermittently” in connection with the tyrosine kinase inhibitors described herein refer to the use of these tyrosine kinase inhibitors in an administration regime that causes intermittent changes between a state wherein the patient has tyrosine kinase inhibitor serum levels within the therapeutic window and a state wherein the patient has tyrosine kinase inhibitor serum levels below the therapeutic window. A therapeutic window of a given tyrosine kinase inhibitor can be determined by any methods known in the art.

Alternatively, the terms “intermittent administration” and “administered intermittently” in connection with a tyrosine kinase inhibitor as used herein refer to the use of a tyrosine kinase inhibitor in an administration regime causing: (1) intermittent changes between a state where the patient has tyrosine kinase inhibitor serum levels causing complete inhibition of the tyrosine kinase and a state where the patient has tyrosine kinase inhibitor serum levels causing partial inhibition of the tyrosine kinase; (2) intermittent changes between a state where the patient has tyrosine kinase inhibitor serum levels causing complete inhibition of the tyrosine kinase and a state where the patient has tyrosine kinase inhibitor serum levels causing no inhibition of the tyrosine kinase; or (3) intermittent changes between a state where the patient has tyrosine kinase inhibitor serum levels causing partial inhibition of the tyrosine kinase and a state where the patient has tyrosine kinase inhibitor serum levels causing no inhibition of the tyrosine kinase.

Such inhibition can be measured by any methods known in the art, e.g., by measuring the activity of the tyrosine kinase itself using appropriate enzyme assays, or by measuring cellular functions downstream of the kinase. In some embodiments, a partial inhibition refers to an inhibition of at least 25% to 75% compared to a situation in the absence of the inhibitor. As used herein, “no inhibition” refers to an inhibition of less than 25%, or less than 10% compared to a situation in the absence of the inhibitor.

In the case of CAR-T cells, inhibition of less than 25% or 10% can be an inhibition of the cytotoxic lysis, cytokine secretion, and/or proliferation of the T cells. Further, the inhibition of at least 25%, but no more than 75%, can preferably be an inhibition of the cytotoxic lysis, cytokine secretion, and proliferation of the CAR-T cells.

In some embodiments, intermittent administration of dasatinib may cause intermittent changes between a state wherein the serum levels of dasatinib are above 50 nM and a state wherein the serum levels of dasatinib are at or below 50 nM. Intermittent administration may be achieved by using an administration interval longer than the terminal phase half-life of the tyrosine kinase inhibitor, longer than 2 times the terminal phase half-life of the tyrosine kinase inhibitor, or longer than 3 times, 4 times, or 5 times the terminal phase half-life of the tyrosine kinase inhibitor. For example, intermittent administration of dasatinib may be achieved using an administration interval of at least 6 hours for dasatinib or at least 12 hours for dasatinib. It will be understood by a person skilled in the art that for each administration regime, appropriate dosages of the respective tyrosine kinase inhibitors can be selected based on pharmacokinetic and pharmacodynamic experiments.

The terms “continuous administration” or “administered continuously” in connection with a tyrosine kinase inhibitor as used herein refer to the use of said tyrosine kinase inhibitor in an administration regime that causes a complete inhibition of the tyrosine kinase in a continuous manner. According to the invention, a complete inhibition refers to an inhibition of at least 75%, compared to a situation in the absence of the inhibitor. Such inhibition can be measured by any methods known in the art, e.g., by measuring the activity of the tyrosine kinase itself using appropriate enzyme assays, or by measuring cellular functions downstream of said kinase.

In the case of CAR-T cells, inhibition of at least 75% can be an inhibition of the cytotoxic lysis, cytokine secretion, and proliferation of T cells. Alternatively, the terms “continuous administration” and “administered continuously” in connection with a tyrosine kinase inhibitor described herein refer to use of the tyrosine kinase inhibitor in an administration regime that results in serum levels of the tyrosine kinase which are continuously within the therapeutic window. In some embodiments, continuous administration of dasatinib encompasses any administration wherein the serum levels of dasatinib are constantly maintained at or above 50 nM. In one embodiment, dasatinib is administered continuously, where the administration comprises oral administration of 50-200 mg dasatinib every 6-8 hours or 140 mg every 6 hours.

In some embodiments, the threshold serum level is within the range of 0.1 nM-1 μM, 1 nM-500 nM, 5 nM-100 nM, 10 nM-75 nM, or 25 nM-50 nM.

### (C) Monitoring CAR-T Cell Distribution in a Patient

In another aspect, the present disclosure provides a method for treating cancer and monitoring CAR-T cell distribution in a patient undergoing CAR-T cell therapy. The method involves the use of CAR-T cells co-expressing human somatostatin receptor 2 (SSTR2) as a cell surface marker for monitoring CAR-T cell distribution in a patient.

SSTR2 can be used in conjunction with FDA-approved positron emission tomography (PET) radiotracers currently used in clinics to probe for overexpressed SSTR2 in neuroendocrine tumors, specifically ⁶⁸Gallium conjugates of DOTATOC and DOTATATE. Single-photon emission computed tomography (SPECT)-based imaging is also available using ¹¹¹In-DTPAOC (Octreoscan) or ¹⁷⁷Lutetium. SSTR2 displays restricted basal expression in tissues and all major organs except in the kidneys and cerebrum making it ideal for detection of adoptively transferred CAR-T cells targeting a multitude of solid tumors.

It has previously been shown that SSTR2 facilitates rapid radiotracer uptake and this combined with swift renal clearance of unbound DOTATOC means that high quality, clinical-grade images can be obtained at one hour post DOTATOC injection. DOTATOC also has a short half-life of 68 min which, combined with its rapid clearance, delivers a low radiation dose to the patient. The fact that SSTR2 is of human origin also limits its immunogenicity which has plagued experiments using non-human genetic reporters.

SSTR2 compositions and methods for using SSTR2 as a reporter for CAR-T cell monitoring and use in cancer are disclosed in U.S. Pat. No. 10,577,408, which is expressly incorporated by reference herein.

In one embodiment, the method comprises: (a) incubating a population of CAR-T cells described herein with a radioactive label that binds to SSTR2; (b) intravenously infusing the labeled CAR-T cells into a patient in an amount of 10⁴-10⁸ cells/kg patient, and (c) detecting the labeled CAR-T cell distribution by positron emission tomography/computed tomography (PET/CT) imaging, wherein the labeled CAR-T cells are infiltrated into cancer cells to kill the cancer cells. In this method, SSTR2 is pre-labeled in vitro. In some embodiments, the labeled CAR-T cells are administered in an amount of 10⁶-10⁸ or 10⁶-10⁷ cells/kg of the patient.

In another embodiment, the method comprises: (a) intravenously infusing a population of CAR-T cells described herein into a patient; (b) injecting into the patient a radioactive label that binds to SSTR2 at least one hour prior to PET/CT imaging, and (c) detecting the labeled CAR-T cell distribution by PET/CT imaging, wherein the labeled CAR-T cells are infiltrated into cancer cells to kill the cancer cells. In this method, SSTR2 is labeled post-infusion in vivo. In certain embodiments, the CAR-T cells have been transduced to express at least 100,000 molecules of SSTR2 per T cell.

In some embodiments, the label is radioactively labeled DOTATOC or radioactively labeled DOTATATE, such as ⁶⁸Gallium-DOTATOC or ⁶⁸Gallium-DOTATATE. The methods for monitoring distribution of radiolabeled CAR-T cells may be used in connection with treating any of the cancers described herein, including but not limited to thyroid cancer, gastric cancer, pancreatic cancer, or breast cancer.

### (D) Combination Therapies

The immune cell populations comprising e.g., the CAR-T cells described herein may be utilized in conjunction with other types of therapy or active agents for cancer, such as chemotherapy, surgery, radiation, gene therapy, and so forth. Such therapies can be administered simultaneously or sequentially (in any order) with the immunotherapy described herein. When co-administered with an additional therapeutic agent, suitable therapeutically effective dosages for each agent may be lowered due to additive or synergistic effects.

In some examples, the subject is treated with an anti-cancer therapy (e.g., those disclosed herein) to reduce tumor burden prior to the CAR-T therapy disclosed herein. For example, the subject (e.g., a human cancer patient) may be treated with chemotherapy (e.g., comprising a single chemotherapeutic agent or a combination of two or more chemotherapeutic agents) at a dose that substantially reduces tumor burden. In some instances, the chemotherapy may reduce the total white blood cell count in the subject to lower than 10⁸/L, e.g., lower than 10⁷/L. Tumor burden of a patient after the initial anti-cancer therapy, and/or after the CAR-T cell therapy disclosed herein may be monitored via routine methods. If a patient showed a high growth rate of cancer cells after the initial anti-cancer therapy and/or after the CAR-T therapy, the patient may be subjected to a new round of chemotherapy to reduce tumor burden followed by any of the CAR-T therapies disclosed herein.

Non-limiting examples of other anti-cancer therapeutic agents for use in combination with the modified immune cells (e.g., CAR-T cells) described herein include, but are not limited to, immune checkpoint inhibitors (e.g., PDL1, PD1, and CTLA4 inhibitors), anti-angiogenic agents (e.g., TNP-470, platelet factor 4, thrombospondin-1, tissue inhibitors of metalloproteases, prolactin, angiostatin, endostatin, bFGF soluble receptor, transforming growth factor beta, interferon alpha, interferon gamma, soluble KDR and FLT-1 receptors, and placental proliferin-related protein); VEGF antagonists (e.g., anti-VEGF antibodies, VEGF variants, soluble VEGF receptor fragments); chemotherapeutic compounds. In some embodiments, the anti-cancer therapeutic agents include pembrolizumab (Keytruda™) ipilimumab (Yervoy™), nivolumab (Opdivo™), or atezolizumab (Tecentriq™)

Exemplary chemotherapeutic compounds include pyrimidine analogs (e.g., 5-fluorouracil, floxuridine, capecitabine, gemcitabine and cytarabine); purine analogs (e.g., fludarabine); folate antagonists (e.g., mercaptopurine and thioguanine); antiproliferative or antimitotic agents, for example, vinca alkaloids; microtubule disruptors such as taxane (e.g., paclitaxel, docetaxel), vincristin, vinblastin, nocodazole, epothilones and navelbine, and epidipodophyllotoxins; and DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide).

In some embodiments, radiation or radiation and chemotherapy is used in combination with the cell populations comprising modified immune cells described herein. Additional useful agents and therapies can be found in Physician's Desk Reference, 59th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J.

### V. General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane, Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986); B. Perbal, A practical Guide To Molecular Cloning (1984); F. M. Ausubel et al. (eds.); Chimeric Antigen Receptor (CAR) Immunotherapy (D. W. Lee and N. N. Shah, eds., Elservier, 2019, ISBN:9780323661812); Basics of Chimeric Antigen Receptor (CAR) Immunotherapy (M. Y. Balkhi, Academic Press, Elsevier Science, 2019, ISBN:9780128197479); Chimeric Antigen Receptor T Cells Development and Production (V. Picanço-Castro, K. C. R. Malmegrim, K. Swiech, eds., Springer US, 2020, ISBN:9781071601488); Cell and Gene Therapies (C. Bollard, S. A. Abutalib, M.-A. Perales eds., Springer International, 2018; ISBN:9783319543680) and Developing Costimulatory Molecules for Immunotherapy of Diseases (M. A. Mir, Elsevier Science, 2015, ISBN:9780128026755).

The present disclosure is not limited in its application to the details of construction and the arrangements of component set forth in the description herein or illustrated in the drawings. The present disclosure is capable of other embodiments and of being practice or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. As also used in this specification and the appended claims, the singular forms “a,” “an,” and “the” include plural references unless the context clearly dictates otherwise.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Generation of Reduced Affinity c-MET CAR-T Cells

Site directed mutagenesis was used to create affinity tuned anti-c-MET variant single-chain variable fragments (scFvs) comprising variable heavy (V_{H}) regions with lower affinities than scFvs comprising parental versions of the V_{H} and variable light (V_{L}) chain regions. Edits to the CDR3 of the heavy chain were made using an NEB Q5 site-directed mutagenesis kit (NEB #E0554S) with individually designed primers outlined in Table 1 to create desired amino acid changes.

TABLE 1
Primers used for making antibodies

| | | | | | Primer | | VH |
|---|---|---|---|---|---|---|---|
| | | | | | SEQ ID | | SEQ ID |
| Variant | Change | | Primer # | Primer* | NO | Tm | NOS |
| Affy-Y6A | Y → A | TAC → gcg | AB_001 | CTGTGCCACCgcgGGCTCCTACGTGAGC | 133 | 66 | 13, 19 |
| | | | AB_002 | TAGTACACGGCGCTGTOC | 134 | | |
| Affy7-L9A | L → A | TTG → gcG | AB_015 | CGTGAGCCCTgcgGATTATTGGGGC | 135 | 67 | 14, 19 |
| | | | AB_016 | TAGGAGCCGTAGGTGGCA | 136 | | |
| Affy7-Y6V | Y → V | TAC → gtC | AB_037 | CTGTGCCACCgtCGGCTCCTACG | 137 | 66 | 15, 19 |
| | | | AB_038 | TAGTACACGGCGCTGTCC | 138 | | |
| Affy7-Y6L | Y → L | TAC → ctC | AB_039 | CTGTGCCACCctCGGCTCCTAC | 139 | 65 | 16, 19 |
| | | | AB_038 | TAGTACACGGCGCTGTOC | 138 | | |
| Affy7-Y6I | Y → I | TAC → atC | AB_040 | CTGTGCCACCatCGGCTCCTAC | 140 | 65 | 17, 19 |
| | | | AB_038 | TAGTACACGGCGCTGTCC | 138 | | |
| Affy7-Y6M | Y → M | TAC → atg | AB_042 | CTGTGCCACCatgGGCTCCTACGTGAGCCC | 141 | 65 | 18, 19 |
| | | | AB_038 | TAGTACACGGCGCTGTCC | 138 | | |
| Affy6-I3A | I → A | TAC → gcC | AB_086 | CCGCTCGGAGgcCACCACCGAG | 142 | 68 | 80, 84 |
| | | | AB_087 | GCGCAGTAGTACACGGCC | 143 | | |
| Affy6-I3G | I → G | ATC → ggC | AB_120 | CCGCTCGGAGggcACCACCGAGTTC | 147 | 70 | 83, 84 |
| | | | AB_117 | GCGCAGTÅGTACACGGCC | 145 | | |
| Affy6-I3F | I → F | ATC → ttC | AB_123 | CCGCTCGGAGttcACCACCGAGT | 148 | 71 | 82, 84 |
| | | | AB_117 | GCGCAGTAGTACACGGCC | 146 | | |
| Affy6-I3W | I → W | ATC → tgg | AB_125 | CCGCTCGGAGtggACCACCGAGTTC | 149 | 67 | 81, 84 |
| | | | AB_117 | GCGCAGTAGTACACGGCC | 146 | | |

*mutant nucleotides are bolded in small case

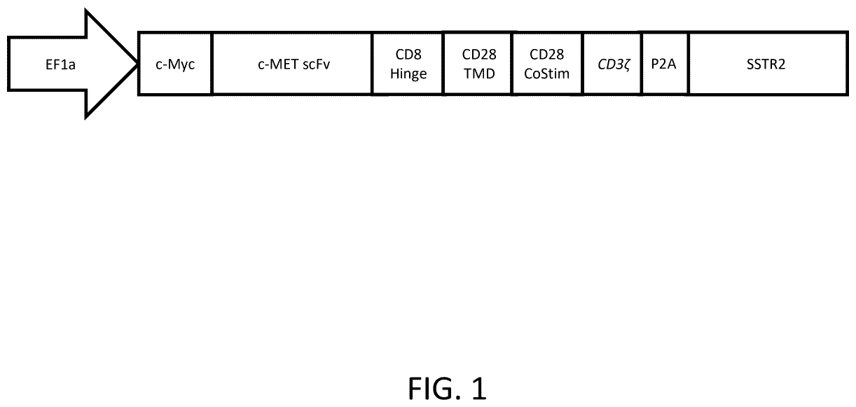

Lentivirus constructs were prepared to facilitate characterization of the binding properties of the affinity tuned anti-c-MET scFvs in chimeric artificial receptors (CARs). C-MET CARs were prepared by cloning genetic sequences encoding parental (wild-type) c-MET Affy7 and Affy6 scFvs and the affinity-tuned c-MET Affy7 and Affy6 variant scFvs into a lentiviral vector between a c-myc tag and CAR element CD8 hinge. As shown in FIG. 1, the lentiviral vector construct comprises (5′ to 3′) the EF1a promoter, c-myc tag, c-MET scFv, CD8 hinge, CD28 transmembrane domain, CD28 co-stimulatory domain, CD3 zeta signaling domain, porcine teschovirus-1 2A (P2A) ribosome skipping element, and a human somatostatin receptor 2 (SSTR2) reporter gene for CAR-T cell imaging.

To obtain stably transformed CAR-expressing cells, lentivirus particles were produced by transiently transfecting HEK 293T cells using the TransIT®-Lenti transfection reagent, (Mirus Bio, MIR6604). Briefly, 8 μg of transfer gene and 12 μg of LV-MAX lentiviral packaging mix (ThermoFisher, A43237) were mixed with Opti-MEM and 50 μL of Trans-IT and incubated for 20 minutes at room temperature. The resulting solutions were added dropwise to 10 cm² cell culture dishes seeded with 6×10⁶ HEK 293T cells in 10 mL DMEM 24 hrs. previously. Transfection media was replaced 16-24 hrs. later. Media containing lentivirus was harvested at 48 hrs. post transfection, filtered through 0.45 μm filers and concentrated by adding Lenti-X concentrator (Takara, 631231) at ⅓ of final volume of supernatant. The solution was then incubated a 4° C. for at least 3 hrs. (as long as overnight) and then spun at 1,500 g for 1 hr at 4° C. The supernatant was removed, and the pellet was resuspended in 400 μL of sterile PBS and stored at −80° C.

### Example 2: Evaluation of Binding Properties of Affinity-Tuned c-MET CARs

Binding of labeled c-MET to CAR-T cells expressing the affinity tuned c-MET scFvs was evaluated by transducing lentivirus anti-c-MET CAR particles into human T cells to form c-MET CAR-T cells incubated with AF647-labeled c-MET. Briefly, human T cells were transduced 24 hrs. post activation with ImmunoCult™ CD3/CD28 T cell activator (StemCell, 10971) in 10 mL of media containing IL7 (12.5 ng/mL), IL15 (12.5 ng/mL) and 10 μL of Lenti-BOOST (Mayflower Bioscience, SBPLV10103) in G-Rex® 6M wells (Scale Ready, 80660M). Following a 48 hr. incubation at 37° C., the culture media was brought up to 100 mL per well and incubated at 37° C. for 10 days.

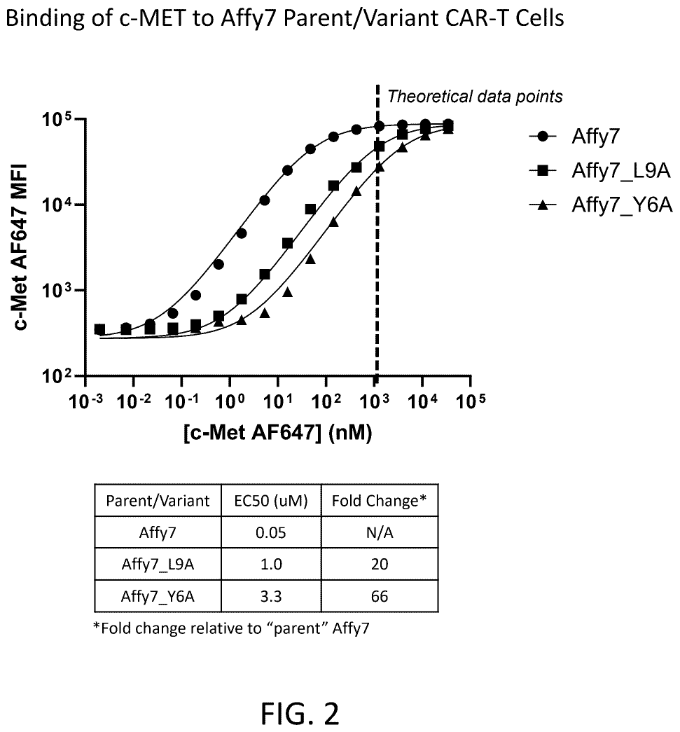

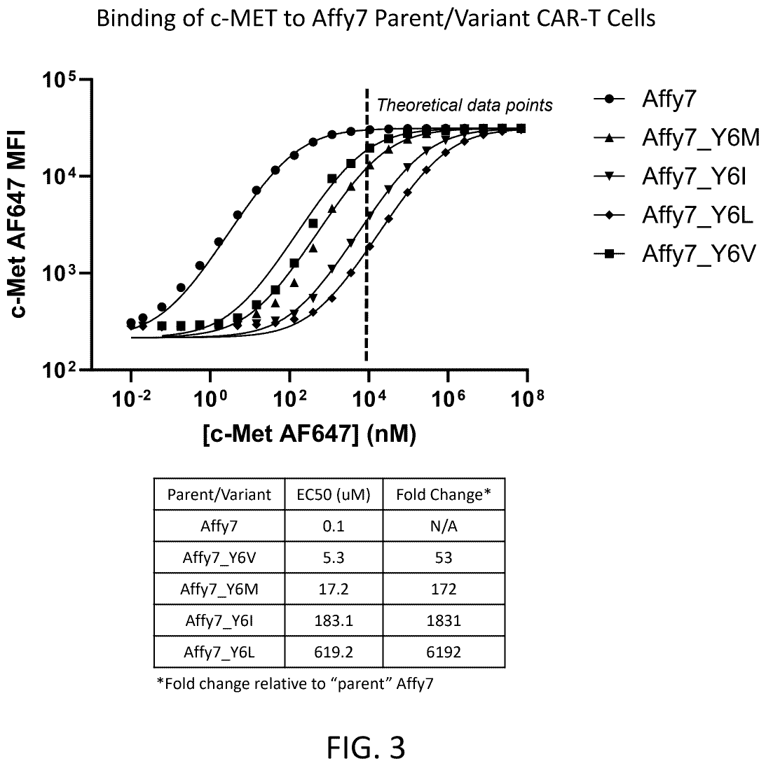

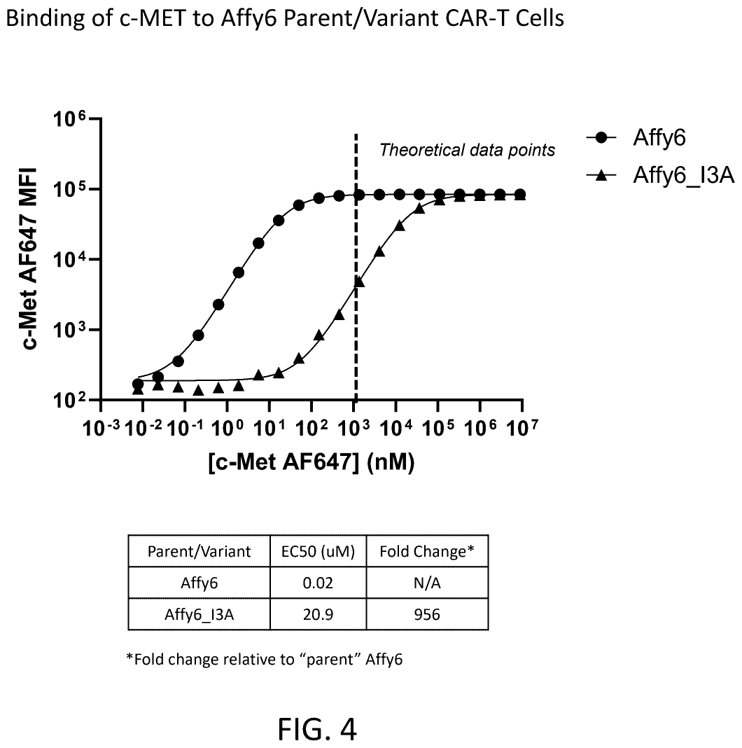

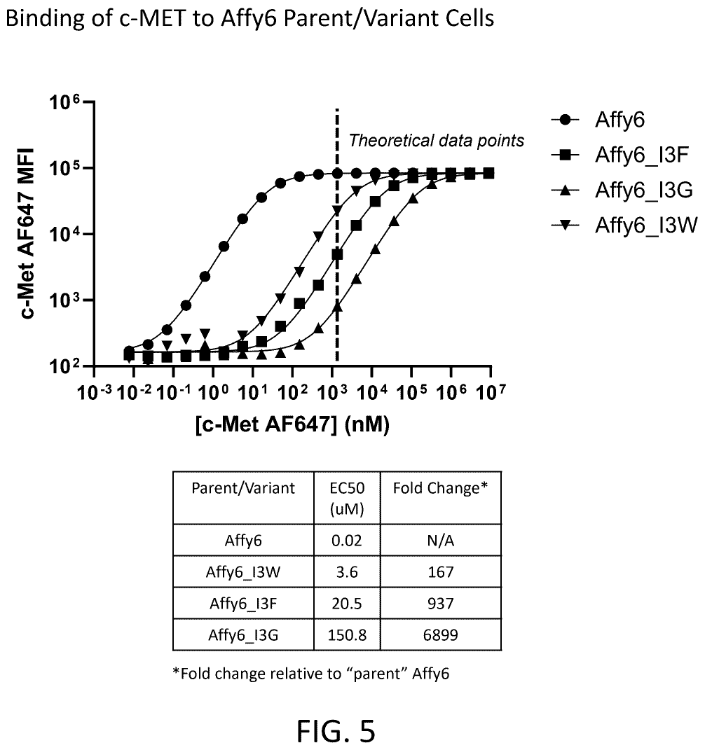

Binding curves were generated by serial diluting AF647 labeled c-met protein (AlexaFluor™ 647 antibody labeling kit, ThermoFisher, A20186) and staining each sample with c-myc antibody (Miltenyi, 130-116-485) at a 1:800 dilution. Cells were stained on ice for 30 mins and then washed with PBS. After washing, residually bound protein on CAR T cells were measured on a flow cytometer (Beckman, CytoFLEX®). The resulting binding data was fit using a non-linear regression analysis on GraphPad Prism to calculate half effective dose concentration (EC₅₀) values. The results of this analysis are shown in FIGS. 2-5 and Tables 2-5 below.

As compared to CAR-T cells expressing the parental anti-c-MET scFv binding domains (i.e., Affy7 and Affy6), CAR-T cells expressing the affinity-tuned anti-c-MET scFvs exhibiting reduced affinities for c-MET ranging between 20 to 7636-fold relative to the parental-expressing CARs (see FIGS. 2-5 and Tables 2-5).

TABLE 2
Binding Affinities of Parental- and Variant Affy7-
Based MET CAR-T Cells for c-MET (Round 1)

| Parent/Variant | EC50 (μM) | Fold Change* |
|---|---|---|
| Affy7 | 0.05 | N/A |
| Affy7-L9A | 1.0 | 20 |
| Affy7-Y6A | 3.3 | 66 |

TABLE 3
Binding Affinities of Parental- and Variant Affy7-
Based MET CAR-T Cells for c-MET (Round 2)

| Parent/Variant | EC50 (μM) | Fold Change* |
|---|---|---|
| Affy7 | 0.1 | N/A |
| Affy7-Y6V | 5.3 | 53 |
| Affy7-Y6M | 17.2 | 172 |
| Affy7-Y6I | 183.1 | 1831 |
| Affy7-Y6L | 619.2 | 6192 |

TABLE 4
Binding Affinities of Parental- and Variant Affy6-
Based MET CAR-T Cells for c-MET (Round 1)

| Parent/Variant | EC50 (μM) | Fold Change* |
|---|---|---|
| Affy6 | 0.02 | N/A |
| Affy6-I3A | 20.9 | 956 |

TABLE 5
Binding Affinities of Parental- and Variant Affy6-
Based MET CAR-T Cells for c-MET (Round 2)

| Parent/Variant | EC50 (μM) | Fold Change* |
|---|---|---|
| Affy6 | 0.02 | N/A |
| Affy6-I3W | 3.6 | 167 |
| Affy6-I3F | 20.5 | 937 |
| Affy6-I3G | 150.8 | 6899 |

### Example 3: Cytotoxicity of Reduced Affinity c-MET CAR-T Cells in Cancer Cell Lines

One of the pitfalls of CAR-T cell therapies concerns toxicities and T cell exhaustion associated with recognition of antigen both on normal, non-target cells as well as on cancer cells. However, when using reduced affinity c-MET CAR-T cells to reduce cell toxicities and T cell exhaustion, it is important to maintain effective killing of the targeted cancer cells.

To evaluate cytotoxicity of the reduced affinity c-MET CAR-T cells against human carcinoma cells, 5×10⁴ target cells (SNU638, Hs746T, and H1993) stably transduced to express GFP and firefly luciferase were co-cultured with c-met CAR-T cells at an effector to target ratio of 5:1. Co-cultures were carried out in T cell culture medium containing 150 μg/mL D-Luciferin (Fisher Scientific, NC1276267) with no cytokine supplementation. Luminescence was measured using a plate reader (BioTek®, Synergy Neo2) with readings every 24 hrs. for 3 days. In each case, readings were normalized to the no T cell control group and percent cytotoxicity was reported.

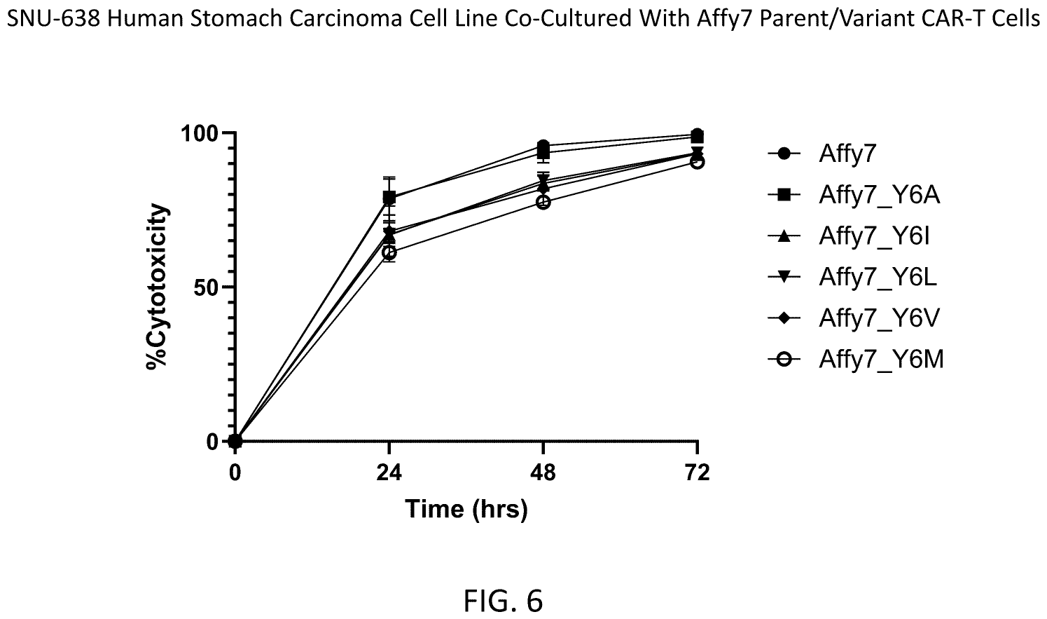

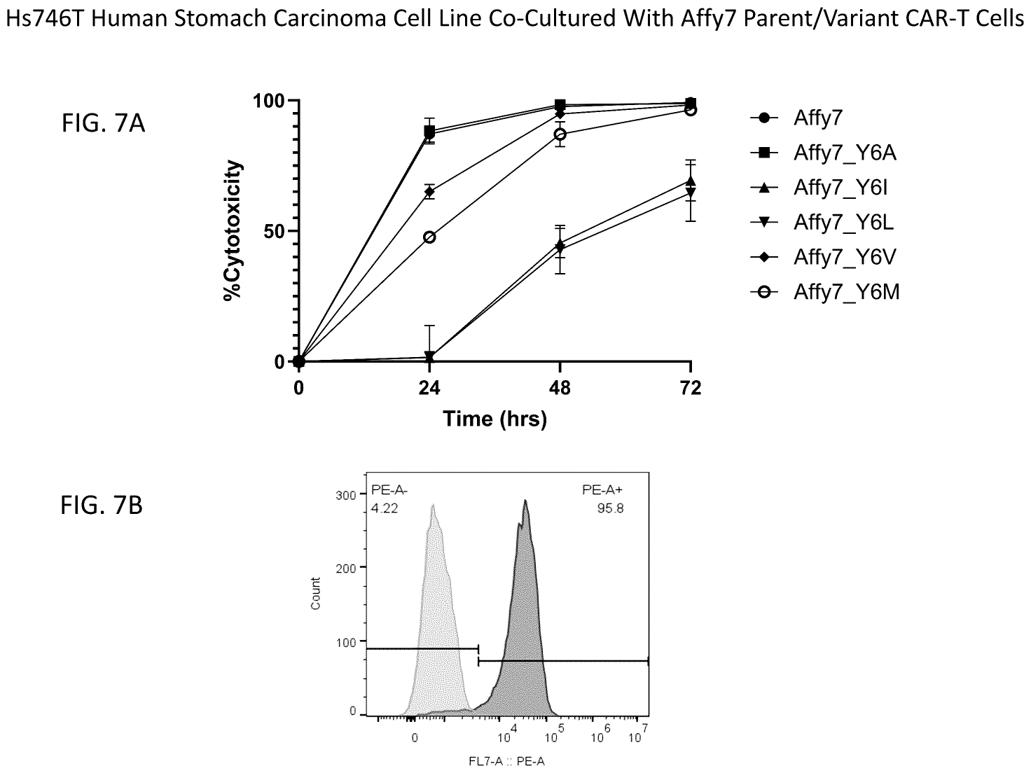

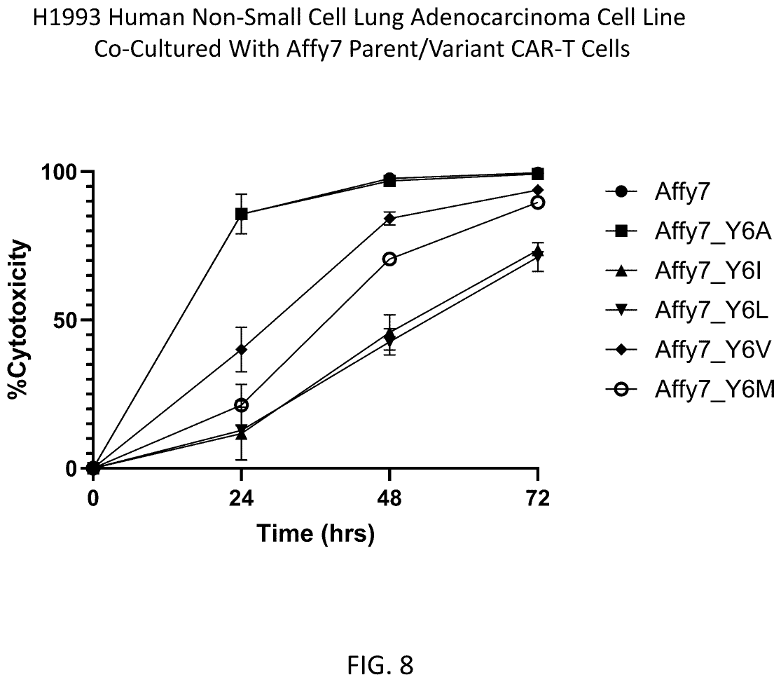

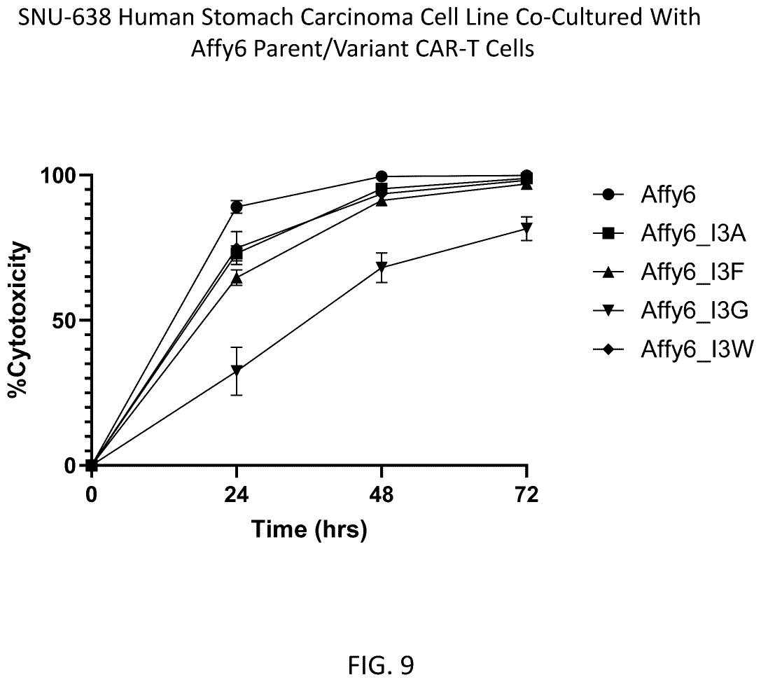

As shown in FIGS. 6-9 and Tables 6-9 below, all of the c-MET CAR-T cells of the present disclosure exhibited cytotoxicities similar to or moderately reduced when compared the high-affinity parental c-MET CAR-T cells in the 3 carcinoma cell lines tested. Although the cytotoxicities were significantly reduced in the 24 hour co-cultures compared to the high-affinity parental CAR-T cells, upon further incubation for 48 and 72 hours, the cytotoxicities typically approached the cytotoxicity levels obtained with the high-affinity parental CAR-T cells (see FIGS. 6-9 and Tables 6-9 below).

TABLE 6
In Vitro Cytotoxicity of Parental- and Variant Affy7-Based MET
CAR-T Cells Against SNU-638 Human Stomach Carcinoma Cells

| SNU638 | 0 hrs | stdev | 24 hrs | stdev | 48 hrs | stdev | 72 hrs | stdev |
|---|---|---|---|---|---|---|---|---|
| Affy7 | 0 | 0 | 78.6 | 7.1 | 95.9 | 1.9 | 99.5 | 0.3 |
| Affy7-Y6A | 0 | 0 | 79.2 | 5.8 | 93.5 | 3.3 | 98.7 | 0.4 |
| Affy7-Y6I | 0 | 0 | 67.0 | 3.9 | 83.6 | 2.4 | 93.2 | 0.7 |
| Affy7-Y6L | 0 | 0 | 66.9 | 2.2 | 84.5 | 2.8 | 93.5 | 0.3 |
| Affy7-Y6V | 0 | 0 | 68.1 | 8.2 | 81.9 | 5.4 | 93.2 | 2.1 |
| Affy7-Y6M | 0 | 0 | 61.2 | 3.0 | 77.5 | 1.8 | 90.6 | 0.4 |

TABLE 7
In Vitro Cytotoxicity of Parental- and Variant Affy7-Based MET
CAR-T Cells Against Hs746T Human Stomach Carcinoma Cells

| Hs746T | 0 hrs | stdev | 24 hrs | stdev | 48 hrs | stdev | 72 hrs | stdev |
|---|---|---|---|---|---|---|---|---|
| Affy7 | 0 | 0 | 87.1 | 3.1 | 97.6 | 0.7 | 99.2 | 0.3 |
| Affy7-Y6A | 0 | 0 | 88.3 | 4.9 | 98.4 | 0.8 | 98.8 | 0.6 |
| Affy7-Y6I | 0 | 0 | 1.5 | 2.3 | 45.4 | 5.6 | 69.4 | 7.9 |
| Affy7-Y6L | 0 | 0 | 1.6 | 12.2 | 42.9 | 9.3 | 64.5 | 10.9 |
| Affy7-Y6V | 0 | 0 | 65.0 | 2.8 | 94.8 | 0.8 | 98.2 | 0.2 |
| Affy7-Y6M | 0 | 0 | 47.6 | 2 | 87 | 4.8 | 96.4 | 0.4 |

TABLE 8
In Vitro Cytotoxicity of Parental- and Variant Affy7-Based MET CAR-T
Cells Against H1993 Non-Small Cell Lung Adenocarcinoma Cells

| H1993 | 0 hrs | stdev | 24 hrs | stdev | 48 hrs | stdev | 72 hrs | stdev |
|---|---|---|---|---|---|---|---|---|
| Affy7 | 0 | 0 | 85.6 | 0.6 | 97.7 | 0.8 | 99.6 | 0.5 |
| Affy7-Y6A | 0 | 0 | 85.7 | 6.7 | 96.9 | 0.4 | 99.2 | 0.2 |
| Affy7-Y6I | 0 | 0 | 11.7 | 8.9 | 45.8 | 5.9 | 73.5 | 1.4 |
| Affy7-Y6L | 0 | 0 | 12.8 | 9.9 | 42.6 | 4.4 | 71.2 | 4.8 |
| Affy7-Y6V | 0 | 0 | 40.0 | 7.5 | 84.2 | 2.2 | 93.8 | 1.3 |
| Affy7-Y6M | 0 | 0 | 21.3 | 7 | 70.5 | 1.1 | 89.6 | 1.1 |

TABLE 9
In Vitro Cytotoxicity of Parental- and Variant Affy6-Based MET CAR-T
Cells Against H1993 Non-Small Cell Lung Adenocarcinoma Cells

| H1993 | 0 hrs | stdev | 24 hrs | stdev | 48 hrs | stdev | 72 hrs | stdev |
|---|---|---|---|---|---|---|---|---|
| Affy6 | 0 | 0 | | | | | | |
| Affy6-I3A | 0 | 0 | 73.06 | 2.57 | 95.28 | 1.25 | 98.86 | 0.31 |
| Affy6-I3F | 0 | 0 | 64.67 | 2.71 | 91.32 | 1.73 | 96.99 | 0.21 |
| Affy6-I3G | 0 | 0 | 32.48 | 8.29 | 68.14 | 5.08 | 81.56 | 4.12 |
| Affy6-I3W | 0 | 0 | 74.88 | 5.65 | 93.5 | 0.88 | 98.20 | 0.24 |

### Example 4. Cytotoxicity of Reduced Affinity c-MET CAR-T Cells in Primary Cells

As implicitly suggested above, an ideal CAR-T effector cell would be potent enough to kill c-MET overexpressing cancer cells while sparing normal c-MET expressing cells. Therefore, normal human cells were utilized as target cells for examining cytotoxicity in co-cultures with the reduced affinity c-MET CAR-T cells. More specifically, in vitro cytotoxicity assays were carried out by co-culturing the reduced affinity c-MET CAR-T cells with normal primary cells from human epithelial lung, liver, kidney, and colon cells.

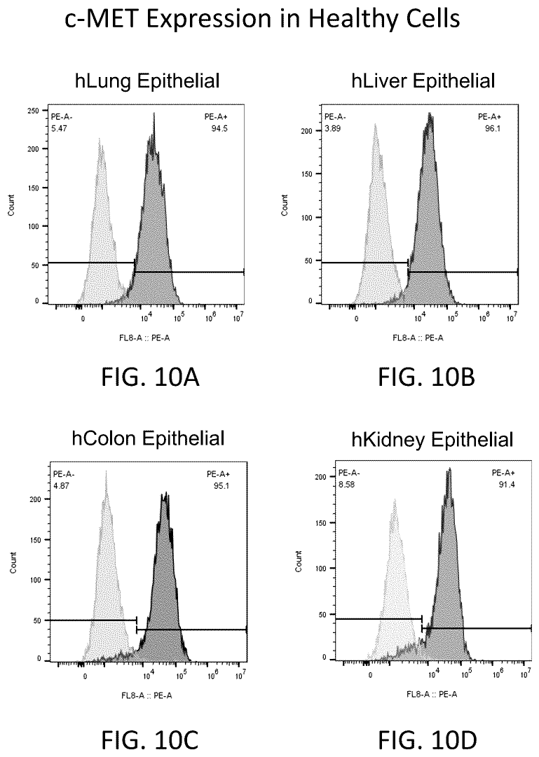

Initially, the normal primary cells were evaluated for c-MET expression by flow cytometry. Briefly, 10 μL of human HGFR/c-met PE-conjugated antibody (R&D Systems, FAB3582P) was added to 1×10⁶ cells in a 100 μL reaction volume containing 95 μL staining buffer (BioLegend, 420201). The resulting mixture was then incubated on ice for 30 min., washed with staining buffer and run on the flow cytometer. As shown in FIGS. 10A-10D, the results of this analysis confirm expression of c-MET in normal human epithelial cells.

Next, cytotoxicity assays were carried out by co-culturing normal primary target cells (human epithelial lung, colon, kidney, and liver) with c-met CAR-T cells at an effector to target ratio of 2:1. The co-cultures were maintained in T cell culture medium with no cytokine supplementation over a period of 48 hours. Cytotoxicity was measured using label-free cellular impedance to continuously monitor and record cell killing using 96-well electronic microplates with the xCELLigence Real-Time Cell Analysis (RTCA) SP instrument (Agilent).

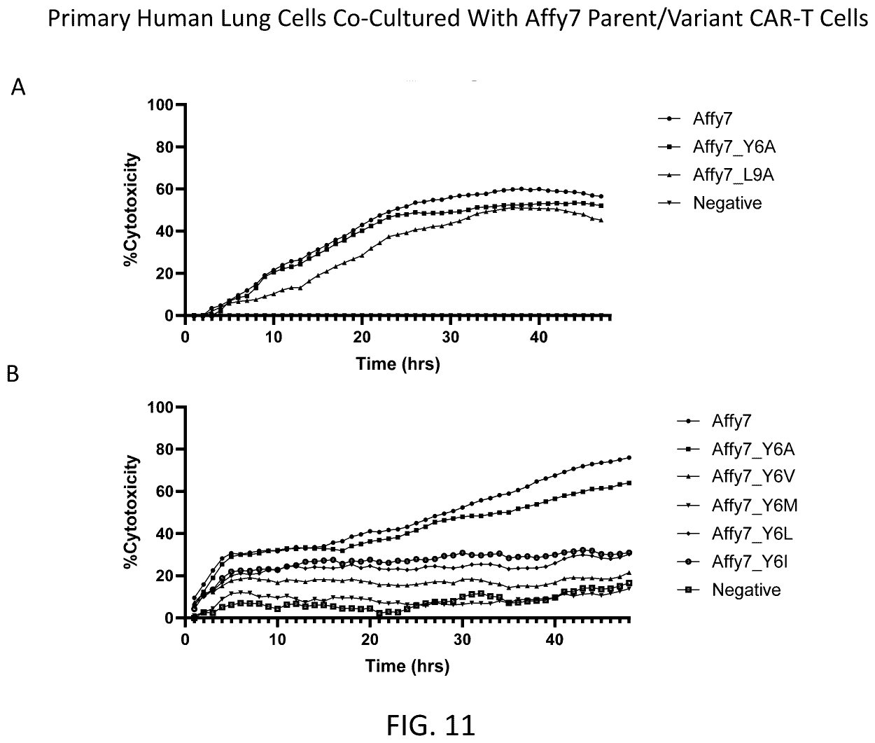

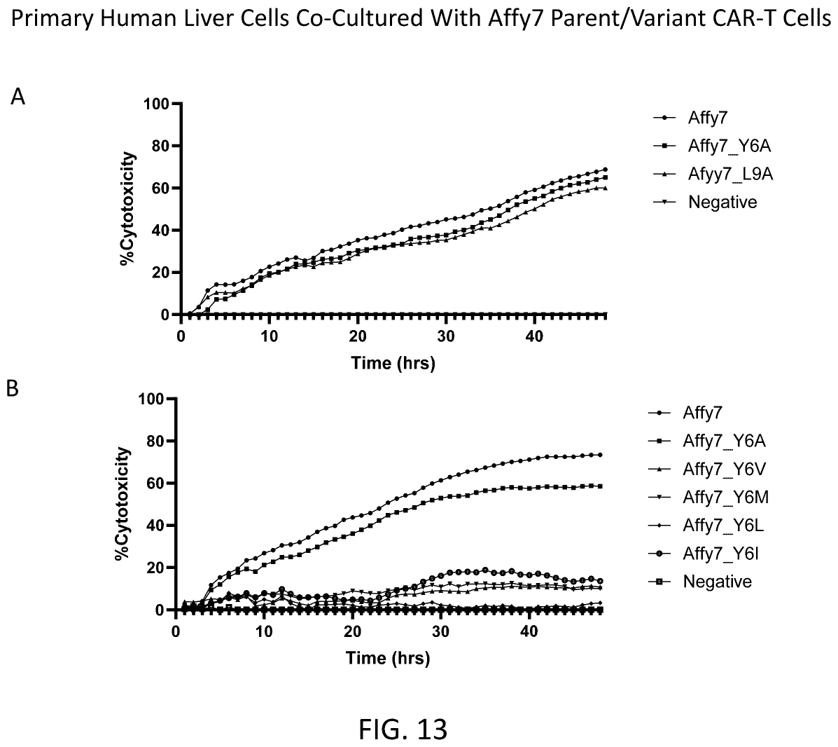

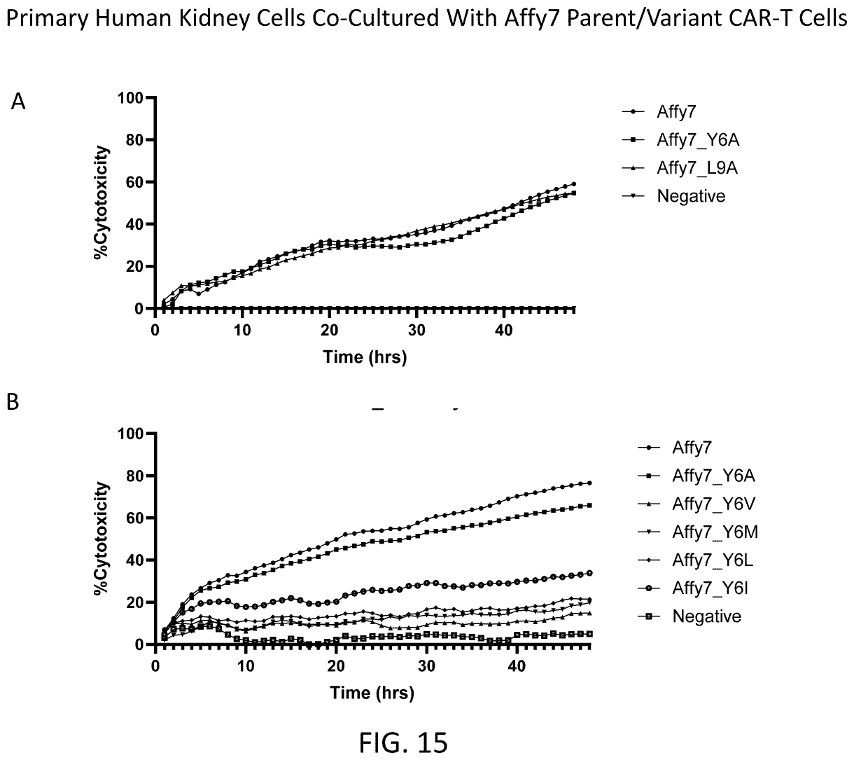

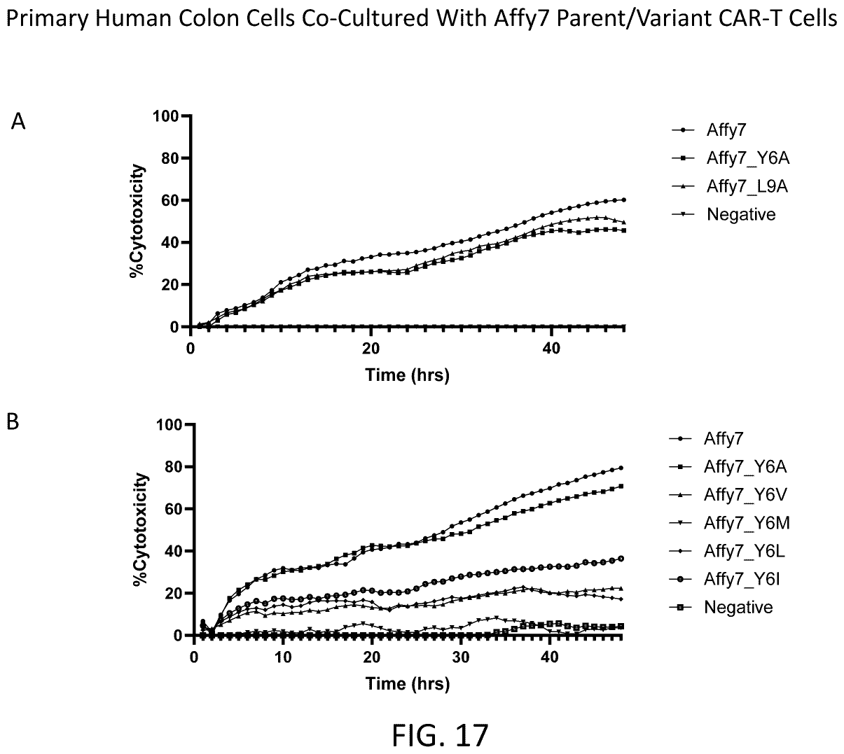

The results of this analysis are shown in Tables 10A-17B. The results show a correlation between the affinity of the binding between c-MET as shown in Tables 2-5 and cytotoxic activity. For example, among the Affy7 variants, Affy7-L9A and Affy7-Y6A CAR-T cells, which showed the least comparative reduction in binding affinity to c-MET (relative to the parental Affy7 CAR-T control cells) exhibited the greatest cytotoxicity against normal human lung (Tables 10A-10B; FIGS. 11A-11B), liver (Tables 12A-12B; FIGS. 13A-13B), kidney (Tables 14A-14B; FIGS. 15A-15B), and colon cells (Tables 16A-16B; FIGS. 17A-17B).

On the other hand, Affy7-Y6M CAR-T cells having a greater reduction in binding affinity to c-MET exhibited greatly reduced cytotoxicity toward normal human lung (Table 10B, FIG. 11B) and colon cells (Table 16B, FIG. 17B). Likewise, Affy7-Y6L CAR-T cells exhibited greatly reduced cytotoxicity toward normal human liver cells (Table 12B, FIG. 13B), while Affy7-Y6V CAR-T cells exhibited greatly reduced cytotoxicity toward normal human lung (Table 10B, FIG. 11B) and kidney cells (Table 14B, FIG. 15B).

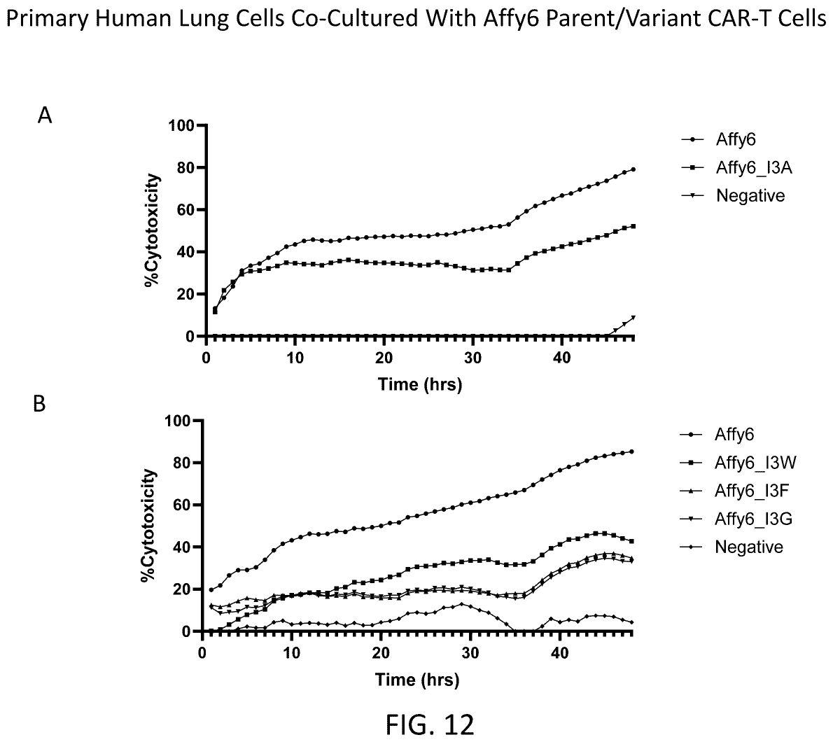

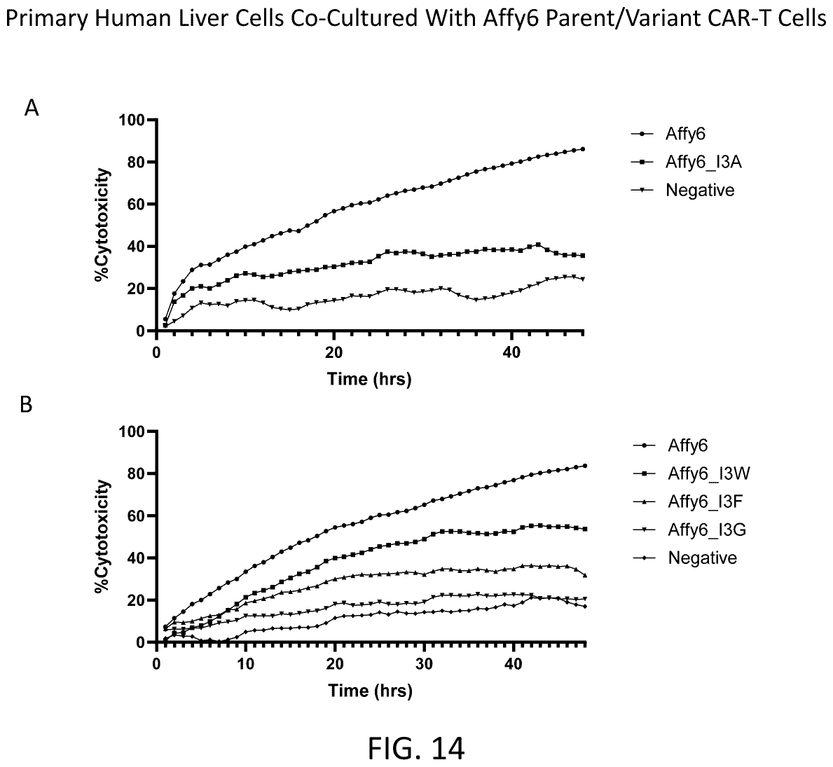

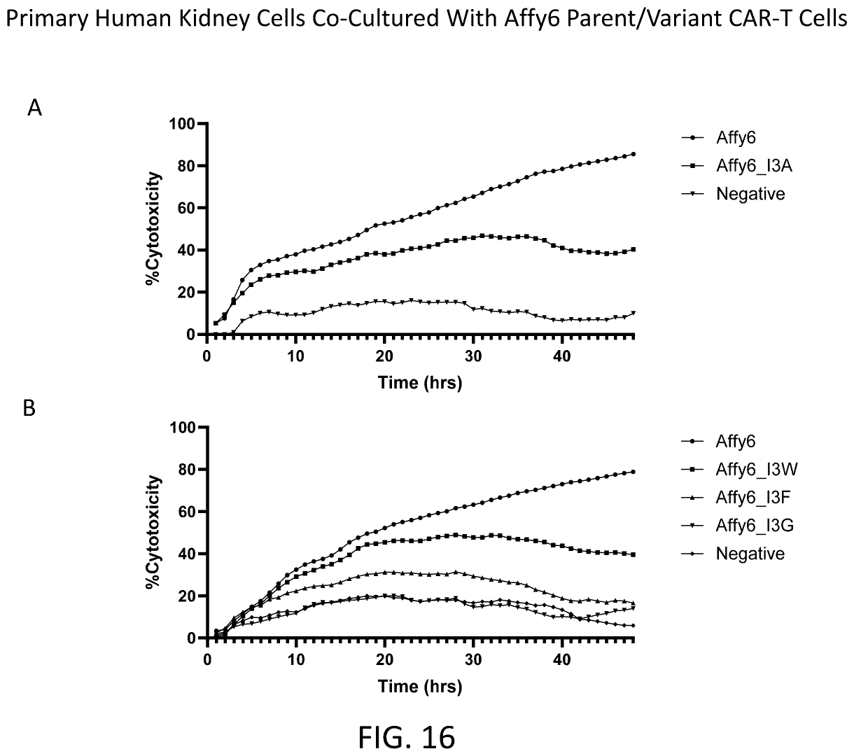

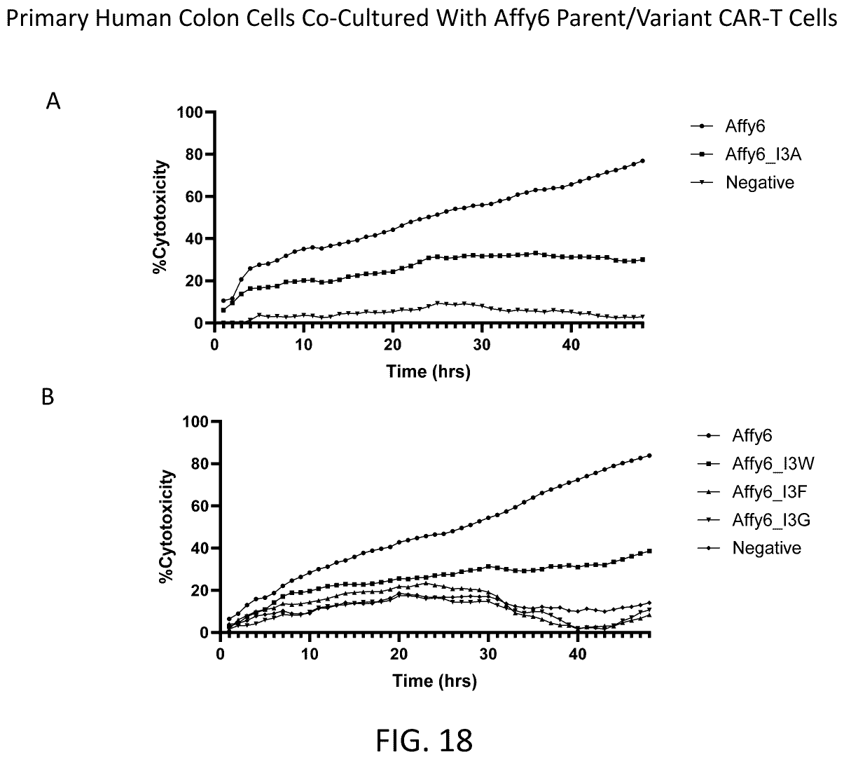

Among the Affy6 CAR-T cells variants, Affy6-I3F and I3G CAR-T cells were found to exhibit greatly reduced cytotoxicity toward normal human lung (Table 11B, FIG. 12B), liver (Table 13B, FIG. 14B), kidney (Table 15B, FIG. 16B), and colon cells (Table 17B, FIG. 18B).

TABLE 10A
Affy7 CAR-T Variants Co-cultured with Normal
Human Lung Cells (FIG. 11A, Round 1)

| Time (hrs) | Affy7 | Affy7_Y6A | Affy7_L9A | Negative |
|---|---|---|---|---|
| 8 | 14.7 | 13.1 | 7.5 | 0 |
| 16 | 33.3 | 31.3 | 20.9 | 0 |
| 24 | 50.7 | 47.6 | 38.3 | 0 |
| 32 | 57.1 | 50.0 | 46.4 | 0 |
| 40 | 59.9 | 53.0 | 50.7 | 0 |
| 47 | 56.5 | 52.1 | 45.3 | 0 |

TABLE 10B
Affy7 CAR-T Variants Co-cultured with Normal Human Lung Cells (FIG. 11A, Round 2)

| Time | | | | | | | |
|---|---|---|---|---|---|---|---|
| (hrs) | Affy7 | Affy7_Y6A | Affy7_Y6V | Affy7_Y6M | Affy7_Y6L | Affy7_Y6I | Negative |
| 8 | 31.8 | 30.6 | 18.2 | 9.9 | 21.1 | 23.1 | 6.7 |
| 16 | 35.7 | 32.6 | 18.0 | 9.7 | 23.6 | 27.5 | 5.4 |
| 24 | 43.2 | 40.1 | 15.4 | 6.3 | 22.8 | 26.9 | 4.1 |
| 32 | 55.8 | 48.3 | 17.9 | 6.9 | 25.4 | 29.7 | 11.6 |
| 40 | 67.5 | 56.5 | 16.7 | 9.5 | 25.9 | 29.9 | 9.7 |
| 48 | 76.0 | 64.0 | 21.5 | 13.8 | 30.1 | 30.9 | 16.5 |

TABLE 11A
Affy6 CAR-T Variants Co-cultured with Normal
Human Lung Cells (FIG. 12A Round 1)

| Time (hrs) | Affy6 | Affy6-I3A | Negative |
|---|---|---|---|
| 8 | 39.4 | 33.2 | 0 |
| 16 | 46.5 | 36.2 | 0 |
| 24 | 47.5 | 33.5 | 0 |
| 32 | 51.8 | 31.9 | 0 |
| 40 | 66.7 | 42.5 | 0 |
| 48 | 79.0 | 52.2 | 8.7 |

TABLE 11B
Affy6 CAR-T Variants Co-cultured with Normal
Human Lung Cells (FIG. 12B, Round 2)

| Time (hrs) | Affy6 | I3W | I3F | I3G | Negative |
|---|---|---|---|---|---|
| 8 | 38.4 | 14.3 | 17.1 | 14.8 | 4.3 |
| 16 | 47.1 | 20.9 | 16.4 | 17.3 | 2.6 |
| 24 | 54.7 | 30.6 | 19.1 | 19.5 | 8.9 |
| 32 | 63.1 | 34.0 | 18.0 | 18.4 | 8.8 |
| 40 | 76.4 | 41.2 | 29.4 | 28.0 | 4.3 |
| 48 | 85.2 | 42.7 | 34.8 | 33.1 | 4.2 |

TABLE 12A
Affy7 CAR-T Variants Co-Cultured with Normal
Human Liver Cells (FIG. 13A, Round 1)

| Time (hrs) | Affy7 | Affy7_Y6A | Affy7_L9A | Negative |
|---|---|---|---|---|
| 8 | 17.7 | 14.2 | 13.7 | 0 |
| 16 | 30.1 | 26.3 | 24.5 | 0 |
| 24 | 38.6 | 33.1 | 32.8 | 0 |
| 32 | 46.2 | 40.1 | 37.9 | 0 |
| 40 | 59.1 | 55.0 | 50.1 | 0 |
| 48 | 68.7 | 65.0 | 60.0 | 0 |

TABLE 12B
Affy7 CAR-T Variants Co-Cultured with Normal Human Liver Cells (FIG. 13B, Round 2)

| Time | | | | | | | |
|---|---|---|---|---|---|---|---|
| (hrs) | Affy7 | Affy7_Y6A | Affy7_Y6V | Affy7_Y6M | Affy7_Y6L | Affy7_Y6I | Negative |
| 8 | 23.4 | 19.3 | 6.5 | 7.2 | 6.1 | 7.8 | 0 |
| 16 | 36.9 | 29.8 | 3.9 | 6.2 | 2.3 | 6.2 | 0 |
| 24 | 50.7 | 44.8 | 5.5 | 9.0 | 2.5 | 7.4 | 0 |
| 32 | 64.0 | 53.9 | 8.7 | 10.9 | 1.1 | 17.8 | 0 |
| 40 | 71.2 | 57.5 | 10.7 | 11.3 | 0 | 16.4 | 0 |
| 48 | 73.4 | 58.5 | 10.8 | 10.0 | 3.2 | 13.6 | 0 |

TABLE 13A
Affy6 CAR-T Variants Co-Cultured with Normal
Human Liver Cells (FIG. 14A, Round 1)

| Time (hrs) | Affy6 | I3A | Negative |
|---|---|---|---|
| 8 | 36.0 | 23.9 | 11.8 |
| 16 | 47.2 | 28.2 | 10.3 |
| 24 | 60.8 | 32.7 | 16.2 |
| 32 | 69.8 | 35.7 | 20.0 |
| 40 | 79.3 | 38.4 | 18.0 |
| 48 | 86.0 | 35.5 | 24.3 |

TABLE 13B
Affy6 CAR-T Variants Co-Cultured with Normal
Human Liver Cells (FIG. 14B, Round 2)

| Time (hrs) | Affy6 | I3W | I3F | I3G | Negative |
|---|---|---|---|---|---|
| 8 | 28.4 | 15.1 | 15.3 | 9.6 | 1.2 |
| 16 | 47.1 | 32.4 | 24.7 | 13.7 | 7.1 |
| 24 | 59.0 | 44.0 | 31.8 | 18.1 | 13.0 |
| 32 | 67.9 | 52.5 | 34.8 | 22.3 | 14.9 |
| 40 | 76.8 | 52.4 | 34.8 | 22.5 | 17.3 |
| 48 | 83.6 | 53.6 | 31.8 | 20.5 | 17.0 |

TABLE 14A
Affy7 CAR-T Variants Co-Cultured with Normal
Human Kidney Cells (FIG. 15A, Round 1)

| Time (hrs) | Affy7 | Affy7_Y6A | Affy7_L9A | Negative |
|---|---|---|---|---|
| 8 | 12.5 | 15.8 | 12.9 | 0 |
| 16 | 27.2 | 27.1 | 23.9 | 0 |
| 24 | 32.4 | 29.4 | 30.7 | 0 |
| 32 | 36.9 | 31.1 | 38.8 | 0 |
| 40 | 47.4 | 42.6 | 46.9 | 0 |
| 48 | 59.0 | 54.7 | 54.7 | 0 |

TABLE 14B
Affy7 CAR-T Variants Co-Cultured with Normal
Human Kidney Cells (FIG. 15B, Round 2)

| Time | | | | | | | |
|---|---|---|---|---|---|---|---|
| (hrs) | Affy7 | Affy7_Y6A | Affy7_Y6V | Affy7_Y6M | Affy7_Y6L | Affy7_Y6I | Negative |
| 8 | 32.7 | 29.2 | 9.3 | 8.8 | 11.7 | 20.5 | 4.6 |
| 16 | 43.5 | 39.3 | 9.7 | 10.0 | 12.9 | 20.9 | 2.1 |
| 24 | 53.8 | 48.9 | 10.6 | 12.0 | 14.8 | 25.8 | 3.7 |
| 32 | 61.2 | 53.9 | 10.3 | 13.3 | 16.2 | 27.6 | 4.2 |
| 40 | 70.2 | 60.5 | 11.0 | 15.9 | 17.1 | 29.7 | 4.4 |
| 48 | 76.5 | 65.9 | 14.8 | 19.7 | 21.4 | 33.9 | 4.9 |

TABLE 15A
Affy6 CAR-T Variants Co-Cultured with Normal
Human Kidney Cells (FIG. 16A, Round 1)

| Time (hrs) | Affy6 | I3A | Negative |
|---|---|---|---|
| 8 | 35.5 | 28.0 | 9.7 |
| 16 | 45.2 | 35.0 | 14.5 |
| 24 | 56.9 | 41.0 | 15.4 |
| 32 | 68.9 | 46.5 | 11.0 |
| 40 | 78.5 | 41.0 | 6.5 |
| 48 | 85.5 | 40.2 | 10.0 |

TABLE 15B
Affy6 CAR-T Variants Co-Cultured with Normal
Human Kidney Cells (FIG. 16B, Round 2)

| Time (hrs) | Affy6 | I3W | I3F | I3G | Negative |
|---|---|---|---|---|---|
| 8 | 25.7 | 23.6 | 19.2 | 10.1 | 12.2 |
| 16 | 45.4 | 39.4 | 28.0 | 17.9 | 18.8 |
| 24 | 57.0 | 46.0 | 30.4 | 17.2 | 17.3 |
| 32 | 65.5 | 48.6 | 27.8 | 15.8 | 17.1 |
| 40 | 73.0 | 43.6 | 18.8 | 10.2 | 13.4 |
| 48 | 78.8 | 39.5 | 16.5 | 14.0 | 5.9 |

TABLE 16A
Affy7 CAR-T Variants Co-Cultured with Normal
Human Colon Cells (FIG. 17A, Round 1)

| Time (hrs) | Affy7 | Affy7_Y6A | Affy7_L9A | Negative |
|---|---|---|---|---|
| 8 | 13.8 | 13.1 | 12.1 | 0 |
| 16 | 29.3 | 25.0 | 25.0 | 0 |
| 24 | 34.9 | 25.6 | 27.2 | 0 |
| 32 | 42.8 | 35.8 | 38.2 | 0 |
| 40 | 54.1 | 45.4 | 48.5 | 0 |
| 48 | 60.1 | 45.6 | 49.6 | 0 |

TABLE 16B
Affy7 CAR-T Variants Co-Cultured with Normal Human Colon Cells (FIG. 17B, Round 2)

| Time | | | | | | | |
|---|---|---|---|---|---|---|---|
| (hrs) | Affy7 | Affy7_Y6A | Affy7_Y6V | Affy7_Y6M | Affy7_Y6L | Affy7_Y6I | Negative |
| 8 | 28.5 | 26.5 | 9.4 | 1.4 | 12.1 | 15.1 | 0 |
| 16 | 33.9 | 36.3 | 13.5 | 1.7 | 16.4 | 18.8 | 0 |
| 24 | 43.2 | 42.5 | 14.0 | 1.4 | 13.6 | 20.8 | 0 |
| 32 | 57.0 | 51.5 | 18.2 | 7.1 | 19.0 | 28.9 | 0 |
| 40 | 69.7 | 62.7 | 20.3 | 1.8 | 20.0 | 32.5 | 5.4 |
| 48 | 79.4 | 70.7 | 22.2 | 3.6 | 17.2 | 36.4 | 4.3 |

TABLE 17A
Affy6 CAR-T Variants Co-Cultured with Normal
Human Colon Cells (FIG. 18A, Round 1)

| Time (hrs) | Affy6 | I3A | Negative |
|---|---|---|---|
| 8 | 31.9 | 19.4 | 2.7 |
| 16 | 39.2 | 22.5 | 4.4 |
| 24 | 50.3 | 30.9 | 7.8 |
| 32 | 57.8 | 31.8 | 6.2 |
| 40 | 65.7 | 31.1 | 5.2 |
| 48 | 76.8 | 30.0 | 2.9 |

TABLE 17B
Affy6 CAR-T Variants Co-Cultured with Normal
Human Colon Cells (FIG. 18B, Round 2)

| Time (hrs) | Affy6 | I3W | I3F | I3G | Negative |
|---|---|---|---|---|---|
| 8 | 24.6 | 18.8 | 13.2 | 8.2 | 8.9 |
| 16 | 37.7 | 22.7 | 19.2 | 14.4 | 13.7 |
| 24 | 46.4 | 27.0 | 22.4 | 16.4 | 16.6 |
| 32 | 57.3 | 29.9 | 13.3 | 11.4 | 13.6 |
| 40 | 72.3 | 30.9 | 1.6 | 2.0 | 10.0 |
| 48 | 83.8 | 38.6 | 8.3 | 10.7 | 14.1 |

### LIST OF SEQUENCES

Exemplary amino acid sequences described herein are provided in Table 18 below:

TABLE 18
Amino Acid and Nucleotide Sequences for Exemplary Polypeptides Described
Herein

| SEQ ID | | |
|---|---|---|
| NO: | Name | Sequence |
| 1 | Affy7 HCDR1 | SGYTFTSY |
| 2 | Affy7 HCDR2 | LEWIGMIDPSNSDTRFN |
| 3 | Affy7-Y6A CDR3 | AGSYVSPLDY |
| 4 | Affy7-L9A HCDR3 | YGSYVSPADY |
| 5 | Affy7-Y6V HCDR3 | VGSYVSPLDY |
| 6 | Affy7-Y6L HCDR3 | LGSYVSPLDY |
| 7 | Affy7-Y6I HCDR3 | IGSYVSPLDY |
| 8 | Affy7-Y6M HCDR3 | MGSYVSPLDY |
| 9 | Affy7 LCDR1 | QSLLYTSSQKN |
| 10 | Affy7 LCDR2 | PKLLIYWASTRE |
| 11 | Affy7 LCDR3 | YYAYP |
| 12 | Affy7 VH (wt) | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATYGSYVSPLDYWG |
| | | QGTSVTVSS |
| 13 | Affy7-Y6A VH* | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATAGSYVSPLDYWG |
| | | QGTSVTVSS |
| 14 | Affy7-L9A VH | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATYGSYVSPADYWG |
| | | QGTSVTVSS |
| 15 | Affy7-Y6V VH | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATVGSYVSPLDYWG |
| | | QGTSVTVSS |
| 16 | Affy7-Y6L VH | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATLGSYVSPLDYWG |
| | | QGTSVTVSS |
| 17 | Affy7-Y6I VH | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATIGSYVSPLDYWGQ |
| | | GTSVTVSS |
| 18 | Affy7-Y6M VH | QVQLQQSGPELVRPGASVKMSCRASGYTFTSYWLHW |
| | | VKQRPGQGLEWIGMIDPSNSDTFNPNFKDKATLNVD |
| | | RSSNTAYMLLSSLTSADSAVYYCATMGSYVSPLDYWG |
| | | QGTSVTVSS |
| 19 | Affy7 VL (wt) | DIMMSQSPSSLTVSVGEKVTVSCKSSQSLLYTSSQKNYL |
| | | AWYQQKPGQSPKLLIYWASTRESGVPDRFTGSGSGTDF |
| | | TLTITSVKADDLAVYYCQQYYAYPWTFGGGTKLEIKR |
| 20 | Affy7 VH (wt) | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACTG |
| | | GGTCAAGCAGAGGCCCGGACAGGGTCTGGAGTGGA |
| | | TCGGTATGATTGACCCGTCAAATAGTGACACCCGCTTC |
| | | AACCCCAACTTCAAGGACAAGGCCACTCTCAACGTG |
| | | GACCGCTCGAGCAACACGGCCTACATGCTGCTGTCC |
| | | TCTCTGACCTCGGCGGACAGCGCCGTGTACTACTGT |
| | | GCCACCTACGGCTCCTACGTGAGCCCTTTGGATTATTG |
| | | GGGCCAGGGCACCTCCGTCACCGTGTCCTCC |
| 21 | Affy7-Y6A VH | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACTG |
| | | GGTCAAGCAGAGGCCCGGACAGGGTCTGGAGTGGA |
| | | TCGGTATGATTGACCCGTCAAATAGTGACACCCGCTTC |
| | | AACCCCAACTTCAAGGACAAGGCCACTCTCAACGTG |
| | | GACCGCTCGAGCAACACGGCCTACATGCTGCTGTCC |
| | | TCTCTGACCTCGGCGGACAGCGCCGTGTACTACTGT |
| | | GCCACCGCGGGCTCCTACGTGAGCCCTTTGGATTATT |
| | | GGGGCCAGGGCACCTCCGTCACCGTGTCCTCC |
| 22 | Affy7-L9A VH | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACTG |
| | | GGTCAAGCAGAGGCCCGGACAGGGTCTGGAGTGGA |
| | | TCGGTATGATTGACCCGTCAAATAGTGACACCCGCTTC |
| | | AACCCCAACTTCAAGGACAAGGCCACTCTCAACGTG |
| | | GACCGCTCGAGCAACACGGCCTACATGCTGCTGTCC |
| | | TCTCTGACCTCGGCGGACAGCGCCGTGTACTACTGT |
| | | GCCACCTACGGCTCCTACGTGAGCCCTGCGGATTATT |
| | | GGGGCCAGGGCACCTCCGTCACCGTGTCCTCC |
| 23 | Affy7-Y6V VH | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACTG |
| | | GGTCAAGCAGAGGCCCGGACAGGGTCTGGAGTGGA |
| | | TCGGTATGATTGACCCGTCAAATAGTGACACCCGCTTC |
| | | AACCCCAACTTCAAGGACAAGGCCACTCTCAACGTG |
| | | GACCGCTCGAGCAACACGGCCTACATGCTGCTGTCC |
| | | TCTCTGACCTCGGCGGACAGCGCCGTGTACTACTGT |
| | | GCCACCGTCGGCTCCTACGTGAGCCCTTTGGATTATT |
| | | GGGGCCAGGGCACCTCCGTCACCGTGTCCTCC |
| 24 | Affy7-Y6L VH | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACTG |
| | | GGTCAAGCAGAGGCCCGGACAGGGTCTGGAGTGGA |
| | | TCGGTATGATTGACCCGTCAAATAGTGACACCCGCTTC |
| | | AACCCCAACTTCAAGGACAAGGCCACTCTCAACGTG |
| | | GACCGCTCGAGCAACACGGCCTACATGCTGCTGTCC |
| | | TCTCTGACCTCGGCGGACAGCGCCGTGTACTACTGT |
| | | GCCACCCTCGGCTCCTACGTGAGCCCTTTGGATTATTG |
| | | GGGCCAGGGCACCTCCGTCACCGTGTCCTCC |
| 25 | Affy7-Y6I VH | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACTG |
| | | GGTCAAGCAGAGGCCCGGACAGGGTCTGGAGTGGA |
| | | TCGGTATGATTGACCCGTCAAATAGTGACACCCGCTTC |
| | | AACCCCAACTTCAAGGACAAGGCCACTCTCAACGTG |
| | | GACCGCTCGAGCAACACGGCCTACATGCTGCTGTCC |
| | | TCTCTGACCTCGGCGGACAGCGCCGTGTACTACTGT |
| | | GCCACCATCGGCTCCTACGTGAGCCCTTTGGATTATTG |
| | | GGGCCAGGGCACCTCCGTCACCGTGTCCTCC |
| 26 | Affy7-Y6M VH | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACTG |
| | | GGTCAAGCAGAGGCCCGGACAGGGTCTGGAGTGGA |
| | | TCGGTATGATTGACCCGTCAAATAGTGACACCCGCTTC |
| | | AACCCCAACTTCAAGGACAAGGCCACTCTCAACGTG |
| | | GACCGCTCGAGCAACACGGCCTACATGCTGCTGTCC |
| | | TCTCTGACCTCGGCGGACAGCGCCGTGTACTACTGT |
| | | GCCACCATGGGCTCCTACGTGAGCCCTTTGGATTATT |
| | | GGGGCCAGGGCACCTCCGTCACCGTGTCCTCC |
| 27 | Affy7 VL | GACATCATGATGTCTCAGAGTCCCTCTTCTCTGACC |
| | | GTGTCCGTGGGCGAGAAGGTGACCGTGTCATGCAA |
| | | GTCCTCACAGAGCCTTCTGTACACCAGCTCCCAGAAG |
| | | AACTACCTGGCGTGGTACCAGCAGAAGCCCGGACA |
| | | GAGCCCCAAGCTGCTGATCTATTGGGCTTCCACCCGC |
| | | GAGAGCGGCGTCCCCGACCGCTTCACCGGCTCCGGC |
| | | TCCGGGACCGACTTCACCCTGACCATCACCTCCGTG |
| | | AAGGCCGATGACCTGGCCGTGTACTACTGTCAACA |
| | | GTATTACGCCTACCCGTGGACCTTCGGAGGCGGCAC |
| | | CAAGCTGGAGATCAAGCGC |
| 28 | Affy7 scFv (control) | QVQLQQSGPELVRPGASVKMSCRASGYTFTSYWLHW |
| | | VKQRPGQGLEWIGMIDPSNSDTRENPNFKDKATLNVD |
| | | RSSNTAYMLLSSLTSADSAVYYCATYGSYVSPLDYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKR |
| 29 | Affy7-Y6A scFv | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATAGSYVSPLDYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKR |
| 30 | Affy7-L9A scFv | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATYGSYVSPADYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKR |
| 31 | Affy7-Y6V scFv | QVQLQQSGPELVRPGASVKMSCRASGYTFTSYWLHW |
| | | VKQRPGQGLEWIGMIDPSNSDTRENPNFKDKATLNVD |
| | | RSSNTAYMLLSSLTSADSAVYYCATVGSYVSPLDYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKR |
| 32 | Affy7-Y6L scFv | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATLGSYVSPLDYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKR |
| 33 | Affy7-Y6I scFv | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATIGSYVSPLDYWGQ |
| | | GTSVTVSSGGGGSGGGGSGGGGSDIMMSQSPSSLTVS |
| | | VGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKR |
| 34 | Affy7-Y6M scFv | QVQLQQSGPELVRPGASVKMSCRASGYTFTSYWLHW |
| | | VKQRPGQGLEWIGMIDPSNSDTRENPNFKDKATLNVD |
| | | RSSNTAYMLLSSLTSADSAVYYCATMGSYVSPLDYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKR |
| 35 | Affy7 scFv (control) | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCTACGGCTCCTACGTGAGCCCTTTGGATT |
| | | ATTGGGGCCAGGGCACCTCCGTCACCGTGTCCTCCG |
| | | GTGGAGGCGGTTCAGGCGGAGGTGGCTCTGGCGGT |
| | | GGCGGATCGGACATCATGATGTCTCAGAGTCCCTCT |
| | | TCTCTGACCGTGTCCGTGGGCGAGAAGGTGACCGT |
| | | GTCATGCAAGTCCTCACAGAGCCTTCTGTACACCAG |
| | | CTCCCAGAAGAACTACCTGGCGTGGTACCAGCAGA |
| | | AGCCCGGACAGAGCCCCAAGCTGCTGATCTATTGG |
| | | GCTTCCACCCGCGAGAGCGGCGTCCCCGACCGCTTC |
| | | ACCGGCTCCGGCTCCGGGACCGACTTCACCCTGACC |
| | | ATCACCTCCGTGAAGGCCGATGACCTGGCCGTGTAC |
| | | TACTGTCAACAGTATTACGCCTACCCGTGGACCTTC |
| | | GGAGGCGGCACCAAGCTGGAGATCAAGCGC |
| 36 | Affy7-Y6A scFv | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCGCGGGCTCCTACGTGAGCCCTTTGGAT |
| | | TATTGGGGCCAGGGCACCTCCGTCACCGTGTCCTCC |
| | | GGTGGAGGCGGTTCAGGCGGAGGTGGCTCTGGCGG |
| | | TGGCGGATCGGACATCATGATGTCTCAGAGTCCCTC |
| | | TTCTCTGACCGTGTCCGTGGGCGAGAAGGTGACCGT |
| | | GTCATGCAAGTCCTCACAGAGCCTTCTGTACACCAG |
| | | CTCCCAGAAGAACTACCTGGCGTGGTACCAGCAGA |
| | | AGCCCGGACAGAGCCCCAAGCTGCTGATCTATTGG |
| | | GCTTCCACCCGCGAGAGCGGCGTCCCCGACCGCTTC |
| | | ACCGGCTCCGGCTCCGGGACCGACTTCACCCTGACC |
| | | ATCACCTCCGTGAAGGCCGATGACCTGGCCGTGTAC |
| | | TACTGTCAACAGTATTACGCCTACCCGTGGACCTTC |
| | | GGAGGCGGCACCAAGCTGGAGATCAAGCGC |
| 37 | Affy7-L9A scFv | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCTACGGCTCCTACGTGAGCCCTGCGGAT |
| | | TATTGGGGCCAGGGCACCTCCGTCACCGTGTCCTCC |
| | | GGTGGAGGCGGTTCAGGCGGAGGTGGCTCTGGCGG |
| | | TGGCGGATCGGACATCATGATGTCTCAGAGTCCCTC |
| | | TTCTCTGACCGTGTCCGTGGGCGAGAAGGTGACCGT |
| | | GTCATGCAAGTCCTCACAGAGCCTTCTGTACACCAG |
| | | CTCCCAGAAGAACTACCTGGCGTGGTACCAGCAGA |
| | | AGCCCGGACAGAGCCCCAAGCTGCTGATCTATTGG |
| | | GCTTCCACCCGCGAGAGCGGCGTCCCCGACCGCTTC |
| | | ACCGGCTCCGGCTCCGGGACCGACTTCACCCTGACC |
| | | ATCACCTCCGTGAAGGCCGATGACCTGGCCGTGTAC |
| | | TACTGTCAACAGTATTACGCCTACCCGTGGACCTTC |
| | | GGAGGCGGCACCAAGCTGGAGATCAAGCGC |
| 38 | Affy7-Y6V scFv | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCGTCGGCTCCTACGTGAGCCCTTTGGAT |
| | | TATTGGGGCCAGGGCACCTCCGTCACCGTGTCCTCC |
| | | GGTGGAGGCGGTTCAGGCGGAGGTGGCTCTGGCGG |
| | | TGGCGGATCGGACATCATGATGTCTCAGAGTCCCTC |
| | | TTCTCTGACCGTGTCCGTGGGCGAGAAGGTGACCGT |
| | | GTCATGCAAGTCCTCACAGAGCCTTCTGTACACCAG |
| | | CTCCCAGAAGAACTACCTGGCGTGGTACCAGCAGA |
| | | AGCCCGGACAGAGCCCCAAGCTGCTGATCTATTGG |
| | | GCTTCCACCCGCGAGAGCGGCGTCCCCGACCGCTTC |
| | | ACCGGCTCCGGCTCCGGGACCGACTTCACCCTGACC |
| | | ATCACCTCCGTGAAGGCCGATGACCTGGCCGTGTAC |
| | | TACTGTCAACAGTATTACGCCTACCCGTGGACCTTC |
| | | GGAGGCGGCACCAAGCTGGAGATCAAGCGC |
| 39 | Affy7-Y6L scFv | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCCTCGGCTCCTACGTGAGCCCTTTGGAT |
| | | TATTGGGGCCAGGGCACCTCCGTCACCGTGTCCTCC |
| | | GGTGGAGGCGGTTCAGGCGGAGGTGGCTCTGGCGG |
| | | TGGCGGATCGGACATCATGATGTCTCAGAGTCCCTC |
| | | TTCTCTGACCGTGTCCGTGGGCGAGAAGGTGACCGT |
| | | GTCATGCAAGTCCTCACAGAGCCTTCTGTACACCAG |
| | | CTCCCAGAAGAACTACCTGGCGTGGTACCAGCAGA |
| | | AGCCCGGACAGAGCCCCAAGCTGCTGATCTATTGG |
| | | GCTTCCACCCGCGAGAGCGGCGTCCCCGACCGCTTC |
| | | ACCGGCTCCGGCTCCGGGACCGACTTCACCCTGACC |
| | | ATCACCTCCGTGAAGGCCGATGACCTGGCCGTGTAC |
| | | TACTGTCAACAGTATTACGCCTACCCGTGGACCTTC |
| | | GGAGGCGGCACCAAGCTGGAGATCAAGCGC |
| 40 | Affy7-Y6I scFv | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCATCGGCTCCTACGTGAGCCCTTTGGAT |
| | | TATTGGGGCCAGGGCACCTCCGTCACCGTGTCCTCC |
| | | GGTGGAGGCGGTTCAGGCGGAGGTGGCTCTGGCGG |
| | | TGGCGGATCGGACATCATGATGTCTCAGAGTCCCTC |
| | | TTCTCTGACCGTGTCCGTGGGCGAGAAGGTGACCGT |
| | | GTCATGCAAGTCCTCACAGAGCCTTCTGTACACCAG |
| | | CTCCCAGAAGAACTACCTGGCGTGGTACCAGCAGA |
| | | AGCCCGGACAGAGCCCCAAGCTGCTGATCTATTGG |
| | | GCTTCCACCCGCGAGAGCGGCGTCCCCGACCGCTTC |
| | | ACCGGCTCCGGCTCCGGGACCGACTTCACCCTGACC |
| | | ATCACCTCCGTGAAGGCCGATGACCTGGCCGTGTAC |
| | | TACTGTCAACAGTATTACGCCTACCCGTGGACCTTC |
| | | GGAGGCGGCACCAAGCTGGAGATCAAGCGC |
| 41 | Affy7-Y6M scFv | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCATGGGCTCCTACGTGAGCCCTTTGGAT |
| | | TATTGGGGCCAGGGCACCTCCGTCACCGTGTCCTCC |
| | | GGTGGAGGCGGTTCAGGCGGAGGTGGCTCTGGCGG |
| | | TGGCGGATCGGACATCATGATGTCTCAGAGTCCCTC |
| | | TTCTCTGACCGTGTCCGTGGGCGAGAAGGTGACCGT |
| | | GTCATGCAAGTCCTCACAGAGCCTTCTGTACACCAG |
| | | CTCCCAGAAGAACTACCTGGCGTGGTACCAGCAGA |
| | | AGCCCGGACAGAGCCCCAAGCTGCTGATCTATTGG |
| | | GCTTCCACCCGCGAGAGCGGCGTCCCCGACCGCTTC |
| | | ACCGGCTCCGGCTCCGGGACCGACTTCACCCTGACC |
| | | ATCACCTCCGTGAAGGCCGATGACCTGGCCGTGTAC |
| | | TACTGTCAACAGTATTACGCCTACCCGTGGACCTTC |
| | | GGAGGCGGCACCAAGCTGGAGATCAAGCGC |
| 42 | Affy7 CAR (control) | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATYGSYVSPLDYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKRASTTTPAPRPP |
| | | TPAPTIASQPLSLRPEACRPAAGGAVHTRGLDFACDF |
| | | WVLVVVGGVLACYSLLVTVAFIIFWVRSKRSRLLHSD |
| | | YMNMTPRRPGPTRKHYQPYAPPRDFAAYRSLRVKFS |
| | | RSADAPAYQQGQNQLYNELNLGRREEYDVLDKRRGR |
| | | DPEMGGKPRRKNPQEGLYNELQKDKMAEAYSEIGMK |
| | | GERRRGKGHDGLYQGLSTATKDTYDALHMQALPPR |
| 43 | Affy7-Y6A CAR | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATAGSYVSPLDYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKRASTTTPAPRPP |
| | | TPAPTIASQPLSLRPEACRPAAGGAVHTRGLDFACDF |
| | | WVLVVVGGVLACYSLLVTVAFIIFWVRSKRSRLLHSD |
| | | YMNMTPRRPGPTRKHYQPYAPPRDFAAYRSLRVKFS |
| | | RSADAPAYQQGQNQLYNELNLGRREEYDVLDKRRGR |
| | | DPEMGGKPRRKNPQEGLYNELQKDKMAEAYSEIGMK |
| | | GERRRGKGHDGLYQGLSTATKDTYDALHMQALPPR |
| 44 | Affy7-L9A CAR | QVQLQQSGPELVRPGASVKMSCRASGYTFTSYWLHW |
| | | VKQRPGQGLEWIGMIDPSNSDTRENPNFKDKATLNVD |
| | | RSSNTAYMLLSSLTSADSAVYYCATYGSYVSPADYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKRASTTTPAPRPP |
| | | TPAPTIASQPLSLRPEACRPAAGGAVHTRGLDFACDF |
| | | WVLVVVGGVLACYSLLVTVAFIIFWVRSKRSRLLHSD |
| | | YMNMTPRRPGPTRKHYQPYAPPRDFAAYRSLRVKFS |
| | | RSADAPAYQQGQNQLYNELNLGRREEYDVLDKRRGR |
| | | DPEMGGKPRRKNPQEGLYNELQKDKMAEAYSEIGMK |
| | | GERRRGKGHDGLYQGLSTATKDTYDALHMQALPPR |
| 45 | Affy7-Y6V CAR | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATVGSYVSPLDYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKRASTTTPAPRPP |
| | | TPAPTIASQPLSLRPEACRPAAGGAVHTRGLDFACDF |
| | | WVLVVVGGVLACYSLLVTVAFIIFWVRSKRSRLLHSD |
| | | YMNMTPRRPGPTRKHYQPYAPPRDFAAYRSLRVKFS |
| | | RSADAPAYQQGQNQLYNELNLGRREEYDVLDKRRGR |
| | | DPEMGGKPRRKNPQEGLYNELQKDKMAEAYSEIGMK |
| | | GERRRGKGHDGLYQGLSTATKDTYDALHMQALPPR |
| 46 | Affy7-Y6L CAR | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATLGSYVSPLDYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKRASTTTPAPRPP |
| | | TPAPTIASQPLSLRPEACRPAAGGAVHTRGLDFACDF |
| | | WVLVVVGGVLACYSLLVTVAFIIFWVRSKRSRLLHSD |
| | | YMNMTPRRPGPTRKHYQPYAPPRDFAAYRSLRVKFS |
| | | RSADAPAYQQGQNQLYNELNLGRREEYDVLDKRRGR |
| | | DPEMGGKPRRKNPQEGLYNELQKDKMAEAYSEIGMK |
| | | GERRRGKGHDGLYQGLSTATKDTYDALHMQALPPR |
| 47 | Affy7-Y6I CAR | QVQLQQSGPELVRPGASVKMSCRASGYTFTSYWLHW |
| | | VKQRPGQGLEWIGMIDPSNSDTRENPNFKDKATLNVD |
| | | RSSNTAYMLLSSLTSADSAVYYCATIGSYVSPLDYWGQ |
| | | GTSVTVSSGGGGSGGGGSGGGGSDIMMSQSPSSLTVS |
| | | VGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKRASTTTPAPRPP |
| | | TPAPTIASQPLSLRPEACRPAAGGAVHTRGLDFACDF |
| | | WVLVVVGGVLACYSLLVTVAFIIFWVRSKRSRLLHSD |
| | | YMNMTPRRPGPTRKHYQPYAPPRDFAAYRSLRVKFS |
| | | RSADAPAYQQGQNQLYNELNLGRREEYDVLDKRRGR |
| | | DPEMGGKPRRKNPQEGLYNELQKDKMAEAYSEIGMK |
| | | GERRRGKGHDGLYQGLSTATKDTYDALHMQALPPR |
| 48 | Affy7-Y6M CAR | QVQLQQSGPELVRPGASVKMSCRASGYTFTSYWLHW |
| | | VKQRPGQGLEWIGMIDPSNSDTRFNPNFKDKATLNVD |
| | | RSSNTAYMLLSSLTSADSAVYYCATMGSYVSPLDYWG |
| | | QGTSVTVSSGGGGSGGGGSGGGGSDIMMSQSPSSLTV |
| | | SVGEKVTVSCKSSQSLLYTSSQKNYLAWYQQKPGQSP |
| | | KLLIYWASTRESGVPDRFTGSGSGTDFTLTITSVKADD |
| | | LAVYYCQQYYAYPWTFGGGTKLEIKRASTTTPAPRPP |
| | | TPAPTIASQPLSLRPEACRPAAGGAVHTRGLDFACDF |
| | | WVLVVVGGVLACYSLLVTVAFIIFWVRSKRSRLLHSD |
| | | YMNMTPRRPGPTRKHYQPYAPPRDFAAYRSLRVKFS |
| | | RSADAPAYQQGQNQLYNELNLGRREEYDVLDKRRGR |
| | | DPEMGGKPRRKNPQEGLYNELQKDKMAEAYSEIGMK |
| | | GERRRGKGHDGLYQGLSTATKDTYDALHMQALPPR |
| 49 | MycT-Affy7 CAR- | GSEQKLISEEDLGSQVQLQQSGPELVRPGASVKMSCR |
| | SSTR2 (control) | ASGYTFTSYWLHWVKQRPGQGLEWIGMIDPSNSDTR |
| | | FNPNFKDKATLNVDRSSNTAYMLLSSLTSADSAVYYC |
| | | ATYGSYVSPLDYWGQGTSVTVSSGGGGSGGGGSGGGG |
| | | SDIMMSQSPSSLTVSVGEKVTVSCKSSQSLLYTSSQKN |
| | | YLAWYQQKPGQSPKLLIYWASTRESGVPDRFTGSGSG |
| | | TDFTLTITSVKADDLAVYYCQQYYAYPWTFGGGTKL |
| | | EIKRASTTTPAPRPPTPAPTIASQPLSLRPEACRPAAGG |
| | | AVHTRGLDFACDFWVLVVVGGVLACYSLLVTVAFIIF |
| | | WVRSKRSRLLHSDYMNMTPRRPGPTRKHYQPYAPPR |
| | | DFAAYRSLRVKFSRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPRTSGSGATNFSLLKQAGDVEENPGPS |
| | | RMDMADEPLNGSHTWLSIPFDLNGSVVSTNTSNQTEP |
| | | YYDLTSNAVLTFIYFVVCIIGLCGNTLVIYVILRYAKM |
| | | KTITNIYILNLAIADELFMLGLPFLAMQVALVHWPFGK |
| | | AICRVVMTVDGINQFTSIFCLTVMSIDRYLAVVHPIKS |
| | | AKWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 50 | MycT-Affy7-Y6A | GSEQKLISEEDLGSQVQLQQSGPELVRPGASVKMSCR |
| | CAR-SSTR2 | ASGYTFTSYWLHWVKQRPGQGLEWIGMIDPSNSDTR |
| | | FNPNFKDKATLNVDRSSNTAYMLLSSLTSADSAVYYC |
| | | ATAGSYVSPLDYWGQGTSVTVSSGGGGSGGGGSGGGG |
| | | SDIMMSQSPSSLTVSVGEKVTVSCKSSQSLLYTSSQKN |
| | | YLAWYQQKPGQSPKLLIYWASTRESGVPDRFTGSGSG |
| | | TDFTLTITSVKADDLAVYYCQQYYAYPWTFGGGTKL |
| | | EIKRASTTTPAPRPPTPAPTIASQPLSLRPEACRPAAGG |
| | | AVHTRGLDFACDFWVLVVVGGVLACYSLLVTVAFIIF |
| | | WVRSKRSRLLHSDYMNMTPRRPGPTRKHYQPYAPPR |
| | | DFAAYRSLRVKFSRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPRTSGSGATNFSLLKQAGDVEENPGPS |
| | | RMDMADEPLNGSHTWLSIPFDLNGSVVSTNTSNQTEP |
| | | YYDLTSNAVLTFIYFVVCIIGLCGNTLVIYVILRYAKM |
| | | KTITNIYILNLAIADELFMLGLPFLAMQVALVHWPFGK |
| | | AICRVVMTVDGINQFTSIFCLTVMSIDRYLAVVHPIKS |
| | | AKWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 51 | MycT-Affy7-L9A | GSEQKLISEEDLGSQVQLQQSGPELVRPGASVKMSCR |
| | CAR-SSTR2 | ASGYTFTSYWLHWVKQRPGQGLEWIGMIDPSNSDTR |
| | | FNPNFKDKATLNVDRSSNTAYMLLSSLTSADSAVYYC |
| | | ATYGSYVSPADYWGQGTSVTVSSGGGGSGGGGSGGGG |
| | | SDIMMSQSPSSLTVSVGEKVTVSCKSSQSLLYTSSQKN |
| | | YLAWYQQKPGQSPKLLIYWASTRESGVPDRFTGSGSG |
| | | TDFTLTITSVKADDLAVYYCQQYYAYPWTFGGGTKL |
| | | EIKRASTTTPAPRPPTPAPTIASQPLSLRPEACRPAAGG |
| | | AVHTRGLDFACDFWVLVVVGGVLACYSLLVTVAFIIF |
| | | WVRSKRSRLLHSDYMNMTPRRPGPTRKHYQPYAPPR |
| | | DFAAYRSLRVKFSRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPRTSGSGATNFSLLKQAGDVEENPGPS |
| | | RMDMADEPLNGSHTWLSIPFDLNGSVVSTNTSNQTEP |
| | | YYDLTSNAVLTFIYFVVCIIGLCGNTLVIYVILRYAKM |
| | | KTITNIYILNLAIADELFMLGLPFLAMQVALVHWPFGK |
| | | AICRVVMTVDGINQFTSIFCLTVMSIDRYLAVVHPIKS |
| | | AKWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 52 | MycT-Affy7-Y6V | GSEQKLISEEDLGSQVQLQQSGPELVRPGASVKMSCR |
| | CAR-SSTR2 | ASGYTFTSYWLHWVKQRPGQGLEWIGMIDPSNSDTR |
| | | FNPNFKDKATLNVDRSSNTAYMLLSSLTSADSAVYYC |
| | | ATVGSYVSPLDYWGQGTSVTVSSGGGGSGGGGSGGGG |
| | | SDIMMSQSPSSLTVSVGEKVTVSCKSSQSLLYTSSQKN |
| | | YLAWYQQKPGQSPKLLIYWASTRESGVPDRFTGSGSG |
| | | TDFTLTITSVKADDLAVYYCQQYYAYPWTFGGGTKL |
| | | EIKRASTTTPAPRPPTPAPTIASQPLSLRPEACRPAAGG |
| | | AVHTRGLDFACDFWVLVVVGGVLACYSLLVTVAFIIF |
| | | WVRSKRSRLLHSDYMNMTPRRPGPTRKHYQPYAPPR |
| | | DFAAYRSLRVKFSRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPRTSGSGATNFSLLKQAGDVEENPGPS |
| | | RMDMADEPLNGSHTWLSIPFDLNGSVVSTNTSNQTEP |
| | | YYDLTSNAVLTFIYFVVCIIGLCGNTLVIYVILRYAKM |
| | | KTITNIYILNLAIADELFMLGLPFLAMQVALVHWPFGK |
| | | AICRVVMTVDGINQFTSIFCLTVMSIDRYLAVVHPIKS |
| | | AKWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 53 | MycT-Affy7-Y6L | GSEQKLISEEDLGSQVQLQQSGPELVRPGASVKMSCR |
| | CAR-SSTR2 | ASGYTFTSYWLHWVKQRPGQGLEWIGMIDPSNSDTR |
| | | FNPNFKDKATLNVDRSSNTAYMLLSSLTSADSAVYYC |
| | | ATLGSYVSPLDYWGQGTSVTVSSGGGGSGGGGSGGGG |
| | | SDIMMSQSPSSLTVSVGEKVTVSCKSSQSLLYTSSQKN |
| | | YLAWYQQKPGQSPKLLIYWASTRESGVPDRFTGSGSG |
| | | TDFTLTITSVKADDLAVYYCQQYYAYPWTFGGGTKL |
| | | EIKRASTTTPAPRPPTPAPTIASQPLSLRPEACRPAAGG |
| | | AVHTRGLDFACDFWVLVVVGGVLACYSLLVTVAFIIF |
| | | WVRSKRSRLLHSDYMNMTPRRPGPTRKHYQPYAPPR |
| | | DFAAYRSLRVKFSRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPRTSGSGATNFSLLKQAGDVEENPGPS |
| | | RMDMADEPLNGSHTWLSIPFDLNGSVVSTNTSNQTEP |
| | | YYDLTSNAVLTFIYFVVCIIGLCGNTLVIYVILRYAKM |
| | | KTITNIYILNLAIADELFMLGLPFLAMQVALVHWPFGK |
| | | AICRVVMTVDGINQFTSIFCLTVMSIDRYLAVVHPIKS |
| | | AKWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 54 | MycT-Affy7-Y6I | GSEQKLISEEDLGSQVQLQQSGPELVRPGASVKMSCR |
| | CAR-SSTR2 | ASGYTFTSYWLHWVKQRPGQGLEWIGMIDPSNSDTR |
| | | FNPNFKDKATLNVDRSSNTAYMLLSSLTSADSAVYYC |
| | | ATIGSYVSPLDYWGQGTSVTVSSGGGGSGGGGSGGGG |
| | | SDIMMSQSPSSLTVSVGEKVTVSCKSSQSLLYTSSQKN |
| | | YLAWYQQKPGQSPKLLIYWASTRESGVPDRFTGSGSG |
| | | TDFTLTITSVKADDLAVYYCQQYYAYPWTFGGGTKL |
| | | EIKRASTTTPAPRPPTPAPTIASQPLSLRPEACRPAAGG |
| | | AVHTRGLDFACDFWVLVVVGGVLACYSLLVTVAFIIF |
| | | WVRSKRSRLLHSDYMNMTPRRPGPTRKHYQPYAPPR |
| | | DFAAYRSLRVKFSRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPRTSGSGATNFSLLKQAGDVEENPGPS |
| | | RMDMADEPLNGSHTWLSIPFDLNGSVVSTNTSNQTEP |
| | | YYDLTSNAVLTFIYFVVCIIGLCGNTLVIYVILRYAKM |
| | | KTITNIYILNLAIADELFMLGLPFLAMQVALVHWPFGK |
| | | AICRVVMTVDGINQFTSIFCLTVMSIDRYLAVVHPIKS |
| | | AKWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 55 | MycT-Affy7-Y6M | GSEQKLISEEDLGSQVQLQQSGPELVRPGASVKMSCR |
| | CAR-SSTR2 | ASGYTFTSYWLHWVKQRPGQGLEWIGMIDPSNSDTR |
| | | FNPNFKDKATLNVDRSSNTAYMLLSSLTSADSAVYYC |
| | | ATMGSYVSPLDYWGQGTSVTVSSGGGGSGGGGSGGG |
| | | GSDIMMSQSPSSLTVSVGEKVTVSCKSSQSLLYTSSQK |
| | | NYLAWYQQKPGQSPKLLIYWASTRESGVPDRFTGSGS |
| | | GTDFTLTITSVKADDLAVYYCQQYYAYPWTFGGGTK |
| | | LEIKRASTTTPAPRPPTPAPTIASQPLSLRPEACRPAAG |
| | | GAVHTRGLDFACDFWVLVVVGGVLACYSLLVTVAFII |
| | | FWVRSKRSRLLHSDYMNMTPRRPGPTRKHYQPYAPP |
| | | RDFAAYRSLRVKFSRSADAPAYQQGQNQLYNELNLG |
| | | RREEYDVLDKRRGRDPEMGGKPRRKNPQEGLYNELQ |
| | | KDKMAEAYSEIGMKGERRRGKGHDGLYQGLSTATK |
| | | DTYDALHMQALPPRTSGSGATNFSLLKQAGDVEENP |
| | | GPSRMDMADEPLNGSHTWLSIPFDLNGSVVSTNTSNQ |
| | | TEPYYDLTSNAVLTFIYFVVCIIGLCGNTLVIYVILRYA |
| | | KMKTITNIYILNLAIADELFMLGLPFLAMQVALVHWP |
| | | FGKAICRVVMTVDGINQFTSIFCLTVMSIDRYLAVVHP |
| | | IKSAKWRRPRTAKMITMAVWGVSLLVILPIMIYAGLR |
| | | SNQWGRSSCTINWPGESGAWYTGFIIYTFILGFLVPLTI |
| | | ICLCYLFIIIKVKSSGIRVGSSKRKKSEKKVTRMVSIVV |
| | | AVFIFCWLPFYIFNVSSVSMAISPTPALKGMFDFVVVL |
| | | TYANSCANPILYAFLSDNFKKSFQNVLCLVKVSGTDD |
| | | GERSDSKQDKSRLNETTETQRTLLNGDLQTSI |
| 56 | Affy7 CAR (control) | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCTACGGCTCCTACGTGAGCCCTTTGGATT |
| | | ATTGGGGCCAGGGCACCTCCGTCACCGTGTCCTCCgg |
| | | tggaggcggttcaggcggaggtggctctggcggtggcggatcgGACATCAT |
| | | GATGTCTCAGAGTCCCTCTTCTCTGACCGTGTCCGT |
| | | GGGCGAGAAGGTGACCGTGTCATGCAAGTCCTCAC |
| | | AGAGCCTTCTGTACACCAGCTCCCAGAAGAACTAC |
| | | CTGGCGTGGTACCAGCAGAAGCCCGGACAGAGCCC |
| | | CAAGCTGCTGATCTATTGGGCTTCCACCCGCGAGAG |
| | | CGGCGTCCCCGACCGCTTCACCGGCTCCGGCTCCGG |
| | | GACCGACTTCACCCTGACCATCACCTCCGTGAAGGC |
| | | CGATGACCTGGCCGTGTACTACTGTCAACAGTATTA |
| | | CGCCTACCCGTGGACCTTCGGAGGCGGCACCAAGC |
| | | TGGAGATCAAGCGCgctagcacgaccactccggcgccgcgcccaccg |
| | | actccggccccaactatcgcgagccagcccctgtcgctgaggccggaagcatgccg |
| | | ccctgccgccggaggtgctgtgcatacccggggattggacttcgcatgcgacTTT |
| | | TGGGTGCTGGTGGTGGTTGGTGGAGTCCTGGCTTGC |
| | | TATAGCTTGCTAGTAACAGTGGCCTTTATTATTTTCT |
| | | GGGTGAGGAGTAAGAGGAGCAGGCTCCTGCACAGT |
| | | GACTACATGAACATGACTCCCCGCCGCCCCGGGCC |
| | | CACCCGCAAGCATTACCAGCCCTATGCCCCACCACG |
| | | CGACTTCGCAGCCTATCGCTCCctgagagtgaagttcagcagga |
| | | gcgcagacgcccccgcgtaccagcagggccagaaccagctctataacgagctcaa |
| | | tctaggacgaagagaggagtacgatgttttggacaagagacgtggccgggaccctg |
| | | agatggggggaaagccgagaaggaagaaccctcaggaaggcctgtacaatgaact |
| | | gcagaaagataagatggcggaggcctacagtgagattgggatgaaaggcgagcgc |
| | | cggaggggcaaggggcacgatggcctttaccagggtctcagtacagccaccaagg |
| | | acacctacgacgcccttcacatgcaggccctgccccctcgc |
| 57 | Affy7-Y6A CAR | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCGCGGGCTCCTACGTGAGCCCTTTGGAT |
| | | TATTGGGGCCAGGGCACCTCCGTCACCGTGTCCTCC |
| | | ggtggaggcggttcaggcggaggtggctctggcggtggcggatcgGACATC |
| | | ATGATGTCTCAGAGTCCCTCTTCTCTGACCGTGTCC |
| | | GTGGGCGAGAAGGTGACCGTGTCATGCAAGTCCTC |
| | | ACAGAGCCTTCTGTACACCAGCTCCCAGAAGAACT |
| | | ACCTGGCGTGGTACCAGCAGAAGCCCGGACAGAGC |
| | | CCCAAGCTGCTGATCTATTGGGCTTCCACCCGCGAG |
| | | AGCGGCGTCCCCGACCGCTTCACCGGCTCCGGCTCC |
| | | GGGACCGACTTCACCCTGACCATCACCTCCGTGAAG |
| | | GCCGATGACCTGGCCGTGTACTACTGTCAACAGTAT |
| | | TACGCCTACCCGTGGACCTTCGGAGGCGGCACCAA |
| | | GCTGGAGATCAAGCGCgctagcacgaccactccggcgccgcgccca |
| | | ccgactccggccccaactatcgcgagccagcccctgtcgctgaggccggaagcatg |
| | | ccgccctgccgccggaggtgctgtgcatacccggggattggacttcgcatgcgacT |
| | | TTTGGGTGCTGGTGGTGGTTGGTGGAGTCCTGGCTT |
| | | GCTATAGCTTGCTAGTAACAGTGGCCTTTATTATTT |
| | | TCTGGGTGAGGAGTAAGAGGAGCAGGCTCCTGCAC |
| | | AGTGACTACATGAACATGACTCCCCGCCGCCCCGG |
| | | GCCCACCCGCAAGCATTACCAGCCCTATGCCCCACC |
| | | ACGCGACTTCGCAGCCTATCGCTCCctgagagtgaagttcagc |
| | | aggagcgcagacgcccccgcgtaccagcagggccagaaccagctctataacgag |
| | | ctcaatctaggacgaagagaggagtacgatgttttggacaagagacgtggccggga |
| | | ccctgagatggggggaaagccgagaaggaagaaccctcaggaaggcctgtacaat |
| | | gaactgcagaaagataagatggcggaggcctacagtgagattgggatgaaaggcg |
| | | agcgccggaggggcaaggggcacgatggcctttaccagggtctcagtacagccac |
| | | caaggacacctacgacgcccttcacatgcaggccctgccccctcgc |
| 58 | Affy7-L9A CAR | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCTACGGCTCCTACGTGAGCCCTGCGGAT |
| | | TATTGGGGCCAGGGCACCTCCGTCACCGTGTCCTCC |
| | | ggtggaggcggttcaggcggaggtggctctggcggtggcggatcgGACATC |
| | | ATGATGTCTCAGAGTCCCTCTTCTCTGACCGTGTCC |
| | | GTGGGCGAGAAGGTGACCGTGTCATGCAAGTCCTC |
| | | ACAGAGCCTTCTGTACACCAGCTCCCAGAAGAACT |
| | | ACCTGGCGTGGTACCAGCAGAAGCCCGGACAGAGC |
| | | CCCAAGCTGCTGATCTATTGGGCTTCCACCCGCGAG |
| | | AGCGGCGTCCCCGACCGCTTCACCGGCTCCGGCTCC |
| | | GGGACCGACTTCACCCTGACCATCACCTCCGTGAAG |
| | | GCCGATGACCTGGCCGTGTACTACTGTCAACAGTAT |
| | | TACGCCTACCCGTGGACCTTCGGAGGCGGCACCAA |
| | | GCTGGAGATCAAGCGCgctagcacgaccactccggcgccgcgccca |
| | | ccgactccggccccaactatcgcgagccagcccctgtcgctgaggccggaagcatg |
| | | ccgccctgccgccggaggtgctgtgcatacccggggattggacttcgcatgcgacT |
| | | TTTGGGTGCTGGTGGTGGTTGGTGGAGTCCTGGCTT |
| | | GCTATAGCTTGCTAGTAACAGTGGCCTTTATTATTT |
| | | TCTGGGTGAGGAGTAAGAGGAGCAGGCTCCTGCAC |
| | | AGTGACTACATGAACATGACTCCCCGCCGCCCCGG |
| | | GCCCACCCGCAAGCATTACCAGCCCTATGCCCCACC |
| | | ACGCGACTTCGCAGCCTATCGCTCCctgagagtgaagttcagc |
| | | aggagcgcagacgcccccgcgtaccagcagggccagaaccagctctataacgag |
| | | ctcaatctaggacgaagagaggagtacgatgttttggacaagagacgtggccggga |
| | | ccctgagatggggggaaagccgagaaggaagaaccctcaggaaggcctgtacaat |
| | | gaactgcagaaagataagatggcggaggcctacagtgagattgggatgaaaggcg |
| | | agcgccggaggggcaaggggcacgatggcctttaccagggtctcagtacagccac |
| | | caaggacacctacgacgcccttcacatgcaggccctgccccctcgc |
| 59 | Affy7-Y6V CAR | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCGTCGGCTCCTACGTGAGCCCTTTGGAT |
| | | TATTGGGGCCAGGGCACCTCCGTCACCGTGTCCTCC |
| | | ggtggaggcggttcaggcggaggtggctctggcggtggcggatcgGACATC |
| | | ATGATGTCTCAGAGTCCCTCTTCTCTGACCGTGTCC |
| | | GTGGGCGAGAAGGTGACCGTGTCATGCAAGTCCTC |
| | | ACAGAGCCTTCTGTACACCAGCTCCCAGAAGAACT |
| | | ACCTGGCGTGGTACCAGCAGAAGCCCGGACAGAGC |
| | | CCCAAGCTGCTGATCTATTGGGCTTCCACCCGCGAG |
| | | AGCGGCGTCCCCGACCGCTTCACCGGCTCCGGCTCC |
| | | GGGACCGACTTCACCCTGACCATCACCTCCGTGAAG |
| | | GCCGATGACCTGGCCGTGTACTACTGTCAACAGTAT |
| | | TACGCCTACCCGTGGACCTTCGGAGGCGGCACCAA |
| | | GCTGGAGATCAAGCGCgctagcacgaccactccggcgccgcgccca |
| | | ccgactccggccccaactatcgcgagccagcccctgtcgctgaggccggaagcatg |
| | | ccgccctgccgccggaggtgctgtgcatacccggggattggacttcgcatgcgacT |
| | | TTTGGGTGCTGGTGGTGGTTGGTGGAGTCCTGGCTT |
| | | GCTATAGCTTGCTAGTAACAGTGGCCTTTATTATTT |
| | | TCTGGGTGAGGAGTAAGAGGAGCAGGCTCCTGCAC |
| | | AGTGACTACATGAACATGACTCCCCGCCGCCCCGG |
| | | GCCCACCCGCAAGCATTACCAGCCCTATGCCCCACC |
| | | ACGCGACTTCGCAGCCTATCGCTCCctgagagtgaagttcagc |
| | | aggagcgcagacgcccccgcgtaccagcagggccagaaccagctctataacgag |
| | | ctcaatctaggacgaagagaggagtacgatgttttggacaagagacgtggccggga |
| | | ccctgagatggggggaaagccgagaaggaagaaccctcaggaaggcctgtacaat |
| | | gaactgcagaaagataagatggcggaggcctacagtgagattgggatgaaaggcg |
| | | agcgccggaggggcaaggggcacgatggcctttaccagggtctcagtacagccac |
| | | caaggacacctacgacgcccttcacatgcaggccctgccccctcgc |
| 60 | Affy7-Y6L CAR | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCCTCGGCTCCTACGTGAGCCCTTTGGAT |
| | | TATTGGGGCCAGGGCACCTCCGTCACCGTGTCCTCC |
| | | ggtggaggcggttcaggcggaggtggctctggcggtggcggatcgGACATC |
| | | ATGATGTCTCAGAGTCCCTCTTCTCTGACCGTGTCC |
| | | GTGGGCGAGAAGGTGACCGTGTCATGCAAGTCCTC |
| | | ACAGAGCCTTCTGTACACCAGCTCCCAGAAGAACT |
| | | ACCTGGCGTGGTACCAGCAGAAGCCCGGACAGAGC |
| | | CCCAAGCTGCTGATCTATTGGGCTTCCACCCGCGAG |
| | | AGCGGCGTCCCCGACCGCTTCACCGGCTCCGGCTCC |
| | | GGGACCGACTTCACCCTGACCATCACCTCCGTGAAG |
| | | GCCGATGACCTGGCCGTGTACTACTGTCAACAGTAT |
| | | TACGCCTACCCGTGGACCTTCGGAGGCGGCACCAA |
| | | GCTGGAGATCAAGCGCgctagcacgaccactccggcgccgcgccca |
| | | ccgactccggccccaactatcgcgagccagcccctgtcgctgaggccggaagcatg |
| | | ccgccctgccgccggaggtgctgtgcatacccggggattggacttcgcatgcgacT |
| | | TTTGGGTGCTGGTGGTGGTTGGTGGAGTCCTGGCTT |
| | | GCTATAGCTTGCTAGTAACAGTGGCCTTTATTATTT |
| | | TCTGGGTGAGGAGTAAGAGGAGCAGGCTCCTGCAC |
| | | AGTGACTACATGAACATGACTCCCCGCCGCCCCGG |
| | | GCCCACCCGCAAGCATTACCAGCCCTATGCCCCACC |
| | | ACGCGACTTCGCAGCCTATCGCTCCctgagagtgaagttcagc |
| | | aggagcgcagacgcccccgcgtaccagcagggccagaaccagctctataacgag |
| | | ctcaatctaggacgaagagaggagtacgatgttttggacaagagacgtggccggga |
| | | ccctgagatggggggaaagccgagaaggaagaaccctcaggaaggcctgtacaat |
| | | gaactgcagaaagataagatggcggaggcctacagtgagattgggatgaaaggcg |
| | | agcgccggaggggcaaggggcacgatggcctttaccagggtctcagtacagccac |
| | | caaggacacctacgacgcccttcacatgcaggccctgccccctcgc |
| 61 | Affy7-Y6I CAR | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCATCGGCTCCTACGTGAGCCCTTTGGAT |
| | | TATTGGGGCCAGGGCACCTCCGTCACCGTGTCCTCC |
| | | ggtggaggcggttcaggcggaggtggctctggcggtggcggatcgGACATC |
| | | ATGATGTCTCAGAGTCCCTCTTCTCTGACCGTGTCC |
| | | GTGGGCGAGAAGGTGACCGTGTCATGCAAGTCCTC |
| | | ACAGAGCCTTCTGTACACCAGCTCCCAGAAGAACT |
| | | ACCTGGCGTGGTACCAGCAGAAGCCCGGACAGAGC |
| | | CCCAAGCTGCTGATCTATTGGGCTTCCACCCGCGAG |
| | | AGCGGCGTCCCCGACCGCTTCACCGGCTCCGGCTCC |
| | | GGGACCGACTTCACCCTGACCATCACCTCCGTGAAG |
| | | GCCGATGACCTGGCCGTGTACTACTGTCAACAGTAT |
| | | TACGCCTACCCGTGGACCTTCGGAGGCGGCACCAA |
| | | GCTGGAGATCAAGCGCgctagcacgaccactccggcgccgcgccca |
| | | ccgactccggccccaactatcgcgagccagcccctgtcgctgaggccggaagcatg |
| | | ccgccctgccgccggaggtgctgtgcatacccggggattggacttcgcatgcgacT |
| | | TTTGGGTGCTGGTGGTGGTTGGTGGAGTCCTGGCTT |
| | | GCTATAGCTTGCTAGTAACAGTGGCCTTTATTATTT |
| | | TCTGGGTGAGGAGTAAGAGGAGCAGGCTCCTGCAC |
| | | AGTGACTACATGAACATGACTCCCCGCCGCCCCGG |
| | | GCCCACCCGCAAGCATTACCAGCCCTATGCCCCACC |
| | | ACGCGACTTCGCAGCCTATCGCTCCctgagagtgaagttcagc |
| | | aggagcgcagacgcccccgcgtaccagcagggccagaaccagctctataacgag |
| | | ctcaatctaggacgaagagaggagtacgatgttttggacaagagacgtggccggga |
| | | ccctgagatggggggaaagccgagaaggaagaaccctcaggaaggcctgtacaat |
| | | gaactgcagaaagataagatggcggaggcctacagtgagattgggatgaaaggcg |
| | | agcgccggaggggcaaggggcacgatggcctttaccagggtctcagtacagccac |
| | | caaggacacctacgacgcccttcacatgcaggccctgccccctcgc |
| 62 | Affy7-Y6M CAR | CAGGTGCAGCTCCAGCAGAGCGGCCCTGAGCTGGT |
| | | GCGCCCCGGCGCTTCCGTGAAGATGTCATGCCGAG |
| | | CTTCTGGCTACACGTTCACCAGCTATTGGCTACACT |
| | | GGGTCAAGCAGAGGCCCGGACAGGGTCTGGAGTGG |
| | | ATCGGTATGATTGACCCGTCAAATAGTGACACCCGC |
| | | TTCAACCCCAACTTCAAGGACAAGGCCACTCTCAAC |
| | | GTGGACCGCTCGAGCAACACGGCCTACATGCTGCT |
| | | GTCCTCTCTGACCTCGGCGGACAGCGCCGTGTACTA |
| | | CTGTGCCACCATGGGCTCCTACGTGAGCCCTTTGGAT |
| | | TATTGGGGCCAGGGCACCTCCGTCACCGTGTCCTCC |
| | | ggtggaggcggttcaggcggaggtggctctggcggtggcggatcgGACATC |
| | | ATGATGTCTCAGAGTCCCTCTTCTCTGACCGTGTCC |
| | | GTGGGCGAGAAGGTGACCGTGTCATGCAAGTCCTC |
| | | ACAGAGCCTTCTGTACACCAGCTCCCAGAAGAACT |
| | | ACCTGGCGTGGTACCAGCAGAAGCCCGGACAGAGC |
| | | CCCAAGCTGCTGATCTATTGGGCTTCCACCCGCGAG |
| | | AGCGGCGTCCCCGACCGCTTCACCGGCTCCGGCTCC |
| | | GGGACCGACTTCACCCTGACCATCACCTCCGTGAAG |
| | | GCCGATGACCTGGCCGTGTACTACTGTCAACAGTAT |
| | | TACGCCTACCCGTGGACCTTCGGAGGCGGCACCAA |
| | | GCTGGAGATCAAGCGCgctagcacgaccactccggcgccgcgccca |
| | | ccgactccggccccaactatcgcgagccagcccctgtcgctgaggccggaagcatg |
| | | ccgccctgccgccggaggtgctgtgcatacccggggattggacttcgcatgcgacT |
| | | TTTGGGTGCTGGTGGTGGTTGGTGGAGTCCTGGCTT |
| | | GCTATAGCTTGCTAGTAACAGTGGCCTTTATTATTT |
| | | TCTGGGTGAGGAGTAAGAGGAGCAGGCTCCTGCAC |
| | | AGTGACTACATGAACATGACTCCCCGCCGCCCCGG |
| | | GCCCACCCGCAAGCATTACCAGCCCTATGCCCCACC |
| | | ACGCGACTTCGCAGCCTATCGCTCCctgagagtgaagttcagc |
| | | aggagcgcagacgcccccgcgtaccagcagggccagaaccagctctataacgag |
| | | ctcaatctaggacgaagagaggagtacgatgttttggacaagagacgtggccggga |
| | | ccctgagatggggggaaagccgagaaggaagaaccctcaggaaggcctgtacaat |
| | | gaactgcagaaagataagatggcggaggcctacagtgagattgggatgaaaggcg |
| | | agcgccggaggggcaaggggcacgatggcctttaccagggtctcagtacagccac |
| | | caaggacacctacgacgcccttcacatgcaggccctgccccctcgc |
| 63 | EF1α-MycT-Affy7 | ggctccggtgcccgtcagtgggcagagcgcacatcgcccacagtccccgagaagtt |
| | CAR-SSTR2 | ggggggaggggtcggcaattgaaccggtgcctagagaaggtggcgcggggtaaa |
| | (control) | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtgggtgggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagtt |
| | | tttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCCAGCAGAGCGGCCCTGAGCTGGTGCGCCCCGG |
| | | CGCTTCCGTGAAGATGTCATGCCGAGCTTCTGGCTA |
| | | CACGTTCACCAGCTATTGGCTACACTGGGTCAAGCA |
| | | GAGGCCCGGACAGGGTCTGGAGTGGATCGGTATGA |
| | | TTGACCCGTCAAATAGTGACACCCGCTTCAACCCCA |
| | | ACTTCAAGGACAAGGCCACTCTCAACGTGGACCGC |
| | | TCGAGCAACACGGCCTACATGCTGCTGTCCTCTCTG |
| | | ACCTCGGCGGACAGCGCCGTGTACTACTGTGCCACC |
| | | TACGGCTCCTACGTGAGCCCTTTGGATTATTGGGGCCA |
| | | GGGCACCTCCGTCACCGTGTCCTCCggtggaggcggttcagg |
| | | cggaggtggctctggcggtggcggatcgGACATCATGATGTCTCA |
| | | GAGTCCCTCTTCTCTGACCGTGTCCGTGGGCGAGAA |
| | | GGTGACCGTGTCATGCAAGTCCTCACAGAGCCTTCT |
| | | GTACACCAGCTCCCAGAAGAACTACCTGGCGTGGT |
| | | ACCAGCAGAAGCCCGGACAGAGCCCCAAGCTGCTG |
| | | ATCTATTGGGCTTCCACCCGCGAGAGCGGCGTCCCC |
| | | GACCGCTTCACCGGCTCCGGCTCCGGGACCGACTTC |
| | | ACCCTGACCATCACCTCCGTGAAGGCCGATGACCTG |
| | | GCCGTGTACTACTGTCAACAGTATTACGCCTACCCG |
| | | TGGACCTTCGGAGGCGGCACCAAGCTGGAGATCAA |
| | | GCGCgctagcacgaccactccggcgccgcgcccaccgactccggccccaacta |
| | | tcgcgagccagcccctgtcgctgaggccggaagcatgccgccctgccgccggagg |
| | | tgctgtgcatacccggggattggacttcgcatgcgacTTTTGGGTGCTGG |
| | | TGGTGGTTGGTGGAGTCCTGGCTTGCTATAGCTTGC |
| | | TAGTAACAGTGGCCTTTATTATTTTCTGGGTGAGGA |
| | | GTAAGAGGAGCAGGCTCCTGCACAGTGACTACATG |
| | | AACATGACTCCCCGCCGCCCCGGGCCCACCCGCAA |
| | | GCATTACCAGCCCTATGCCCCACCACGCGACTTCGC |
| | | AGCCTATCGCTCCctgagagtgaagttcagcaggagcgcagacgcccc |
| | | cgcgtaccagcagggccagaaccagctctataacgagctcaatctaggacgaagag |
| | | aggagtacgatgttttggacaagagacgtggccgggaccctgagatggggggaaa |
| | | gccgagaaggaagaaccctcaggaaggcctgtacaatgaactgcagaaagataag |
| | | atggcggaggcctacagtgagattgggatgaaaggcgagcgccggaggggcaag |
| | | gggcacgatggcctttaccagggtctcagtacagccaccaaggacacctacgacgc |
| | | ccttcacatgcaggccctgccccctcgcactagtggaagcggagctactaacttcag |
| | | cctgctgaagcaggctggagacgtggaggagaaccctggaccttctagaatggaca |
| | | tggcggatgagccactcaatggaagccacacatggctatccattccatttgacctcaat |
| | | ggctctgtggtgtcaaccaacacctcaaaccagacagagccgtactatgacctgaca |
| | | agcaatgcagtcctcacattcatctattttgtggtctgcatcattgggttgtgtggcaaca |
| | | cacttgtcatttatgtcatcctccgctatgccaagatgaagaccatcaccaacatttacat |
| | | cctcaacctggccatcgcagatgagctcttcatgctgggtctgcctttcttggctatgca |
| | | ggtggctctggtccactggccctttggcaaggccatttgccgggtggtcatgactgtg |
| | | gatggcatcaatcagttcaccagcatcttctgcctgacagtcatgagcatcgaccgata |
| | | cctggctgtggtccaccccatcaagtcggccaagtggaggagaccccggacggcc |
| | | aagatgatcaccatggctgtgtggggagtctctctgctggtcatcttgcccatcatgata |
| | | tatgctgggctccggagcaaccagtgggggagaagcagctgcaccatcaactggcc |
| | | aggtgaatctggggcttggtacacagggttcatcatctacactttcattctggggttcct |
| | | ggtacccctcaccatcatctgtctttgctacctgttcattatcatcaaggtgaagtcctctg |
| | | gaatccgagtgggctcctctaagaggaagaagtctgagaagaaggtcacccgaatg |
| | | gtgtccatcgtggtggctgtcttcatcttctgctggcttcccttctacatattcaacgtttc |
| | | ttccgtctccatggccatcagccccaccccagcccttaaaggcatgtttgactttgtggtg |
| | | gtcctcacctatgctaacagctgtgccaaccctatcctatatgccttcttgtctgacaact |
| | | tcaagaagagcttccagaatgtcctctgcttggtcaaggtgagcggcacagatgatgg |
| | | ggagcggagtgacagtaagcaggacaaatcccggctgaatgagaccacggagac |
| | | ccagaggaccctcctcaatggagacctccaaaccagtatctaa |
| 64 | EF1α-MycT-Affy7- | ggctccggtgcccgtcagtgggcagagcgcacatcgcccacagtccccgagaagtt |
| | Y6A CAR-SSTR2 | ggggggaggggtcggcaattgaaccggtgcctagagaaggtggcgcggggtaaa |
| | | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtgggtgggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagtt |
| | | tttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCCAGCAGAGCGGCCCTGAGCTGGTGCGCCCCGG |
| | | CGCTTCCGTGAAGATGTCATGCCGAGCTTCTGGCTA |
| | | CACGTTCACCAGCTATTGGCTACACTGGGTCAAGCA |
| | | GAGGCCCGGACAGGGTCTGGAGTGGATCGGTATGA |
| | | TTGACCCGTCAAATAGTGACACCCGCTTCAACCCCA |
| | | ACTTCAAGGACAAGGCCACTCTCAACGTGGACCGC |
| | | TCGAGCAACACGGCCTACATGCTGCTGTCCTCTCTG |
| | | ACCTCGGCGGACAGCGCCGTGTACTACTGTGCCACC |
| | | GCGGGCTCCTACGTGAGCCCTTTGGATTATTGGGGCC |
| | | AGGGCACCTCCGTCACCGTGTCCTCCggtggaggcggttca |
| | | ggcggaggtggctctggcggtggcggatcgGACATCATGATGTCTC |
| | | AGAGTCCCTCTTCTCTGACCGTGTCCGTGGGCGAGA |
| | | AGGTGACCGTGTCATGCAAGTCCTCACAGAGCCTTC |
| | | TGTACACCAGCTCCCAGAAGAACTACCTGGCGTGG |
| | | TACCAGCAGAAGCCCGGACAGAGCCCCAAGCTGCT |
| | | GATCTATTGGGCTTCCACCCGCGAGAGCGGCGTCCC |
| | | CGACCGCTTCACCGGCTCCGGCTCCGGGACCGACTT |
| | | CACCCTGACCATCACCTCCGTGAAGGCCGATGACCT |
| | | GGCCGTGTACTACTGTCAACAGTATTACGCCTACCC |
| | | GTGGACCTTCGGAGGCGGCACCAAGCTGGAGATCA |
| | | AGCGCgctagcacgaccactccggcgccgcgcccaccgactccggccccaac |
| | | tatcgcgagccagcccctgtcgctgaggccggaagcatgccgccctgccgccgga |
| | | ggtgctgtgcatacccggggattggacttcgcatgcgacTTTTGGGTGCTG |
| | | GTGGTGGTTGGTGGAGTCCTGGCTTGCTATAGCTTG |
| | | CTAGTAACAGTGGCCTTTATTATTTTCTGGGTGAGG |
| | | AGTAAGAGGAGCAGGCTCCTGCACAGTGACTACAT |
| | | GAACATGACTCCCCGCCGCCCCGGGCCCACCCGCA |
| | | AGCATTACCAGCCCTATGCCCCACCACGCGACTTCG |
| | | CAGCCTATCGCTCCctgagagtgaagttcagcaggagcgcagacgccc |
| | | ccgcgtaccagcagggccagaaccagctctataacgagctcaatctaggacgaaga |
| | | gaggagtacgatgttttggacaagagacgtggccgggaccctgagatggggggaa |
| | | agccgagaaggaagaaccctcaggaaggcctgtacaatgaactgcagaaagataa |
| | | gatggcggaggcctacagtgagattgggatgaaaggcgagcgccggaggggcaa |
| | | ggggcacgatggcctttaccagggtctcagtacagccaccaaggacacctacgacg |
| | | cccttcacatgcaggccctgccccctcgcactagtggaagcggagctactaacttca |
| | | gcctgctgaagcaggctggagacgtggaggagaaccctggaccttctagaatggac |
| | | atggcggatgagccactcaatggaagccacacatggctatccattccatttgacctca |
| | | atggctctgtggtgtcaaccaacacctcaaaccagacagagccgtactatgacctgac |
| | | aagcaatgcagtcctcacattcatctattttgtggtctgcatcattgggttgtgtggcaac |
| | | acacttgtcatttatgtcatcctccgctatgccaagatgaagaccatcaccaacatttac |
| | | atcctcaacctggccatcgcagatgagctcttcatgctgggtctgcctttcttggctatg |
| | | caggtggctctggtccactggccctttggcaaggccatttgccgggtggtcatgactg |
| | | tggatggcatcaatcagttcaccagcatcttctgcctgacagtcatgagcatcgaccga |
| | | tacctggctgtggtccaccccatcaagtcggccaagtggaggagaccccggacggc |
| | | caagatgatcaccatggctgtgtggggagtctctctgctggtcatcttgcccatcatgat |
| | | atatgctgggctccggagcaaccagtgggggagaagcagctgcaccatcaactggc |
| | | caggtgaatctggggcttggtacacagggttcatcatctacactttcattctggggttcc |
| | | tggtacccctcaccatcatctgtctttgctacctgttcattatcatcaaggtgaagtcctct |
| | | ggaatccgagtgggctcctctaagaggaagaagtctgagaagaaggtcacccgaat |
| | | ggtgtccatcgtggtggctgtcttcatcttctgctggcttcccttctacatattcaacgttt |
| | | cttccgtctccatggccatcagccccaccccagcccttaaaggcatgtttgactttgtggt |
| | | ggtcctcacctatgctaacagctgtgccaaccctatcctatatgccttcttgtctgacaac |
| | | ttcaagaagagcttccagaatgtcctctgcttggtcaaggtgagcggcacagatgatg |
| | | gggagcggagtgacagtaagcaggacaaatcccggctgaatgagaccacggaga |
| | | cccagaggaccctcctcaatggagacctccaaaccagtatctaa |
| 65 | EF1α-MycT-Affy7- | ggctccggtgcccgtcagtgggcagagcgcacatcgcccacagtccccgagaagtt |
| | L9A CAR-SSTR2 | ggggggaggggtcggcaattgaaccggtgcctagagaaggtggcgcggggtaaa |
| | | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtgggtgggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagtt |
| | | tttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCCAGCAGAGCGGCCCTGAGCTGGTGCGCCCCGG |
| | | CGCTTCCGTGAAGATGTCATGCCGAGCTTCTGGCTA |
| | | CACGTTCACCAGCTATTGGCTACACTGGGTCAAGCA |
| | | GAGGCCCGGACAGGGTCTGGAGTGGATCGGTATGA |
| | | TTGACCCGTCAAATAGTGACACCCGCTTCAACCCCA |
| | | ACTTCAAGGACAAGGCCACTCTCAACGTGGACCGC |
| | | TCGAGCAACACGGCCTACATGCTGCTGTCCTCTCTG |
| | | ACCTCGGCGGACAGCGCCGTGTACTACTGTGCCACC |
| | | TACGGCTCCTACGTGAGCCCTGCGGATTATTGGGGCC |
| | | AGGGCACCTCCGTCACCGTGTCCTCCggtggaggcggttca |
| | | ggcggaggtggctctggcggtggcggatcgGACATCATGATGTCTC |
| | | AGAGTCCCTCTTCTCTGACCGTGTCCGTGGGCGAGA |
| | | AGGTGACCGTGTCATGCAAGTCCTCACAGAGCCTTC |
| | | TGTACACCAGCTCCCAGAAGAACTACCTGGCGTGG |
| | | TACCAGCAGAAGCCCGGACAGAGCCCCAAGCTGCT |
| | | GATCTATTGGGCTTCCACCCGCGAGAGCGGCGTCCC |
| | | CGACCGCTTCACCGGCTCCGGCTCCGGGACCGACTT |
| | | CACCCTGACCATCACCTCCGTGAAGGCCGATGACCT |
| | | GGCCGTGTACTACTGTCAACAGTATTACGCCTACCC |
| | | GTGGACCTTCGGAGGCGGCACCAAGCTGGAGATCA |
| | | AGCGCgctagcacgaccactccggcgccgcgcccaccgactccggccccaac |
| | | tatcgcgagccagcccctgtcgctgaggccggaagcatgccgccctgccgccgga |
| | | ggtgctgtgcatacccggggattggacttcgcatgcgacTTTTGGGTGCTG |
| | | GTGGTGGTTGGTGGAGTCCTGGCTTGCTATAGCTTG |
| | | CTAGTAACAGTGGCCTTTATTATTTTCTGGGTGAGG |
| | | AGTAAGAGGAGCAGGCTCCTGCACAGTGACTACAT |
| | | GAACATGACTCCCCGCCGCCCCGGGCCCACCCGCA |
| | | AGCATTACCAGCCCTATGCCCCACCACGCGACTTCG |
| | | CAGCCTATCGCTCCctgagagtgaagttcagcaggagcgcagacgccc |
| | | ccgcgtaccagcagggccagaaccagctctataacgagctcaatctaggacgaaga |
| | | gaggagtacgatgttttggacaagagacgtggccgggaccctgagatggggggaa |
| | | agccgagaaggaagaaccctcaggaaggcctgtacaatgaactgcagaaagataa |
| | | gatggcggaggcctacagtgagattgggatgaaaggcgagcgccggaggggcaa |
| | | ggggcacgatggcctttaccagggtctcagtacagccaccaaggacacctacgacg |
| | | cccttcacatgcaggccctgccccctcgcactagtggaagcggagctactaacttca |
| | | gcctgctgaagcaggctggagacgtggaggagaaccctggaccttctagaatggac |
| | | atggcggatgagccactcaatggaagccacacatggctatccattccatttgacctca |
| | | atggctctgtggtgtcaaccaacacctcaaaccagacagagccgtactatgacctgac |
| | | aagcaatgcagtcctcacattcatctattttgtggtctgcatcattgggttgtgtggcaac |
| | | acacttgtcatttatgtcatcctccgctatgccaagatgaagaccatcaccaacatttac |
| | | atcctcaacctggccatcgcagatgagctcttcatgctgggtctgcctttcttggctatg |
| | | caggtggctctggtccactggccctttggcaaggccatttgccgggtggtcatgactg |
| | | tggatggcatcaatcagttcaccagcatcttctgcctgacagtcatgagcatcgaccga |
| | | tacctggctgtggtccaccccatcaagtcggccaagtggaggagaccccggacggc |
| | | caagatgatcaccatggctgtgtggggagtctctctgctggtcatcttgcccatcatgat |
| | | atatgctgggctccggagcaaccagtgggggagaagcagctgcaccatcaactggc |
| | | caggtgaatctggggcttggtacacagggttcatcatctacactttcattctggggttcc |
| | | tggtacccctcaccatcatctgtctttgctacctgttcattatcatcaaggtgaagtcctct |
| | | ggaatccgagtgggctcctctaagaggaagaagtctgagaagaaggtcacccgaat |
| | | ggtgtccatcgtggtggctgtcttcatcttctgctggcttcccttctacatattcaacgttt |
| | | cttccgtctccatggccatcagccccaccccagcccttaaaggcatgtttgactttgtggt |
| | | ggtcctcacctatgctaacagctgtgccaaccctatcctatatgccttcttgtctgacaac |
| | | ttcaagaagagcttccagaatgtcctctgcttggtcaaggtgagcggcacagatgatg |
| | | gggagcggagtgacagtaagcaggacaaatcccggctgaatgagaccacggaga |
| | | cccagaggaccctcctcaatggagacctccaaaccagtatctaa |
| 66 | EF1α-MycT-Affy7- | ggctccggtgcccgtcagtgggcagagcgcacatcgcccacagtccccgagaagtt |
| | Y6V CAR-SSTR2 | ggggggaggggtcggcaattgaaccggtgcctagagaaggtggcgcggggtaaa |
| | | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtgggtgggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cggggggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagtt |
| | | tttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCCAGCAGAGCGGCCCTGAGCTGGTGCGCCCCGG |
| | | CGCTTCCGTGAAGATGTCATGCCGAGCTTCTGGCTA |
| | | CACGTTCACCAGCTATTGGCTACACTGGGTCAAGCA |
| | | GAGGCCCGGACAGGGTCTGGAGTGGATCGGTATGA |
| | | TTGACCCGTCAAATAGTGACACCCGCTTCAACCCCA |
| | | ACTTCAAGGACAAGGCCACTCTCAACGTGGACCGC |
| | | TCGAGCAACACGGCCTACATGCTGCTGTCCTCTCTG |
| | | ACCTCGGCGGACAGCGCCGTGTACTACTGTGCCACC |
| | | GTCGGCTCCTACGTGAGCCCTTTGGATTATTGGGGCC |
| | | AGGGCACCTCCGTCACCGTGTCCTCCggtggaggcggttca |
| | | ggcggaggtggctctggcggtggcggatcgGACATCATGATGTCTC |
| | | AGAGTCCCTCTTCTCTGACCGTGTCCGTGGGCGAGA |
| | | AGGTGACCGTGTCATGCAAGTCCTCACAGAGCCTTC |
| | | TGTACACCAGCTCCCAGAAGAACTACCTGGCGTGG |
| | | TACCAGCAGAAGCCCGGACAGAGCCCCAAGCTGCT |
| | | GATCTATTGGGCTTCCACCCGCGAGAGCGGCGTCCC |
| | | CGACCGCTTCACCGGCTCCGGCTCCGGGACCGACTT |
| | | CACCCTGACCATCACCTCCGTGAAGGCCGATGACCT |
| | | GGCCGTGTACTACTGTCAACAGTATTACGCCTACCC |
| | | GTGGACCTTCGGAGGCGGCACCAAGCTGGAGATCA |
| | | AGCGCgctagcacgaccactccggcgccgcgcccaccgactccggccccaac |
| | | tatcgcgagccagcccctgtcgctgaggccggaagcatgccgccctgccgccgga |
| | | ggtgctgtgcatacccggggattggacttcgcatgcgacTTTTGGGTGCTG |
| | | GTGGTGGTTGGTGGAGTCCTGGCTTGCTATAGCTTG |
| | | CTAGTAACAGTGGCCTTTATTATTTTCTGGGTGAGG |
| | | AGTAAGAGGAGCAGGCTCCTGCACAGTGACTACAT |
| | | GAACATGACTCCCCGCCGCCCCGGGCCCACCCGCA |
| | | AGCATTACCAGCCCTATGCCCCACCACGCGACTTCG |
| | | CAGCCTATCGCTCCctgagagtgaagttcagcaggagcgcagacgccc |
| | | ccgcgtaccagcagggccagaaccagctctataacgagctcaatctaggacgaaga |
| | | gaggagtacgatgttttggacaagagacgtggccgggaccctgagatggggggaa |
| | | agccgagaaggaagaaccctcaggaaggcctgtacaatgaactgcagaaagataa |
| | | gatggcggaggcctacagtgagattgggatgaaaggcgagcgccggaggggcaa |
| | | ggggcacgatggcctttaccagggtctcagtacagccaccaaggacacctacgacg |
| | | cccttcacatgcaggccctgccccctcgcactagtggaagcggagctactaacttca |
| | | gcctgctgaagcaggctggagacgtggaggagaaccctggaccttctagaatggac |
| | | atggcggatgagccactcaatggaagccacacatggctatccattccatttgacctca |
| | | atggctctgtggtgtcaaccaacacctcaaaccagacagagccgtactatgacctgac |
| | | aagcaatgcagtcctcacattcatctattttgtggtctgcatcattgggttgtgtggcaac |
| | | acacttgtcatttatgtcatcctccgctatgccaagatgaagaccatcaccaacatttac |
| | | atcctcaacctggccatcgcagatgagctcttcatgctgggtctgcctttcttggctatg |
| | | caggtggctctggtccactggccctttggcaaggccatttgccgggtggtcatgactg |
| | | tggatggcatcaatcagttcaccagcatcttctgcctgacagtcatgagcatcgaccga |
| | | tacctggctgtggtccaccccatcaagtcggccaagtggaggagaccccggacggc |
| | | caagatgatcaccatggctgtgtggggagtctctctgctggtcatcttgcccatcatgat |
| | | atatgctgggctccggagcaaccagtgggggagaagcagctgcaccatcaactggc |
| | | caggtgaatctggggcttggtacacagggttcatcatctacactttcattctggggttcc |
| | | tggtacccctcaccatcatctgtctttgctacctgttcattatcatcaaggtgaagtcctct |
| | | ggaatccgagtgggctcctctaagaggaagaagtctgagaagaaggtcacccgaat |
| | | ggtgtccatcgtggtggctgtcttcatcttctgctggcttcccttctacatattcaacgtttc |
| | | ttccgtctccatggccatcagccccaccccagcccttaaaggcatgtttgactttgtggt |
| | | ggtcctcacctatgctaacagctgtgccaaccctatcctatatgccttcttgtctgacaac |
| | | ttcaagaagagcttccagaatgtcctctgcttggtcaaggtgagcggcacagatgatg |
| | | gggagcggagtgacagtaagcaggacaaatcccggctgaatgagaccacggaga |
| | | cccagaggaccctcctcaatggagacctccaaaccagtatctaa |
| 67 | EF1α-MycT-Affy7- | ggctccggtgcccgtcagtgggcagagcgcacatcgcccacagtccccgagaagtt |
| | Y6L CAR-SSTR2 | ggggggaggggtcggcaattgaaccggtgcctagagaaggtggcgcggggtaaa |
| | | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtgggtgggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagttt |
| | | ttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCCAGCAGAGCGGCCCTGAGCTGGTGCGCCCCGG |
| | | CGCTTCCGTGAAGATGTCATGCCGAGCTTCTGGCTA |
| | | CACGTTCACCAGCTATTGGCTACACTGGGTCAAGCA |
| | | GAGGCCCGGACAGGGTCTGGAGTGGATCGGTATGA |
| | | TTGACCCGTCAAATAGTGACACCCGCTTCAACCCCA |
| | | ACTTCAAGGACAAGGCCACTCTCAACGTGGACCGC |
| | | TCGAGCAACACGGCCTACATGCTGCTGTCCTCTCTG |
| | | ACCTCGGCGGACAGCGCCGTGTACTACTGTGCCACC |
| | | CTCGGCTCCTACGTGAGCCCTTTGGATTATTGGGGCC |
| | | AGGGCACCTCCGTCACCGTGTCCTCCggtggaggcggttca |
| | | ggcggaggtggctctggcggtggcggatcgGACATCATGATGTCTC |
| | | AGAGTCCCTCTTCTCTGACCGTGTCCGTGGGCGAGA |
| | | AGGTGACCGTGTCATGCAAGTCCTCACAGAGCCTTC |
| | | TGTACACCAGCTCCCAGAAGAACTACCTGGCGTGG |
| | | TACCAGCAGAAGCCCGGACAGAGCCCCAAGCTGCT |
| | | GATCTATTGGGCTTCCACCCGCGAGAGCGGCGTCCC |
| | | CGACCGCTTCACCGGCTCCGGCTCCGGGACCGACTT |
| | | CACCCTGACCATCACCTCCGTGAAGGCCGATGACCT |
| | | GGCCGTGTACTACTGTCAACAGTATTACGCCTACCC |
| | | GTGGACCTTCGGAGGCGGCACCAAGCTGGAGATCA |
| | | AGCGCgctagcacgaccactccggcgccgcgcccaccgactccggccccaac |
| | | tatcgcgagccagcccctgtcgctgaggccggaagcatgccgccctgccgccgga |
| | | ggtgctgtgcatacccggggattggacttcgcatgcgacTTTTGGGTGCTG |
| | | GTGGTGGTTGGTGGAGTCCTGGCTTGCTATAGCTTG |
| | | CTAGTAACAGTGGCCTTTATTATTTTCTGGGTGAGG |
| | | AGTAAGAGGAGCAGGCTCCTGCACAGTGACTACAT |
| | | GAACATGACTCCCCGCCGCCCCGGGCCCACCCGCA |
| | | AGCATTACCAGCCCTATGCCCCACCACGCGACTTCG |
| | | CAGCCTATCGCTCCctgagagtgaagttcagcaggagcgcagacgccc |
| | | ccgcgtaccagcagggccagaaccagctctataacgagctcaatctaggacgaaga |
| | | gaggagtacgatgttttggacaagagacgtggccgggaccctgagatggggggaa |
| | | agccgagaaggaagaaccctcaggaaggcctgtacaatgaactgcagaaagataa |
| | | gatggcggaggcctacagtgagattgggatgaaaggcgagcgccggaggggcaa |
| | | ggggcacgatggcctttaccagggtctcagtacagccaccaaggacacctacgacg |
| | | cccttcacatgcaggccctgccccctcgcactagtggaagcggagctactaacttca |
| | | gcctgctgaagcaggctggagacgtggaggagaaccctggaccttctagaatggac |
| | | atggcggatgagccactcaatggaagccacacatggctatccattccatttgacctca |
| | | atggctctgtggtgtcaaccaacacctcaaaccagacagagccgtactatgacctgac |
| | | aagcaatgcagtcctcacattcatctattttgtggtctgcatcattgggttgtgtggcaac |
| | | acacttgtcatttatgtcatcctccgctatgccaagatgaagaccatcaccaacatttac |
| | | atcctcaacctggccatcgcagatgagctcttcatgctgggtctgcctttcttggctatg |
| | | caggtggctctggtccactggccctttggcaaggccatttgccgggtggtcatgactg |
| | | tggatggcatcaatcagttcaccagcatcttctgcctgacagtcatgagcatcgaccga |
| | | tacctggctgtggtccaccccatcaagtcggccaagtggaggagaccccggacggc |
| | | caagatgatcaccatggctgtgtggggagtctctctgctggtcatcttgcccatcatgat |
| | | atatgctgggctccggagcaaccagtgggggagaagcagctgcaccatcaactggc |
| | | caggtgaatctggggcttggtacacagggttcatcatctacactttcattctggggttcc |
| | | tggtacccctcaccatcatctgtctttgctacctgttcattatcatcaaggtgaagtcctct |
| | | ggaatccgagtgggctcctctaagaggaagaagtctgagaagaaggtcacccgaat |
| | | ggtgtccatcgtggtggctgtcttcatcttctgctggcttcccttctacatattcaacgtttc |
| | | ttccgtctccatggccatcagccccaccccagcccttaaaggcatgtttgactttgtggt |
| | | ggtcctcacctatgctaacagctgtgccaaccctatcctatatgccttcttgtctgacaac |
| | | ttcaagaagagcttccagaatgtcctctgcttggtcaaggtgagcggcacagatgatg |
| | | gggagcggagtgacagtaagcaggacaaatcccggctgaatgagaccacggaga |
| | | cccagaggaccctcctcaatggagacctccaaaccagtatctaa |
| 68 | EF1α-MycT-Affy7- | ggctccggtgcccgtcagtgggcagagcgcacatcgcccacagtccccgagaagtt |
| | Y6I CAR-SSTR2 | ggggggaggggtcggcaattgaaccggtgcctagagaaggtggcgcggggtaaa |
| | | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtgggtgggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagttt |
| | | ttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCCAGCAGAGCGGCCCTGAGCTGGTGCGCCCCGG |
| | | CGCTTCCGTGAAGATGTCATGCCGAGCTTCTGGCTA |
| | | CACGTTCACCAGCTATTGGCTACACTGGGTCAAGCA |
| | | GAGGCCCGGACAGGGTCTGGAGTGGATCGGTATGA |
| | | TTGACCCGTCAAATAGTGACACCCGCTTCAACCCCA |
| | | ACTTCAAGGACAAGGCCACTCTCAACGTGGACCGC |
| | | TCGAGCAACACGGCCTACATGCTGCTGTCCTCTCTG |
| | | ACCTCGGCGGACAGCGCCGTGTACTACTGTGCCACC |
| | | ATCGGCTCCTACGTGAGCCCTTTGGATTATTGGGGCC |
| | | AGGGCACCTCCGTCACCGTGTCCTCCggtggaggcggttca |
| | | ggcggaggtggctctggcggtggcggatcgGACATCATGATGTCTC |
| | | AGAGTCCCTCTTCTCTGACCGTGTCCGTGGGCGAGA |
| | | AGGTGACCGTGTCATGCAAGTCCTCACAGAGCCTTC |
| | | TGTACACCAGCTCCCAGAAGAACTACCTGGCGTGG |
| | | TACCAGCAGAAGCCCGGACAGAGCCCCAAGCTGCT |
| | | GATCTATTGGGCTTCCACCCGCGAGAGCGGCGTCCC |
| | | CGACCGCTTCACCGGCTCCGGCTCCGGGACCGACTT |
| | | CACCCTGACCATCACCTCCGTGAAGGCCGATGACCT |
| | | GGCCGTGTACTACTGTCAACAGTATTACGCCTACCC |
| | | GTGGACCTTCGGAGGCGGCACCAAGCTGGAGATCA |
| | | AGCGCgctagcacgaccactccggcgccgcgcccaccgactccggccccaac |
| | | tatcgcgagccagcccctgtcgctgaggccggaagcatgccgccctgccgccgga |
| | | ggtgctgtgcatacccggggattggacttcgcatgcgacTTTTGGGTGCTG |
| | | GTGGTGGTTGGTGGAGTCCTGGCTTGCTATAGCTTG |
| | | CTAGTAACAGTGGCCTTTATTATTTTCTGGGTGAGG |
| | | AGTAAGAGGAGCAGGCTCCTGCACAGTGACTACAT |
| | | GAACATGACTCCCCGCCGCCCCGGGCCCACCCGCA |
| | | AGCATTACCAGCCCTATGCCCCACCACGCGACTTCG |
| | | CAGCCTATCGCTCCctgagagtgaagttcagcaggagcgcagacgccc |
| | | ccgcgtaccagcagggccagaaccagctctataacgagctcaatctaggacgaaga |
| | | gaggagtacgatgttttggacaagagacgtggccgggaccctgagatggggggaa |
| | | agccgagaaggaagaaccctcaggaaggcctgtacaatgaactgcagaaagataa |
| | | gatggcggaggcctacagtgagattgggatgaaaggcgagcgccggaggggcaa |
| | | ggggcacgatggcctttaccagggtctcagtacagccaccaaggacacctacgacg |
| | | cccttcacatgcaggccctgccccctcgcactagtggaagcggagctactaacttca |
| | | gcctgctgaagcaggctggagacgtggaggagaaccctggaccttctagaatggac |
| | | atggcggatgagccactcaatggaagccacacatggctatccattccatttgacctca |
| | | atggctctgtggtgtcaaccaacacctcaaaccagacagagccgtactatgacctgac |
| | | aagcaatgcagtcctcacattcatctattttgtggtctgcatcattgggttgtgtggcaac |
| | | acacttgtcatttatgtcatcctccgctatgccaagatgaagaccatcaccaacatttac |
| | | atcctcaacctggccatcgcagatgagctcttcatgctgggtctgcctttcttggctatg |
| | | caggtggctctggtccactggccctttggcaaggccatttgccgggtggtcatgactg |
| | | tggatggcatcaatcagttcaccagcatcttctgcctgacagtcatgagcatcgaccga |
| | | tacctggctgtggtccaccccatcaagtcggccaagtggaggagaccccggacggc |
| | | caagatgatcaccatggctgtgtggggagtctctctgctggtcatcttgcccatcatgat |
| | | atatgctgggctccggagcaaccagtgggggagaagcagctgcaccatcaactggc |
| | | caggtgaatctggggcttggtacacagggttcatcatctacactttcattctggggttcc |
| | | tggtacccctcaccatcatctgtctttgctacctgttcattatcatcaaggtgaagtcctct |
| | | ggaatccgagtgggctcctctaagaggaagaagtctgagaagaaggtcacccgaat |
| | | ggtgtccatcgtggtggctgtcttcatcttctgctggcttcccttctacatattcaacgtttc |
| | | ttccgtctccatggccatcagccccaccccagcccttaaaggcatgtttgactttgtggt |
| | | ggtcctcacctatgctaacagctgtgccaaccctatcctatatgccttcttgtctgacaac |
| | | ttcaagaagagcttccagaatgtcctctgcttggtcaaggtgagcggcacagatgatg |
| | | gggagcggagtgacagtaagcaggacaaatcccggctgaatgagaccacggaga |
| | | cccagaggaccctcctcaatggagacctccaaaccagtatctaa |
| 69 | EF1α-MycT-Affy7- | ggctccggtgcccgtcagtgggcagagcgcacatcgcccacagtccccgagaagtt |
| | Y6M CAR-SSTR2 | ggggggaggggtcggcaattgaaccggtgcctagagaaggtggcgcggggtaaa |
| | | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtgggtgggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagttt |
| | | ttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCCAGCAGAGCGGCCCTGAGCTGGTGCGCCCCGG |
| | | CGCTTCCGTGAAGATGTCATGCCGAGCTTCTGGCTA |
| | | CACGTTCACCAGCTATTGGCTACACTGGGTCAAGCA |
| | | GAGGCCCGGACAGGGTCTGGAGTGGATCGGTATGA |
| | | TTGACCCGTCAAATAGTGACACCCGCTTCAACCCCA |
| | | ACTTCAAGGACAAGGCCACTCTCAACGTGGACCGC |
| | | TCGAGCAACACGGCCTACATGCTGCTGTCCTCTCTG |
| | | ACCTCGGCGGACAGCGCCGTGTACTACTGTGCCACC |
| | | ATGGGCTCCTACGTGAGCCCTTTGGATTATTGGGGCC |
| | | AGGGCACCTCCGTCACCGTGTCCTCCggtggaggcggttca |
| | | ggcggaggtggctctggcggtggcggatcgGACATCATGATGTCTC |
| | | AGAGTCCCTCTTCTCTGACCGTGTCCGTGGGCGAGA |
| | | AGGTGACCGTGTCATGCAAGTCCTCACAGAGCCTTC |
| | | TGTACACCAGCTCCCAGAAGAACTACCTGGCGTGG |
| | | TACCAGCAGAAGCCCGGACAGAGCCCCAAGCTGCT |
| | | GATCTATTGGGCTTCCACCCGCGAGAGCGGCGTCCC |
| | | CGACCGCTTCACCGGCTCCGGCTCCGGGACCGACTT |
| | | CACCCTGACCATCACCTCCGTGAAGGCCGATGACCT |
| | | GGCCGTGTACTACTGTCAACAGTATTACGCCTACCC |
| | | GTGGACCTTCGGAGGCGGCACCAAGCTGGAGATCA |
| | | AGCGCgctagcacgaccactccggcgccgcgcccaccgactccggccccaac |
| | | tatcgcgagccagcccctgtcgctgaggccggaagcatgccgccctgccgccgga |
| | | ggtgctgtgcatacccggggattggacttcgcatgcgacTTTTGGGTGCTG |
| | | GTGGTGGTTGGTGGAGTCCTGGCTTGCTATAGCTTG |
| | | CTAGTAACAGTGGCCTTTATTATTTTCTGGGTGAGG |
| | | AGTAAGAGGAGCAGGCTCCTGCACAGTGACTACAT |
| | | GAACATGACTCCCCGCCGCCCCGGGCCCACCCGCA |
| | | AGCATTACCAGCCCTATGCCCCACCACGCGACTTCG |
| | | CAGCCTATCGCTCCctgagagtgaagttcagcaggagcgcagacgccc |
| | | ccgcgtaccagcagggccagaaccagctctataacgagctcaatctaggacgaaga |
| | | gaggagtacgatgttttggacaagagacgtggccgggaccctgagatggggggaa |
| | | agccgagaaggaagaaccctcaggaaggcctgtacaatgaactgcagaaagataa |
| | | gatggcggaggcctacagtgagattgggatgaaaggcgagcgccggaggggcaa |
| | | ggggcacgatggcctttaccagggtctcagtacagccaccaaggacacctacgacg |
| | | cccttcacatgcaggccctgccccctcgcactagtggaagcggagctactaacttca |
| | | gcctgctgaagcaggctggagacgtggaggagaaccctggaccttctagaatggac |
| | | atggcggatgagccactcaatggaagccacacatggctatccattccatttgacctca |
| | | atggctctgtggtgtcaaccaacacctcaaaccagacagagccgtactatgacctgac |
| | | aagcaatgcagtcctcacattcatctattttgtggtctgcatcattgggttgtgtggcaac |
| | | acacttgtcatttatgtcatcctccgctatgccaagatgaagaccatcaccaacatttac |
| | | atcctcaacctggccatcgcagatgagctcttcatgctgggtctgcctttcttggctatg |
| | | caggtggctctggtccactggccctttggcaaggccatttgccgggtggtcatgactg |
| | | tggatggcatcaatcagttcaccagcatcttctgcctgacagtcatgagcatcgaccga |
| | | tacctggctgtggtccaccccatcaagtcggccaagtggaggagaccccggacggc |
| | | caagatgatcaccatggctgtgtggggagtctctctgctggtcatcttgcccatcatgat |
| | | atatgctgggctccggagcaaccagtgggggagaagcagctgcaccatcaactggc |
| | | caggtgaatctggggcttggtacacagggttcatcatctacactttcattctggggttcc |
| | | tggtacccctcaccatcatctgtctttgctacctgttcattatcatcaaggtgaagtcctct |
| | | ggaatccgagtgggctcctctaagaggaagaagtctgagaagaaggtcacccgaat |
| | | ggtgtccatcgtggtggctgtcttcatcttctgctggcttcccttctacatattcaacgtttc |
| | | ttccgtctccatggccatcagccccaccccagcccttaaaggcatgtttgactttgtggt |
| | | ggtcctcacctatgctaacagctgtgccaaccctatcctatatgccttcttgtctgacaac |
| | | ttcaagaagagcttccagaatgtcctctgcttggtcaaggtgagcggcacagatgatg |
| | | gggagcggagtgacagtaagcaggacaaatcccggctgaatgagaccacggaga |
| | | cccagaggaccctcctcaatggagacctccaaaccagtatctaa |
| 70 | Affy6 HCDR1 | GYIFTAY |
| 71 | Affy6 HCDR2 | LEWMGWIKPNNGLANY |
| 72 | Affy6-I3A HCDR3 | SEATTEFDY |
| 73 | Affy6-I3W HCDR3 | SEWTTEFDY |
| 74 | Affy6-I3F HCDR3 | SEFTTEFDY |
| 75 | Affy6-I3G HCDR3 | SEGTTEFDY |
| 76 | Affy6 LDR1 | ESVDSY |
| 77 | Affy6 LDR2 | PKLLIYRASTRE |
| 78 | Affy6 LDR3 | SKEDPL |
| 79 | Affy6 VH (wt) | QVQLVQSGAEVKKPGASVKVSCKASGYIFTAYTMHW |
| | | VRQAPGQGLEWMGWIKPNNGLANYAQKFQGRVTMTR |
| | | DTSISTAYMELSRLRSDDTAVYYCARSEITTEFDYWGQ |
| | | GTLVTVSS |
| 80 | Affy6-I3A VH | QVQLVQSGAEVKKPGASVKVSCKASGYIFTAYTMHW |
| | | VRQAPGQGLEWMGWIKPNNGLANYAQKFQGRVTMTR |
| | | DTSISTAYMELSRLRSDDTAVYYCARSEATTEFDYWG |
| | | QGTLVTVSS |
| 81 | Affy6-I3W VH | QVQLVQSGAEVKKPGASVKVSCKASGYIFTAYTMHW |
| | | VRQAPGQGLEWMGWIKPNNGLANYAQKFQGRVTMTR |
| | | DTSISTAYMELSRLRSDDTAVYYCARSEWTTEFDYWG |
| | | QGTLVTVSS |
| 82 | Affy6-I3F VH | QVQLVQSGAEVKKPGASVKVSCKASGYIFTAYTMHW |
| | | VRQAPGQGLEWMGWIKPNNGLANYAQKFQGRVTMTR |
| | | DTSISTAYMELSRLRSDDTAVYYCARSEFTTEFDYWG |
| | | QGTLVTVSS |
| 83 | Affy6-I3G VH | QVQLVQSGAEVKKPGASVKVSCKASGYIFTAYTMHW |
| | | VRQAPGQGLEWMGWIKPNNGLANYAQKFQGRVTMTR |
| | | DTSISTAYMELSRLRSDDTAVYYCARSEGTTEFDYWG |
| | | QGTLVTVSS |
| 84 | Affy6 VL (wt) | DIVLTQSPDSLAVSLGERATINCKSSESVDSYANSFMH |
| | | WYQQKPGQPPKLLIYRASTRESGVPDRFSGSGSRTDFTL |
| | | TISSLQAEDVAVYYCQQSKEDPLTFGGGTKVEIK |
| 85 | Affy6 VH (wt) | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACTG |
| | | GGTGCGCCAGGCCCCGGGACAGGGGCTGGAGTGGA |
| | | TGGGCTGGATCAAGCCTAACAACGGTCTGGCCAACTAC |
| | | GCGCAGAAGTTCCAGGGCCGCGTGACCATGACTCG |
| | | GGACACCAGCATCTCTACCGCGTACATGGAGCTGTC |
| | | CCGCCTGCGCTCCGATGACACGGCCGTGTACTACTG |
| | | CGCCCGCTCGGAGATCACCACCGAGTTCGACTATTGG |
| | | GGCCAGGGCACCCTGGTGACCGTGTCGTCC |
| 86 | Affy6-I3A VH | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACTG |
| | | GGTGCGCCAGGCCCCGGGACAGGGGCTGGAGTGGA |
| | | TGGGCTGGATCAAGCCTAACAACGGTCTGGCCAACTAC |
| | | GCGCAGAAGTTCCAGGGCCGCGTGACCATGACTCG |
| | | GGACACCAGCATCTCTACCGCGTACATGGAGCTGTC |
| | | CCGCCTGCGCTCCGATGACACGGCCGTGTACTACTG |
| | | CGCCCGCTCGGAGGCCACCACCGAGTTCGACTATTGG |
| | | GGCCAGGGCACCCTGGTGACCGTGTCGTCC |
| 87 | Affy6-I3W VH | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACTG |
| | | GGTGCGCCAGGCCCCGGGACAGGGGCTGGAGTGGA |
| | | TGGGCTGGATCAAGCCTAACAACGGTCTGGCCAACTAC |
| | | GCGCAGAAGTTCCAGGGCCGCGTGACCATGACTCG |
| | | GGACACCAGCATCTCTACCGCGTACATGGAGCTGTC |
| | | CCGCCTGCGCTCCGATGACACGGCCGTGTACTACTG |
| | | CGCCCGCTCGGAGTGGACCACCGAGTTCGACTATTGG |
| | | GGCCAGGGCACCCTGGTGACCGTGTCGTCC |
| 88 | Affy6-I3F VH | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACTG |
| | | GGTGCGCCAGGCCCCGGGACAGGGGCTGGAGTGGA |
| | | TGGGCTGGATCAAGCCTAACAACGGTCTGGCCAACTAC |
| | | GCGCAGAAGTTCCAGGGCCGCGTGACCATGACTCG |
| | | GGACACCAGCATCTCTACCGCGTACATGGAGCTGTC |
| | | CCGCCTGCGCTCCGATGACACGGCCGTGTACTACTG |
| | | CGCCCGCTCGGAGTTCACCACCGAGTTCGACTATTGG |
| | | GGCCAGGGCACCCTGGTGACCGTGTCGTCC |
| 89 | Affy6-I3G VH | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACTG |
| | | GGTGCGCCAGGCCCCGGGACAGGGGCTGGAGTGGA |
| | | TGGGCTGGATCAAGCCTAACAACGGTCTGGCCAACTAC |
| | | GCGCAGAAGTTCCAGGGCCGCGTGACCATGACTCG |
| | | GGACACCAGCATCTCTACCGCGTACATGGAGCTGTC |
| | | CCGCCTGCGCTCCGATGACACGGCCGTGTACTACTG |
| | | CGCCCGCTCGGAGGGCACCACCGAGTTCGACTATTGG |
| | | GGCCAGGGCACCCTGGTGACCGTGTCGTCC |
| 90 | Affy6 VL (wt) | GACATCGTGCTGACCCAGAGCCCTGACAGCCTGGC |
| | | CGTGTCCCTGGGCGAGCGCGCCACCATTAACTGCA |
| | | AGAGCTCCGAGAGTGTCGATAGCTACGCCAACTCCTT |
| | | CATGCACTGGTACCAGCAGAAGCCCGGCCAGCCAC |
| | | CCAAGCTGCTGATCTACAGGGCTTCCACCCGCGAGAG |
| | | CGGCGTCCCCGACAGGTTTTCAGGTTCTGGCTCCCG |
| | | CACCGACTTCACCCTGACCATCTCTTCTCTGCAGGC |
| | | CGAGGATGTGGCGGTGTACTACTGTCAACAGTCCAA |
| | | GGAGGACCCGCTTACCTTCGGCGGCGGCACCAAGGT |
| | | GGAGATCAAG |
| 91 | Affy6 scFv (control) | QVQLVQSGAEVKKPGASVKVSCKASGYIFTAYTMHW |
| | | VRQAPGQGLEWMGWIKPNNGLANYAQKFQGRVTMT |
| | | RDTSISTAYMELSRLRSDDTAVYYCARSEITTEFDYWG |
| | | QGTLVTVSSGGGGSGGGGSGGGGSDIVLTQSPDSLAV |
| | | SLGERATINCKSSESVDSYANSFMHWYQQKPGQPPKL |
| | | LIYRASTRESGVPDRFSGSGSRTDFTLTISSLQAEDVAV |
| | | YYCQQSKEDPLTFGGGTKVEIK |
| 92 | Affy6-I3A scFv | QVQLVQSGAEVKKPGASVKVSCKASGYIFTAYTMHW |
| | | VRQAPGQGLEWMGWIKPNNGLANYAQKFQGRVTMT |
| | | RDTSISTAYMELSRLRSDDTAVYYCARSEATTEFDYW |
| | | GQGTLVTVSSGGGGSGGGGSGGGGSDIVLTQSPDSLA |
| | | VSLGERATINCKSSESVDSYANSFMHWYQQKPGQPPK |
| | | LLIYRASTRESGVPDRFSGSGSRTDFTLTISSLQAEDVA |
| | | VYYCQQSKEDPLTFGGGTKVEIK |
| 93 | Affy6-I3W scFv | QVQLVQSGAEVKKPGASVKVSCKASGYIFTAYTMHW |
| | | VRQAPGQGLEWMGWIKPNNGLANYAQKFQGRVTMT |
| | | RDTSISTAYMELSRLRSDDTAVYYCARSEWTTEFDYW |
| | | GQGTLVTVSSGGGGSGGGGSGGGGSDIVLTQSPDSLA |
| | | VSLGERATINCKSSESVDSYANSFMHWYQQKPGQPPK |
| | | LLIYRASTRESGVPDRFSGSGSRTDFTLTISSLQAEDVA |
| | | VYYCQQSKEDPLTFGGGTKVEIK |
| 94 | Affy6-I3F scFv | QVQLVQSGAEVKKPGASVKVSCKASGYIFTAYTMHW |
| | | VRQAPGQGLEWMGWIKPNNGLANYAQKFQGRVTMT |
| | | RDTSISTAYMELSRLRSDDTAVYYCARSEFTTEFDYW |
| | | GQGTLVTVSSGGGGSGGGGSGGGGSDIVLTQSPDSLA |
| | | VSLGERATINCKSSESVDSYANSFMHWYQQKPGQPPK |
| | | LLIYRASTRESGVPDRFSGSGSRTDFTLTISSLQAEDVA |
| | | VYYCQQSKEDPLTFGGGTKVEIK |
| 95 | Affy6-I3G scFv | QVQLVQSGAEVKKPGASVKVSCKASGYIFTAYTMHW |
| | | VRQAPGQGLEWMGWIKPNNGLANYAQKFQGRVTMT |
| | | RDTSISTAYMELSRLRSDDTAVYYCARSEGTTEFDYW |
| | | GQGTLVTVSSGGGGSGGGGSGGGGSDIVLTQSPDSLA |
| | | VSLGERATINCKSSESVDSYANSFMHWYQQKPGQPPK |
| | | LLIYRASTRESGVPDRFSGSGSRTDFTLTISSLQAEDVA |
| | | VYYCQQSKEDPLTFGGGTKVEIK |
| 96 | Affy6 scFv (control) | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACT |
| | | GGGTGCGCCAGGCCCCGGGACAGGGGCTGGAGTGG |
| | | ATGGGCTGGATCAAGCCTAACAACGGTCTGGCCAA |
| | | CTACGCGCAGAAGTTCCAGGGCCGCGTGACCATGA |
| | | CTCGGGACACCAGCATCTCTACCGCGTACATGGAG |
| | | CTGTCCCGCCTGCGCTCCGATGACACGGCCGTGTAC |
| | | TACTGCGCCCGCTCGGAGATCACCACCGAGTTCGACT |
| | | ATTGGGGCCAGGGCACCCTGGTGACCGTGTCGTCCG |
| | | GTGGAGGCGGTTCAGGCGGAGGTGGCTCTGGCGGT |
| | | GGCGGATCGGACATCGTGCTGACCCAGAGCCCTGA |
| | | CAGCCTGGCCGTGTCCCTGGGCGAGCGCGCCACCA |
| | | TTAACTGCAAGAGCTCCGAGAGTGTCGATAGCTAC |
| | | GCCAACTCCTTCATGCACTGGTACCAGCAGAAGCCC |
| | | GGCCAGCCACCCAAGCTGCTGATCTACAGGGCTTCC |
| | | ACCCGCGAGAGCGGCGTCCCCGACAGGTTTTCAGG |
| | | TTCTGGCTCCCGCACCGACTTCACCCTGACCATCTC |
| | | TTCTCTGCAGGCCGAGGATGTGGCGGTGTACTACTG |
| | | TCAACAGTCCAAGGAGGACCCGCTTACCTTCGGCG |
| | | GCGGCACCAAGGTGGAGATCAAG |
| 97 | Affy6-I3A scFv | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACT |
| | | GGGTGCGCCAGGCCCCGGGACAGGGGCTGGAGTGG |
| | | ATGGGCTGGATCAAGCCTAACAACGGTCTGGCCAA |
| | | CTACGCGCAGAAGTTCCAGGGCCGCGTGACCATGA |
| | | CTCGGGACACCAGCATCTCTACCGCGTACATGGAG |
| | | CTGTCCCGCCTGCGCTCCGATGACACGGCCGTGTAC |
| | | TACTGCGCCCGCTCGGAGGCCACCACCGAGTTCGACT |
| | | ATTGGGGCCAGGGCACCCTGGTGACCGTGTCGTCCG |
| | | GTGGAGGCGGTTCAGGCGGAGGTGGCTCTGGCGGT |
| | | GGCGGATCGGACATCGTGCTGACCCAGAGCCCTGA |
| | | CAGCCTGGCCGTGTCCCTGGGCGAGCGCGCCACCA |
| | | TTAACTGCAAGAGCTCCGAGAGTGTCGATAGCTAC |
| | | GCCAACTCCTTCATGCACTGGTACCAGCAGAAGCCC |
| | | GGCCAGCCACCCAAGCTGCTGATCTACAGGGCTTCC |
| | | ACCCGCGAGAGCGGCGTCCCCGACAGGTTTTCAGG |
| | | TTCTGGCTCCCGCACCGACTTCACCCTGACCATCTC |
| | | TTCTCTGCAGGCCGAGGATGTGGCGGTGTACTACTG |
| | | TCAACAGTCCAAGGAGGACCCGCTTACCTTCGGCG |
| | | GCGGCACCAAGGTGGAGATCAAG |
| 98 | Affy6-I3W scFv | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACT |
| | | GGGTGCGCCAGGCCCCGGGACAGGGGCTGGAGTGG |
| | | ATGGGCTGGATCAAGCCTAACAACGGTCTGGCCAA |
| | | CTACGCGCAGAAGTTCCAGGGCCGCGTGACCATGA |
| | | CTCGGGACACCAGCATCTCTACCGCGTACATGGAG |
| | | CTGTCCCGCCTGCGCTCCGATGACACGGCCGTGTAC |
| | | TACTGCGCCCGCTCGGAGTGGACCACCGAGTTCGACT |
| | | ATTGGGGCCAGGGCACCCTGGTGACCGTGTCGTCCG |
| | | GTGGAGGCGGTTCAGGCGGAGGTGGCTCTGGCGGT |
| | | GGCGGATCGGACATCGTGCTGACCCAGAGCCCTGA |
| | | CAGCCTGGCCGTGTCCCTGGGCGAGCGCGCCACCA |
| | | TTAACTGCAAGAGCTCCGAGAGTGTCGATAGCTAC |
| | | GCCAACTCCTTCATGCACTGGTACCAGCAGAAGCCC |
| | | GGCCAGCCACCCAAGCTGCTGATCTACAGGGCTTCC |
| | | ACCCGCGAGAGCGGCGTCCCCGACAGGTTTTCAGG |
| | | TTCTGGCTCCCGCACCGACTTCACCCTGACCATCTC |
| | | TTCTCTGCAGGCCGAGGATGTGGCGGTGTACTACTG |
| | | TCAACAGTCCAAGGAGGACCCGCTTACCTTCGGCG |
| | | GCGGCACCAAGGTGGAGATCAAG |
| 99 | Affy6-I3F scFv | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACT |
| | | GGGTGCGCCAGGCCCCGGGACAGGGGCTGGAGTGG |
| | | ATGGGCTGGATCAAGCCTAACAACGGTCTGGCCAA |
| | | CTACGCGCAGAAGTTCCAGGGCCGCGTGACCATGA |
| | | CTCGGGACACCAGCATCTCTACCGCGTACATGGAG |
| | | CTGTCCCGCCTGCGCTCCGATGACACGGCCGTGTAC |
| | | TACTGCGCCCGCTCGGAGTTCACCACCGAGTTCGACT |
| | | ATTGGGGCCAGGGCACCCTGGTGACCGTGTCGTCCG |
| | | GTGGAGGCGGTTCAGGCGGAGGTGGCTCTGGCGGT |
| | | GGCGGATCGGACATCGTGCTGACCCAGAGCCCTGA |
| | | CAGCCTGGCCGTGTCCCTGGGCGAGCGCGCCACCA |
| | | TTAACTGCAAGAGCTCCGAGAGTGTCGATAGCTAC |
| | | GCCAACTCCTTCATGCACTGGTACCAGCAGAAGCCC |
| | | GGCCAGCCACCCAAGCTGCTGATCTACAGGGCTTCC |
| | | ACCCGCGAGAGCGGCGTCCCCGACAGGTTTTCAGG |
| | | TTCTGGCTCCCGCACCGACTTCACCCTGACCATCTC |
| | | TTCTCTGCAGGCCGAGGATGTGGCGGTGTACTACTG |
| | | TCAACAGTCCAAGGAGGACCCGCTTACCTTCGGCG |
| | | GCGGCACCAAGGTGGAGATCAAG |
| 100 | Affy6-I3G scFv | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACT |
| | | GGGTGCGCCAGGCCCCGGGACAGGGGCTGGAGTGG |
| | | ATGGGCTGGATCAAGCCTAACAACGGTCTGGCCAA |
| | | CTACGCGCAGAAGTTCCAGGGCCGCGTGACCATGA |
| | | CTCGGGACACCAGCATCTCTACCGCGTACATGGAG |
| | | CTGTCCCGCCTGCGCTCCGATGACACGGCCGTGTAC |
| | | TACTGCGCCCGCTCGGAGGGCACCACCGAGTTCGACT |
| | | ATTGGGGCCAGGGCACCCTGGTGACCGTGTCGTCCG |
| | | GTGGAGGCGGTTCAGGCGGAGGTGGCTCTGGCGGT |
| | | GGCGGATCGGACATCGTGCTGACCCAGAGCCCTGA |
| | | CAGCCTGGCCGTGTCCCTGGGCGAGCGCGCCACCA |
| | | TTAACTGCAAGAGCTCCGAGAGTGTCGATAGCTAC |
| | | GCCAACTCCTTCATGCACTGGTACCAGCAGAAGCCC |
| | | GGCCAGCCACCCAAGCTGCTGATCTACAGGGCTTCC |
| | | ACCCGCGAGAGCGGCGTCCCCGACAGGTTTTCAGG |
| | | TTCTGGCTCCCGCACCGACTTCACCCTGACCATCTC |
| | | TTCTCTGCAGGCCGAGGATGTGGCGGTGTACTACTG |
| | | TCAACAGTCCAAGGAGGACCCGCTTACCTTCGGCG |
| | | GCGGCACCAAGGTGGAGATCAAG |
| 101 | Affy6 CAR (control) | QVQLVQSGAEVKKPGASVKVSCKASGYIFTAYTMHW |
| | | VRQAPGQGLEWMGWIKPNNGLANYAQKFQGRVTMT |
| | | RDTSISTAYMELSRLRSDDTAVYYCARSEITTEFDYWG |
| | | QGTLVTVSSGGGGSGGGGSGGGGSDIVLTQSPDSLAV |
| | | SLGERATINCKSSESVDSYANSFMHWYQQKPGQPPKL |
| | | LIYRASTRESGVPDRFSGSGSRTDFTLTISSLQAEDVAV |
| | | YYCQQSKEDPLTFGGGTKVEIKASTTTPAPRPPTPAPTI |
| | | ASQPLSLRPEACRPAAGGAVHTRGLDFACDFWVLVV |
| | | VGGVLACYSLLVTVAFIIFWVRSKRSRLLHSDYMNMT |
| | | PRRPGPTRKHYQPYAPPRDFAAYRSLRVKFSRSADAP |
| | | AYQQGQNQLYNELNLGRREEYDVLDKRRGRDPEMG |
| | | GKPRRKNPQEGLYNELQKDKMAEAYSEIGMKGERRR |
| | | GKGHDGLYQGLSTATKDTYDALHMQALPPR |
| 102 | Affy6-I3A CAR | QVQLVQSGAEVKKPGASVKVSCKASGYIFTAYTMHW |
| | | VRQAPGQGLEWMGWIKPNNGLANYAQKFQGRVTMT |
| | | RDTSISTAYMELSRLRSDDTAVYYCARSEATTEFDYW |
| | | GQGTLVTVSSGGGGSGGGGSGGGGSDIVLTQSPDSLA |
| | | VSLGERATINCKSSESVDSYANSFMHWYQQKPGQPPK |
| | | LLIYRASTRESGVPDRFSGSGSRTDFTLTISSLQAEDVA |
| | | VYYCQQSKEDPLTFGGGTKVEIKASTTTPAPRPPTPAP |
| | | TIASQPLSLRPEACRPAAGGAVHTRGLDFACDFWVLV |
| | | VVGGVLACYSLLVTVAFIIFWVRSKRSRLLHSDYMN |
| | | MTPRRPGPTRKHYQPYAPPRDFAAYRSLRVKFSRSAD |
| | | APAYQQGQNQLYNELNLGRREEYDVLDKRRGRDPE |
| | | MGGKPRRKNPQEGLYNELQKDKMAEAYSEIGMKGE |
| | | RRRGKGHDGLYQGLSTATKDTYDALHMQALPPR |
| 103 | Affy6-I3W CAR | QVQLVQSGAEVKKPGASVKVSCKASGYIFTAYTMHW |
| | | VRQAPGQGLEWMGWIKPNNGLANYAQKFQGRVTMT |
| | | RDTSISTAYMELSRLRSDDTAVYYCARSEWTTEFDYW |
| | | GQGTLVTVSSGGGGSGGGGSGGGGSDIVLTQSPDSLA |
| | | VSLGERATINCKSSESVDSYANSFMHWYQQKPGQPPK |
| | | LLIYRASTRESGVPDRFSGSGSRTDFTLTISSLQAEDVA |
| | | VYYCQQSKEDPLTFGGGTKVEIKASTTTPAPRPPTPAP |
| | | TIASQPLSLRPEACRPAAGGAVHTRGLDFACDFWVLV |
| | | VVGGVLACYSLLVTVAFIIFWVRSKRSRLLHSDYMN |
| | | MTPRRPGPTRKHYQPYAPPRDFAAYRSLRVKFSRSAD |
| | | APAYQQGQNQLYNELNLGRREEYDVLDKRRGRDPE |
| | | MGGKPRRKNPQEGLYNELQKDKMAEAYSEIGMKGE |
| | | RRRGKGHDGLYQGLSTATKDTYDALHMQALPPR |
| 104 | Affy6-I3F CAR | QVQLVQSGAEVKKPGASVKVSCKASGYIFTAYTMHW |
| | | VRQAPGQGLEWMGWIKPNNGLANYAQKFQGRVTMT |
| | | RDTSISTAYMELSRLRSDDTAVYYCARSEFTTEFDYW |
| | | GQGTLVTVSSGGGGSGGGGSGGGGSDIVLTQSPDSLA |
| | | VSLGERATINCKSSESVDSYANSFMHWYQQKPGQPPK |
| | | LLIYRASTRESGVPDRFSGSGSRTDFTLTISSLQAEDVA |
| | | VYYCQQSKEDPLTFGGGTKVEIKASTTTPAPRPPTPAP |
| | | TIASQPLSLRPEACRPAAGGAVHTRGLDFACDFWVLV |
| | | VVGGVLACYSLLVTVAFIIFWVRSKRSRLLHSDYMN |
| | | MTPRRPGPTRKHYQPYAPPRDFAAYRSLRVKFSRSAD |
| | | APAYQQGQNQLYNELNLGRREEYDVLDKRRGRDPE |
| | | MGGKPRRKNPQEGLYNELQKDKMAEAYSEIGMKGE |
| | | RRRGKGHDGLYQGLSTATKDTYDALHMQALPPR |
| 105 | Affy6-I3G CAR | QVQLVQSGAEVKKPGASVKVSCKASGYIFTAYTMHW |
| | | VRQAPGQGLEWMGWIKPNNGLANYAQKFQGRVTMT |
| | | RDTSISTAYMELSRLRSDDTAVYYCARSEGTTEFDYW |
| | | GQGTLVTVSSGGGGSGGGGSGGGGSDIVLTQSPDSLA |
| | | VSLGERATINCKSSESVDSYANSFMHWYQQKPGQPPK |
| | | LLIYRASTRESGVPDRFSGSGSRTDFTLTISSLQAEDVA |
| | | VYYCQQSKEDPLTFGGGTKVEIKASTTTPAPRPPTPAP |
| | | TIASQPLSLRPEACRPAAGGAVHTRGLDFACDFWVLV |
| | | VVGGVLACYSLLVTVAFIIFWVRSKRSRLLHSDYMN |
| | | MTPRRPGPTRKHYQPYAPPRDFAAYRSLRVKFSRSAD |
| | | APAYQQGQNQLYNELNLGRREEYDVLDKRRGRDPE |
| | | MGGKPRRKNPQEGLYNELQKDKMAEAYSEIGMKGE |
| | | RRRGKGHDGLYQGLSTATKDTYDALHMQALPPR |
| 106 | MycT-Affy6 CAR- | GSEQKLISEEDLGSQVQLVQSGAEVKKPGASVKVSCK |
| | SSTR2 (control) | ASGYIFTAYTMHWVRQAPGQGLEWMGWIKPNNGLA |
| | | NYAQKFQGRVTMTRDTSISTAYMELSRLRSDDTAVY |
| | | YCARSEITTEFDYWGQGTLVTVSSGGGGSGGGGSGGG |
| | | GSDIVLTQSPDSLAVSLGERATINCKSSESVDSYANSF |
| | | MHWYQQKPGQPPKLLIYRASTRESGVPDRFSGSGSRT |
| | | DFTLTISSLQAEDVAVYYCQQSKEDPLTFGGGTKVEIK |
| | | ASTTTPAPRPPTPAPTIASQPLSLRPEACRPAAGGAVHT |
| | | RGLDFACDFWVLVVVGGVLACYSLLVTVAFIIFWVRS |
| | | KRSRLLHSDYMNMTPRRPGPTRKHYQPYAPPRDFAA |
| | | YRSLRVKFSRSADAPAYQQGQNQLYNELNLGRREEY |
| | | DVLDKRRGRDPEMGGKPRRKNPQEGLYNELQKDKM |
| | | AEAYSEIGMKGERRRGKGHDGLYQGLSTATKDTYDA |
| | | LHMQALPPRTSGSGATNFSLLKQAGDVEENPGPSRMD |
| | | MADEPLNGSHTWLSIPFDLNGSVVSTNTSNQTEPYYD |
| | | LTSNAVLTFIYFVVCIIGLCGNTLVIYVILRYAKMKTIT |
| | | NIYILNLAIADELFMLGLPFLAMQVALVHWPFGKAIC |
| | | RVVMTVDGINQFTSIFCLTVMSIDRYLAVVHPIKSAK |
| | | WRRPRTAKMITMAVWGVSLLVILPIMIYAGLRSNQW |
| | | GRSSCTINWPGESGAWYTGFIIYTFILGFLVPLTIICLCY |
| | | LFIIIKVKSSGIRVGSSKRKKSEKKVTRMVSIVVAVFIF |
| | | CWLPFYIFNVSSVSMAISPTPALKGMFDFVVVLTYAN |
| | | SCANPILYAFLSDNFKKSFQNVLCLVKVSGTDDGERS |
| | | DSKQDKSRLNETTETQRTLLNGDLQTSI |
| 107 | MycTAffy6-I3A | GSEQKLISEEDLGSQVQLVQSGAEVKKPGASVKVSCK |
| | CAR-SSTR2 | ASGYIFTAYTMHWVRQAPGQGLEWMGWIKPNNGLA |
| | | NYAQKFQGRVTMTRDTSISTAYMELSRLRSDDTAVY |
| | | YCARSEATTEFDYWGQGTLVTVSSGGGGSGGGGSGG |
| | | GGSDIVLTQSPDSLAVSLGERATINCKSSESVDSYANS |
| | | FMHWYQQKPGQPPKLLIYRASTRESGVPDRFSGSGSR |
| | | TDFTLTISSLQAEDVAVYYCQQSKEDPLTFGGGTKVEI |
| | | KASTTTPAPRPPTPAPTIASQPLSLRPEACRPAAGGAV |
| | | HTRGLDFACDFWVLVVVGGVLACYSLLVTVAFIIFW |
| | | VRSKRSRLLHSDYMNMTPRRPGPTRKHYQPYAPPRDF |
| | | AAYRSLRVKFSRSADAPAYQQGQNQLYNELNLGRRE |
| | | EYDVLDKRRGRDPEMGGKPRRKNPQEGLYNELQKD |
| | | KMAEAYSEIGMKGERRRGKGHDGLYQGLSTATKDTY |
| | | DALHMQALPPRTSGSGATNFSLLKQAGDVEENPGPSR |
| | | MDMADEPLNGSHTWLSIPFDLNGSVVSTNTSNQTEPY |
| | | YDLTSNAVLTFIYFVVCIIGLCGNTLVIYVILRYAKMK |
| | | TITNIYILNLAIADELFMLGLPFLAMQVALVHWPFGKA |
| | | ICRVVMTVDGINQFTSIFCLTVMSIDRYLAVVHPIKSA |
| | | KWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 108 | MycT-Affy6-I3W | GSEQKLISEEDLGSQVQLVQSGAEVKKPGASVKVSCK |
| | CAR-SSTR2 | ASGYIFTAYTMHWVRQAPGQGLEWMGWIKPNNGLA |
| | | NYAQKFQGRVTMTRDTSISTAYMELSRLRSDDTAVY |
| | | YCARSEWTTEFDYWGQGTLVTVSSGGGGSGGGGSGG |
| | | GGSDIVLTQSPDSLAVSLGERATINCKSSESVDSYANS |
| | | FMHWYQQKPGQPPKLLIYRASTRESGVPDRFSGSGSR |
| | | TDFTLTISSLQAEDVAVYYCQQSKEDPLTFGGGTKVEI |
| | | KASTTTPAPRPPTPAPTIASQPLSLRPEACRPAAGGAV |
| | | HTRGLDFACDFWVLVVVGGVLACYSLLVTVAFIIFW |
| | | VRSKRSRLLHSDYMNMTPRRPGPTRKHYQPYAPPRDF |
| | | AAYRSLRVKFSRSADAPAYQQGQNQLYNELNLGRRE |
| | | EYDVLDKRRGRDPEMGGKPRRKNPQEGLYNELQKD |
| | | KMAEAYSEIGMKGERRRGKGHDGLYQGLSTATKDTY |
| | | DALHMQALPPRTSGSGATNFSLLKQAGDVEENPGPSR |
| | | MDMADEPLNGSHTWLSIPFDLNGSVVSTNTSNQTEPY |
| | | YDLTSNAVLTFIYFVVCIIGLCGNTLVIYVILRYAKMK |
| | | TITNIYILNLAIADELFMLGLPFLAMQVALVHWPFGKA |
| | | ICRVVMTVDGINQFTSIFCLTVMSIDRYLAVVHPIKSA |
| | | KWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 109 | MycT-Affy6-I3F | GSEQKLISEEDLGSQVQLVQSGAEVKKPGASVKVSCK |
| | CAR-SSTR2 | ASGYIFTAYTMHWVRQAPGQGLEWMGWIKPNNGLA |
| | | NYAQKFQGRVTMTRDTSISTAYMELSRLRSDDTAVY |
| | | YCARSEFTTEFDYWGQGTLVTVSSGGGGSGGGGSGG |
| | | GGSDIVLTQSPDSLAVSLGERATINCKSSESVDSYANS |
| | | FMHWYQQKPGQPPKLLIYRASTRESGVPDRFSGSGSR |
| | | TDFTLTISSLQAEDVAVYYCQQSKEDPLTFGGGTKVEI |
| | | KASTTTPAPRPPTPAPTIASQPLSLRPEACRPAAGGAV |
| | | HTRGLDFACDFWVLVVVGGVLACYSLLVTVAFIIFW |
| | | VRSKRSRLLHSDYMNMTPRRPGPTRKHYQPYAPPRDF |
| | | AAYRSLRVKFSRSADAPAYQQGQNQLYNELNLGRRE |
| | | EYDVLDKRRGRDPEMGGKPRRKNPQEGLYNELQKD |
| | | KMAEAYSEIGMKGERRRGKGHDGLYQGLSTATKDTY |
| | | DALHMQALPPRTSGSGATNFSLLKQAGDVEENPGPSR |
| | | MDMADEPLNGSHTWLSIPFDLNGSVVSTNTSNQTEPY |
| | | YDLTSNAVLTFIYFVVCIIGLCGNTLVIYVILRYAKMK |
| | | TITNIYILNLAIADELFMLGLPFLAMQVALVHWPFGKA |
| | | ICRVVMTVDGINQFTSIFCLTVMSIDRYLAVVHPIKSA |
| | | KWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 110 | MycT-Affy6-I3G | GSEQKLISEEDLGSQVQLVQSGAEVKKPGASVKVSCK |
| | CAR-SSTR2 | ASGYIFTAYTMHWVRQAPGQGLEWMGWIKPNNGLA |
| | | NYAQKFQGRVTMTRDTSISTAYMELSRLRSDDTAVY |
| | | YCARSEGTTEFDYWGQGTLVTVSSGGGGSGGGGSGG |
| | | GGSDIVLTQSPDSLAVSLGERATINCKSSESVDSYANS |
| | | FMHWYQQKPGQPPKLLIYRASTRESGVPDRFSGSGSR |
| | | TDFTLTISSLQAEDVAVYYCQQSKEDPLTFGGGTKVEI |
| | | KASTTTPAPRPPTPAPTIASQPLSLRPEACRPAAGGAV |
| | | HTRGLDFACDFWVLVVVGGVLACYSLLVTVAFIIFW |
| | | VRSKRSRLLHSDYMNMTPRRPGPTRKHYQPYAPPRDF |
| | | AAYRSLRVKFSRSADAPAYQQGQNQLYNELNLGRRE |
| | | EYDVLDKRRGRDPEMGGKPRRKNPQEGLYNELQKD |
| | | KMAEAYSEIGMKGERRRGKGHDGLYQGLSTATKDTY |
| | | DALHMQALPPRTSGSGATNFSLLKQAGDVEENPGPSR |
| | | MDMADEPLNGSHTWLSIPFDLNGSVVSTNTSNQTEPY |
| | | YDLTSNAVLTFIYFVVCIIGLCGNTLVIYVILRYAKMK |
| | | TITNIYILNLAIADELFMLGLPFLAMQVALVHWPFGKA |
| | | ICRVVMTVDGINQFTSIFCLTVMSIDRYLAVVHPIKSA |
| | | KWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 111 | Affy6 CAR (control) | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACT |
| | | GGGTGCGCCAGGCCCCGGGACAGGGGCTGGAGTGG |
| | | ATGGGCTGGATCAAGCCTAACAACGGTCTGGCCAA |
| | | CTACGCGCAGAAGTTCCAGGGCCGCGTGACCATGA |
| | | CTCGGGACACCAGCATCTCTACCGCGTACATGGAG |
| | | CTGTCCCGCCTGCGCTCCGATGACACGGCCGTGTAC |
| | | TACTGCGCCCGCTCGGAGATCACCACCGAGTTCGACT |
| | | ATTGGGGCCAGGGCACCCTGGTGACCGTGTCGTCCg |
| | | gtggaggcggttcaggcggaggtggctctggcggtggcggatcgGACATCG |
| | | TGCTGACCCAGAGCCCTGACAGCCTGGCCGTGTCCC |
| | | TGGGCGAGCGCGCCACCATTAACTGCAAGAGCTCC |
| | | GAGAGTGTCGATAGCTACGCCAACTCCTTCATGCAC |
| | | TGGTACCAGCAGAAGCCCGGCCAGCCACCCAAGCT |
| | | GCTGATCTACAGGGCTTCCACCCGCGAGAGCGGCG |
| | | TCCCCGACAGGTTTTCAGGTTCTGGCTCCCGCACCG |
| | | ACTTCACCCTGACCATCTCTTCTCTGCAGGCCGAGG |
| | | ATGTGGCGGTGTACTACTGTCAACAGTCCAAGGAG |
| | | GACCCGCTTACCTTCGGCGGCGGCACCAAGGTGGA |
| | | GATCAAGgctagcacgaccactccggcgccgcgcccaccgactccggcccc |
| | | aactatcgcgagccagcccctgtcgctgaggccggaagcatgccgccctgccgcc |
| | | ggaggtgctgtgcatacccggggattggacttcgcatgcgacTTTTGGGTG |
| | | CTGGTGGTGGTTGGTGGAGTCCTGGCTTGCTATAGC |
| | | TTGCTAGTAACAGTGGCCTTTATTATTTTCTGGGTG |
| | | AGGAGTAAGAGGAGCAGGCTCCTGCACAGTGACTA |
| | | CATGAACATGACTCCCCGCCGCCCCGGGCCCACCC |
| | | GCAAGCATTACCAGCCCTATGCCCCACCACGCGACT |
| | | TCGCAGCCTATCGCTCCctgagagtgaagttcagcaggagcgcaga |
| | | cgcccccgcgtaccagcagggccagaaccagctctataacgagctcaatctaggac |
| | | gaagagaggagtacgatgttttggacaagagacgtggccgggaccctgagatggg |
| | | gggaaagccgagaaggaagaaccctcaggaaggcctgtacaatgaactgcagaaa |
| | | gataagatggcggaggcctacagtgagattgggatgaaaggcgagcgccggagg |
| | | ggcaaggggcacgatggcctttaccagggtctcagtacagccaccaaggacaccta |
| | | cgacgcccttcacatgcaggccctgccccctcgc |
| 112 | Affy6-I3A CAR | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACT |
| | | GGGTGCGCCAGGCCCCGGGACAGGGGCTGGAGTGG |
| | | ATGGGCTGGATCAAGCCTAACAACGGTCTGGCCAA |
| | | CTACGCGCAGAAGTTCCAGGGCCGCGTGACCATGA |
| | | CTCGGGACACCAGCATCTCTACCGCGTACATGGAG |
| | | CTGTCCCGCCTGCGCTCCGATGACACGGCCGTGTAC |
| | | TACTGCGCCCGCTCGGAGGCCACCACCGAGTTCGACT |
| | | ATTGGGGCCAGGGCACCCTGGTGACCGTGTCGTCCg |
| | | gtggaggcggttcaggcggaggtggctctggcggtggcggatcgGACATCG |
| | | TGCTGACCCAGAGCCCTGACAGCCTGGCCGTGTCCC |
| | | TGGGCGAGCGCGCCACCATTAACTGCAAGAGCTCC |
| | | GAGAGTGTCGATAGCTACGCCAACTCCTTCATGCAC |
| | | TGGTACCAGCAGAAGCCCGGCCAGCCACCCAAGCT |
| | | GCTGATCTACAGGGCTTCCACCCGCGAGAGCGGCG |
| | | TCCCCGACAGGTTTTCAGGTTCTGGCTCCCGCACCG |
| | | ACTTCACCCTGACCATCTCTTCTCTGCAGGCCGAGG |
| | | ATGTGGCGGTGTACTACTGTCAACAGTCCAAGGAG |
| | | GACCCGCTTACCTTCGGCGGCGGCACCAAGGTGGA |
| | | GATCAAGgctagcacgaccactccggcgccgcgcccaccgactccggcccc |
| | | aactatcgcgagccagcccctgtcgctgaggccggaagcatgccgccctgccgcc |
| | | ggaggtgctgtgcatacccggggattggacttcgcatgcgacTTTTGGGTG |
| | | CTGGTGGTGGTTGGTGGAGTCCTGGCTTGCTATAGC |
| | | TTGCTAGTAACAGTGGCCTTTATTATTTTCTGGGTG |
| | | AGGAGTAAGAGGAGCAGGCTCCTGCACAGTGACTA |
| | | CATGAACATGACTCCCCGCCGCCCCGGGCCCACCC |
| | | GCAAGCATTACCAGCCCTATGCCCCACCACGCGACT |
| | | TCGCAGCCTATCGCTCCctgagagtgaagttcagcaggagcgcaga |
| | | cgcccccgcgtaccagcagggccagaaccagctctataacgagctcaatctaggac |
| | | gaagagaggagtacgatgttttggacaagagacgtggccgggaccctgagatggg |
| | | gggaaagccgagaaggaagaaccctcaggaaggcctgtacaatgaactgcagaaa |
| | | gataagatggcggaggcctacagtgagattgggatgaaaggcgagcgccggagg |
| | | ggcaaggggcacgatggcctttaccagggtctcagtacagccaccaaggacaccta |
| | | cgacgcccttcacatgcaggccctgccccctcgc |
| 113 | Affy6-I3W CAR | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACT |
| | | GGGTGCGCCAGGCCCCGGGACAGGGGCTGGAGTGG |
| | | ATGGGCTGGATCAAGCCTAACAACGGTCTGGCCAA |
| | | CTACGCGCAGAAGTTCCAGGGCCGCGTGACCATGA |
| | | CTCGGGACACCAGCATCTCTACCGCGTACATGGAG |
| | | CTGTCCCGCCTGCGCTCCGATGACACGGCCGTGTAC |
| | | TACTGCGCCCGCTCGGAGTGGACCACCGAGTTCGACT |
| | | ATTGGGGCCAGGGCACCCTGGTGACCGTGTCGTCCg |
| | | gtggaggcggttcaggcggaggtggctctggcggtggcggatcgGACATCG |
| | | TGCTGACCCAGAGCCCTGACAGCCTGGCCGTGTCCC |
| | | TGGGCGAGCGCGCCACCATTAACTGCAAGAGCTCC |
| | | GAGAGTGTCGATAGCTACGCCAACTCCTTCATGCAC |
| | | TGGTACCAGCAGAAGCCCGGCCAGCCACCCAAGCT |
| | | GCTGATCTACAGGGCTTCCACCCGCGAGAGCGGCG |
| | | TCCCCGACAGGTTTTCAGGTTCTGGCTCCCGCACCG |
| | | ACTTCACCCTGACCATCTCTTCTCTGCAGGCCGAGG |
| | | ATGTGGCGGTGTACTACTGTCAACAGTCCAAGGAG |
| | | GACCCGCTTACCTTCGGCGGCGGCACCAAGGTGGA |
| | | GATCAAGgctagcacgaccactccggcgccgcgcccaccgactccggcccc |
| | | aactatcgcgagccagcccctgtcgctgaggccggaagcatgccgccctgccgcc |
| | | ggaggtgctgtgcatacccggggattggacttcgcatgcgacTTTTGGGTG |
| | | CTGGTGGTGGTTGGTGGAGTCCTGGCTTGCTATAGC |
| | | TTGCTAGTAACAGTGGCCTTTATTATTTTCTGGGTG |
| | | AGGAGTAAGAGGAGCAGGCTCCTGCACAGTGACTA |
| | | CATGAACATGACTCCCCGCCGCCCCGGGCCCACCC |
| | | GCAAGCATTACCAGCCCTATGCCCCACCACGCGACT |
| | | TCGCAGCCTATCGCTCCctgagagtgaagttcagcaggagcgcaga |
| | | cgcccccgcgtaccagcagggccagaaccagctctataacgagctcaatctaggac |
| | | gaagagaggagtacgatgttttggacaagagacgtggccgggaccctgagatggg |
| | | gggaaagccgagaaggaagaaccctcaggaaggcctgtacaatgaactgcagaaa |
| | | gataagatggcggaggcctacagtgagattgggatgaaaggcgagcgccggagg |
| | | ggcaaggggcacgatggcctttaccagggtctcagtacagccaccaaggacaccta |
| | | cgacgcccttcacatgcaggccctgccccctcgc |
| 114 | Affy6-I3F CAR | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACT |
| | | GGGTGCGCCAGGCCCCGGGACAGGGGCTGGAGTGG |
| | | ATGGGCTGGATCAAGCCTAACAACGGTCTGGCCAA |
| | | CTACGCGCAGAAGTTCCAGGGCCGCGTGACCATGA |
| | | CTCGGGACACCAGCATCTCTACCGCGTACATGGAG |
| | | CTGTCCCGCCTGCGCTCCGATGACACGGCCGTGTAC |
| | | TACTGCGCCCGCTCGGAGTTCACCACCGAGTTCGACT |
| | | ATTGGGGCCAGGGCACCCTGGTGACCGTGTCGTCCg |
| | | gtggaggcggttcaggcggaggtggctctggcggtggcggatcgGACATCG |
| | | TGCTGACCCAGAGCCCTGACAGCCTGGCCGTGTCCC |
| | | TGGGCGAGCGCGCCACCATTAACTGCAAGAGCTCC |
| | | GAGAGTGTCGATAGCTACGCCAACTCCTTCATGCAC |
| | | TGGTACCAGCAGAAGCCCGGCCAGCCACCCAAGCT |
| | | GCTGATCTACAGGGCTTCCACCCGCGAGAGCGGCG |
| | | TCCCCGACAGGTTTTCAGGTTCTGGCTCCCGCACCG |
| | | ACTTCACCCTGACCATCTCTTCTCTGCAGGCCGAGG |
| | | ATGTGGCGGTGTACTACTGTCAACAGTCCAAGGAG |
| | | GACCCGCTTACCTTCGGCGGCGGCACCAAGGTGGA |
| | | GATCAAGgctagcacgaccactccggcgccgcgcccaccgactccggcccc |
| | | aactatcgcgagccagcccctgtcgctgaggccggaagcatgccgccctgccgcc |
| | | ggaggtgctgtgcatacccggggattggacttcgcatgcgacTTTTGGGTG |
| | | CTGGTGGTGGTTGGTGGAGTCCTGGCTTGCTATAGC |
| | | TTGCTAGTAACAGTGGCCTTTATTATTTTCTGGGTG |
| | | AGGAGTAAGAGGAGCAGGCTCCTGCACAGTGACTA |
| | | CATGAACATGACTCCCCGCCGCCCCGGGCCCACCC |
| | | GCAAGCATTACCAGCCCTATGCCCCACCACGCGACT |
| | | TCGCAGCCTATCGCTCCctgagagtgaagttcagcaggagcgcaga |
| | | cgcccccgcgtaccagcagggccagaaccagctctataacgagctcaatctaggac |
| | | gaagagaggagtacgatgttttggacaagagacgtggccgggaccctgagatggg |
| | | gggaaagccgagaaggaagaaccctcaggaaggcctgtacaatgaactgcagaaa |
| | | gataagatggcggaggcctacagtgagattgggatgaaaggcgagcgccggagg |
| | | ggcaaggggcacgatggcctttaccagggtctcagtacagccaccaaggacaccta |
| | | cgacgcccttcacatgcaggccctgccccctcgc |
| 115 | Affy6-I3G CAR | CAGGTGCAGCTCGTGCAGAGCGGCGCCGAGGTGAA |
| | | GAAGCCCGGCGCTTCCGTGAAGGTGTCCTGTAAGG |
| | | CCTCTGGCTACATCTTCACCGCCTACACCATGCACT |
| | | GGGTGCGCCAGGCCCCGGGACAGGGGCTGGAGTGG |
| | | ATGGGCTGGATCAAGCCTAACAACGGTCTGGCCAA |
| | | CTACGCGCAGAAGTTCCAGGGCCGCGTGACCATGA |
| | | CTCGGGACACCAGCATCTCTACCGCGTACATGGAG |
| | | CTGTCCCGCCTGCGCTCCGATGACACGGCCGTGTAC |
| | | TACTGCGCCCGCTCGGAGGGCACCACCGAGTTCGACT |
| | | ATTGGGGCCAGGGCACCCTGGTGACCGTGTCGTCCg |
| | | gtggaggcggttcaggcggaggtggctctggcggtggcggatcgGACATCG |
| | | TGCTGACCCAGAGCCCTGACAGCCTGGCCGTGTCCC |
| | | TGGGCGAGCGCGCCACCATTAACTGCAAGAGCTCC |
| | | GAGAGTGTCGATAGCTACGCCAACTCCTTCATGCAC |
| | | TGGTACCAGCAGAAGCCCGGCCAGCCACCCAAGCT |
| | | GCTGATCTACAGGGCTTCCACCCGCGAGAGCGGCG |
| | | TCCCCGACAGGTTTTCAGGTTCTGGCTCCCGCACCG |
| | | ACTTCACCCTGACCATCTCTTCTCTGCAGGCCGAGG |
| | | ATGTGGCGGTGTACTACTGTCAACAGTCCAAGGAG |
| | | GACCCGCTTACCTTCGGCGGCGGCACCAAGGTGGA |
| | | GATCAAGgctagcacgaccactccggcgccgcgcccaccgactccggcccc |
| | | aactatcgcgagccagcccctgtcgctgaggccggaagcatgccgccctgccgcc |
| | | ggaggtgctgtgcatacccggggattggacttcgcatgcgacTTTTGGGTG |
| | | CTGGTGGTGGTTGGTGGAGTCCTGGCTTGCTATAGC |
| | | TTGCTAGTAACAGTGGCCTTTATTATTTTCTGGGTG |
| | | AGGAGTAAGAGGAGCAGGCTCCTGCACAGTGACTA |
| | | CATGAACATGACTCCCCGCCGCCCCGGGCCCACCC |
| | | GCAAGCATTACCAGCCCTATGCCCCACCACGCGACT |
| | | TCGCAGCCTATCGCTCCctgagagtgaagttcagcaggagcgcaga |
| | | cgcccccgcgtaccagcagggccagaaccagctctataacgagctcaatctaggac |
| | | gaagagaggagtacgatgttttggacaagagacgtggccgggaccctgagatggg |
| | | gggaaagccgagaaggaagaaccctcaggaaggcctgtacaatgaactgcagaaa |
| | | gataagatggcggaggcctacagtgagattgggatgaaaggcgagcgccggagg |
| | | ggcaaggggcacgatggcctttaccagggtctcagtacagccaccaaggacaccta |
| | | cgacgcccttcacatgcaggccctgccccctcgc |
| 116 | EF1α-MycT-Affy6 | ggctccggtgcccgtcagtgggcagagcgcacatcgcccacagtccccgagaagtt |
| | CAR-SSTR2 | ggggggaggggtcggcaattgaaccggtgcctagagaaggtggcgcggggtaaa |
| | (control) | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtggggggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagttt |
| | | ttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCGTGCAGAGCGGCGCCGAGGTGAAGAAGCCCGG |
| | | CGCTTCCGTGAAGGTGTCCTGTAAGGCCTCTGGCTA |
| | | CATCTTCACCGCCTACACCATGCACTGGGTGCGCCA |
| | | GGCCCCGGGACAGGGGCTGGAGTGGATGGGCTGGA |
| | | TCAAGCCTAACAACGGTCTGGCCAACTACGCGCAG |
| | | AAGTTCCAGGGCCGCGTGACCATGACTCGGGACAC |
| | | CAGCATCTCTACCGCGTACATGGAGCTGTCCCGCCT |
| | | GCGCTCCGATGACACGGCCGTGTACTACTGCGCCCG |
| | | CTCGGAGATCACCACCGAGTTCGACTATTGGGGCCAG |
| | | GGCACCCTGGTGACCGTGTCGTCCggtggaggcggttcaggc |
| | | ggaggtggctctggcggtggcggatcgGACATCGTGCTGACCCAG |
| | | AGCCCTGACAGCCTGGCCGTGTCCCTGGGCGAGCG |
| | | CGCCACCATTAACTGCAAGAGCTCCGAGAGTGTCG |
| | | ATAGCTACGCCAACTCCTTCATGCACTGGTACCAGC |
| | | AGAAGCCCGGCCAGCCACCCAAGCTGCTGATCTAC |
| | | AGGGCTTCCACCCGCGAGAGCGGCGTCCCCGACAG |
| | | GTTTTCAGGTTCTGGCTCCCGCACCGACTTCACCCT |
| | | GACCATCTCTTCTCTGCAGGCCGAGGATGTGGCGGT |
| | | GTACTACTGTCAACAGTCCAAGGAGGACCCGCTTA |
| | | CCTTCGGCGGCGGCACCAAGGTGGAGATCAAGgctag |
| | | cacgaccactccggcgccgcgcccaccgactccggccccaactatcgcgagccag |
| | | cccctgtcgctgaggccggaagcatgccgccctgccgccggaggtgctgtgcatac |
| | | ccggggattggacttcgcatgcgacTTTTGGGTGCTGGTGGTGGT |
| | | TGGTGGAGTCCTGGCTTGCTATAGCTTGCTAGTAAC |
| | | AGTGGCCTTTATTATTTTCTGGGTGAGGAGTAAGAG |
| | | GAGCAGGCTCCTGCACAGTGACTACATGAACATGA |
| | | CTCCCCGCCGCCCCGGGCCCACCCGCAAGCATTACC |
| | | AGCCCTATGCCCCACCACGCGACTTCGCAGCCTATC |
| | | GCTCCctgagagtgaagttcagcaggagcgcagacgcccccgcgtaccagca |
| | | gggccagaaccagctctataacgagctcaatctaggacgaagagaggagtacgatg |
| | | ttttggacaagagacgtggccgggaccctgagatggggggaaagccgagaaggaa |
| | | gaaccctcaggaaggcctgtacaatgaactgcagaaagataagatggcggaggcct |
| | | acagtgagattgggatgaaaggcgagcgccggaggggcaaggggcacgatggcc |
| | | tttaccagggtctcagtacagccaccaaggacacctacgacgcccttcacatgcagg |
| | | ccctgccccctcgcactagtggaagcggagctactaacttcagcctgctgaagcagg |
| | | ctggagacgtggaggagaaccctggaccttctagaatggacatggcggatgagcca |
| | | ctcaatggaagccacacatggctatccattccatttgacctcaatggctctgtggtgtca |
| | | accaacacctcaaaccagacagagccgtactatgacctgacaagcaatgcagtcctc |
| | | acattcatctattttgtggtctgcatcattgggttgtgtggcaacacacttgtcatttatgtc |
| | | atcctccgctatgccaagatgaagaccatcaccaacatttacatcctcaacctggccat |
| | | cgcagatgagctcttcatgctgggtctgcctttcttggctatgcaggtggctctggtcca |
| | | ctggccctttggcaaggccatttgccgggggtcatgactgtggatggcatcaatcagt |
| | | tcaccagcatcttctgcctgacagtcatgagcatcgaccgatacctggctgtggtccac |
| | | cccatcaagtcggccaagtggaggagaccccggacggccaagatgatcaccatgg |
| | | ctgtgtggggagtctctctgctggtcatcttgcccatcatgatatatgctgggctccgga |
| | | gcaaccagtgggggagaagcagctgcaccatcaactggccaggtgaatctggggc |
| | | ttggtacacagggttcatcatctacactttcattctggggttcctggtacccctcaccatc |
| | | atctgtctttgctacctgttcattatcatcaaggtgaagtcctctggaatccgagtgggct |
| | | cctctaagaggaagaagtctgagaagaaggtcacccgaatggtgtccatcgtggtgg |
| | | ctgtcttcatcttctgctggcttcccttctacatattcaacgtttcttccgtctccatggcca |
| | | tcagccccaccccagcccttaaaggcatgtttgactttgtggtggtcctcacctatgcta |
| | | acagctgtgccaaccctatcctatatgccttcttgtctgacaacttcaagaagagcttcc |
| | | agaatgtcctctgcttggtcaaggtgagcggcacagatgatggggagcggagtgac |
| | | agtaagcaggacaaatcccggctgaatgagaccacggagacccagaggaccctcc |
| | | tcaatggagacctccaaaccagtatctaa |
| 117 | EF1α-MycT-Affy6- | ggctccggtgcccgtcagtgggcagagcgcacatcgcccacagtccccgagaagtt |
| | I3A CAR-SSTR2 | ggggggaggggtcggcaattgaaccggtgcctagagaaggtggcgcggggtaaa |
| | | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtggggggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagttt |
| | | ttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCGTGCAGAGCGGCGCCGAGGTGAAGAAGCCCGG |
| | | CGCTTCCGTGAAGGTGTCCTGTAAGGCCTCTGGCTA |
| | | CATCTTCACCGCCTACACCATGCACTGGGTGCGCCA |
| | | GGCCCCGGGACAGGGGCTGGAGTGGATGGGCTGGA |
| | | TCAAGCCTAACAACGGTCTGGCCAACTACGCGCAG |
| | | AAGTTCCAGGGCCGCGTGACCATGACTCGGGACAC |
| | | CAGCATCTCTACCGCGTACATGGAGCTGTCCCGCCT |
| | | GCGCTCCGATGACACGGCCGTGTACTACTGCGCCCG |
| | | CTCGGAGGCCACCACCGAGTTCGACTATTGGGGCCAG |
| | | GGCACCCTGGTGACCGTGTCGTCCggtggaggcggttcaggc |
| | | ggaggtggctctggcggtggcggatcgGACATCGTGCTGACCCAG |
| | | AGCCCTGACAGCCTGGCCGTGTCCCTGGGCGAGCG |
| | | CGCCACCATTAACTGCAAGAGCTCCGAGAGTGTCG |
| | | ATAGCTACGCCAACTCCTTCATGCACTGGTACCAGC |
| | | AGAAGCCCGGCCAGCCACCCAAGCTGCTGATCTAC |
| | | AGGGCTTCCACCCGCGAGAGCGGCGTCCCCGACAG |
| | | GTTTTCAGGTTCTGGCTCCCGCACCGACTTCACCCT |
| | | GACCATCTCTTCTCTGCAGGCCGAGGATGTGGCGGT |
| | | GTACTACTGTCAACAGTCCAAGGAGGACCCGCTTA |
| | | CCTTCGGCGGCGGCACCAAGGTGGAGATCAAGgctag |
| | | cacgaccactccggcgccgcgcccaccgactccggccccaactatcgcgagccag |
| | | cccctgtcgctgaggccggaagcatgccgccctgccgccggaggtgctgtgcatac |
| | | ccggggattggacttcgcatgcgacTTTTGGGTGCTGGTGGTGGT |
| | | TGGTGGAGTCCTGGCTTGCTATAGCTTGCTAGTAAC |
| | | AGTGGCCTTTATTATTTTCTGGGTGAGGAGTAAGAG |
| | | GAGCAGGCTCCTGCACAGTGACTACATGAACATGA |
| | | CTCCCCGCCGCCCCGGGCCCACCCGCAAGCATTACC |
| | | AGCCCTATGCCCCACCACGCGACTTCGCAGCCTATC |
| | | GCTCCctgagagtgaagttcagcaggagcgcagacgcccccgcgtaccagca |
| | | gggccagaaccagctctataacgagctcaatctaggacgaagagaggagtacgatg |
| | | ttttggacaagagacgtggccgggaccctgagatggggggaaagccgagaaggaa |
| | | gaaccctcaggaaggcctgtacaatgaactgcagaaagataagatggcggaggcct |
| | | acagtgagattgggatgaaaggcgagcgccggaggggcaaggggcacgatggcc |
| | | tttaccagggtctcagtacagccaccaaggacacctacgacgcccttcacatgcagg |
| | | ccctgccccctcgcactagtggaagcggagctactaacttcagcctgctgaagcagg |
| | | ctggagacgtggaggagaaccctggaccttctagaatggacatggcggatgagcca |
| | | ctcaatggaagccacacatggctatccattccatttgacctcaatggctctgtggtgtca |
| | | accaacacctcaaaccagacagagccgtactatgacctgacaagcaatgcagtcctc |
| | | acattcatctattttgtggtctgcatcattgggttgtgtggcaacacacttgtcatttatgtc |
| | | atcctccgctatgccaagatgaagaccatcaccaacatttacatcctcaacctggccat |
| | | cgcagatgagctcttcatgctgggtctgcctttcttggctatgcaggtggctctggtcca |
| | | ctggccctttggcaaggccatttgccgggtggtcatgactgtggatggcatcaatcagt |
| | | tcaccagcatcttctgcctgacagtcatgagcatcgaccgatacctggctgtggtccac |
| | | cccatcaagtcggccaagtggaggagaccccggacggccaagatgatcaccatgg |
| | | ctgtgtggggagtctctctgctggtcatcttgcccatcatgatatatgctgggctccgga |
| | | gcaaccagtgggggagaagcagctgcaccatcaactggccaggtgaatctggggc |
| | | ttggtacacagggttcatcatctacactttcattctggggttcctggtacccctcaccatc |
| | | atctgtctttgctacctgttcattatcatcaaggtgaagtcctctggaatccgagtgggct |
| | | cctctaagaggaagaagtctgagaagaaggtcacccgaatggtgtccatcgtggtgg |
| | | ctgtcttcatcttctgctggcttcccttctacatattcaacgtttcttccgtctccatggcca |
| | | tcagccccaccccagcccttaaaggcatgtttgactttgtggtggtcctcacctatgcta |
| | | acagctgtgccaaccctatcctatatgccttcttgtctgacaacttcaagaagagcttcc |
| | | agaatgtcctctgcttggtcaaggtgagcggcacagatgatggggagcggagtgac |
| | | agtaagcaggacaaatcccggctgaatgagaccacggagacccagaggaccctcc |
| | | tcaatggagacctccaaaccagtatctaa |
| 118 | EF1α-MycT-Affy6- | ggctccggtgcccgtcagtgggcagagcgcacatcgcccacagtccccgagaagtt |
| | I3W CAR-SSTR2 | ggggggaggggtcggcaattgaaccggtgcctagagaaggtggcgcggggtaaa |
| | | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtgggtgggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagttt |
| | | ttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCGTGCAGAGCGGCGCCGAGGTGAAGAAGCCCGG |
| | | CGCTTCCGTGAAGGTGTCCTGTAAGGCCTCTGGCTA |
| | | CATCTTCACCGCCTACACCATGCACTGGGTGCGCCA |
| | | GGCCCCGGGACAGGGGCTGGAGTGGATGGGCTGGA |
| | | TCAAGCCTAACAACGGTCTGGCCAACTACGCGCAG |
| | | AAGTTCCAGGGCCGCGTGACCATGACTCGGGACAC |
| | | CAGCATCTCTACCGCGTACATGGAGCTGTCCCGCCT |
| | | GCGCTCCGATGACACGGCCGTGTACTACTGCGCCCG |
| | | CTCGGAGTGGACCACCGAGTTCGACTATTGGGGCCAG |
| | | GGCACCCTGGTGACCGTGTCGTCCggtggaggcggttcaggc |
| | | ggaggtggctctggcggtggcggatcgGACATCGTGCTGACCCAG |
| | | AGCCCTGACAGCCTGGCCGTGTCCCTGGGCGAGCG |
| | | CGCCACCATTAACTGCAAGAGCTCCGAGAGTGTCG |
| | | ATAGCTACGCCAACTCCTTCATGCACTGGTACCAGC |
| | | AGAAGCCCGGCCAGCCACCCAAGCTGCTGATCTAC |
| | | AGGGCTTCCACCCGCGAGAGCGGCGTCCCCGACAG |
| | | GTTTTCAGGTTCTGGCTCCCGCACCGACTTCACCCT |
| | | GACCATCTCTTCTCTGCAGGCCGAGGATGTGGCGGT |
| | | GTACTACTGTCAACAGTCCAAGGAGGACCCGCTTA |
| | | CCTTCGGCGGCGGCACCAAGGTGGAGATCAAGgctag |
| | | cacgaccactccggcgccgcgcccaccgactccggccccaactatcgcgagccag |
| | | cccctgtcgctgaggccggaagcatgccgccctgccgccggaggtgctgtgcatac |
| | | ccggggattggacttcgcatgcgacTTTTGGGTGCTGGTGGTGGT |
| | | TGGTGGAGTCCTGGCTTGCTATAGCTTGCTAGTAAC |
| | | AGTGGCCTTTATTATTTTCTGGGTGAGGAGTAAGAG |
| | | GAGCAGGCTCCTGCACAGTGACTACATGAACATGA |
| | | CTCCCCGCCGCCCCGGGCCCACCCGCAAGCATTACC |
| | | AGCCCTATGCCCCACCACGCGACTTCGCAGCCTATC |
| | | GCTCCctgagagtgaagttcagcaggagcgcagacgcccccgcgtaccagca |
| | | gggccagaaccagctctataacgagctcaatctaggacgaagagaggagtacgatg |
| | | ttttggacaagagacgtggccgggaccctgagatggggggaaagccgagaaggaa |
| | | gaaccctcaggaaggcctgtacaatgaactgcagaaagataagatggcggaggcct |
| | | acagtgagattgggatgaaaggcgagcgccggaggggcaaggggcacgatggcc |
| | | tttaccagggtctcagtacagccaccaaggacacctacgacgcccttcacatgcagg |
| | | ccctgccccctcgcactagtggaagcggagctactaacttcagcctgctgaagcagg |
| | | ctggagacgtggaggagaaccctggaccttctagaatggacatggcggatgagcca |
| | | ctcaatggaagccacacatggctatccattccatttgacctcaatggctctgtggtgtca |
| | | accaacacctcaaaccagacagagccgtactatgacctgacaagcaatgcagtcctc |
| | | acattcatctattttgtggtctgcatcattgggttgtgtggcaacacacttgtcatttatgtc |
| | | atcctccgctatgccaagatgaagaccatcaccaacatttacatcctcaacctggccat |
| | | cgcagatgagctcttcatgctgggtctgcctttcttggctatgcaggtggctctggtcca |
| | | ctggccctttggcaaggccatttgccgggtggtcatgactgtggatggcatcaatcagt |
| | | tcaccagcatcttctgcctgacagtcatgagcatcgaccgatacctggctgtggtccac |
| | | cccatcaagtcggccaagtggaggagaccccggacggccaagatgatcaccatgg |
| | | ctgtgtggggagtctctctgctggtcatcttgcccatcatgatatatgctgggctccgga |
| | | gcaaccagtgggggagaagcagctgcaccatcaactggccaggtgaatctggggc |
| | | ttggtacacagggttcatcatctacactttcattctggggttcctggtacccctcaccatc |
| | | atctgtctttgctacctgttcattatcatcaaggtgaagtcctctggaatccgagtgggct |
| | | cctctaagaggaagaagtctgagaagaaggtcacccgaatggtgtccatcgtggtgg |
| | | ctgtcttcatcttctgctggcttcccttctacatattcaacgtttcttccgtctccatggcca |
| | | tcagccccaccccagcccttaaaggcatgtttgactttgtggtggtcctcacctatgcta |
| | | acagctgtgccaaccctatcctatatgccttcttgtctgacaacttcaagaagagcttcc |
| | | agaatgtcctctgcttggtcaaggtgagcggcacagatgatggggagcggagtgac |
| | | agtaagcaggacaaatcccggctgaatgagaccacggagacccagaggaccctcc |
| | | tcaatggagacctccaaaccagtatctaa |
| 119 | EF1α-MycT-Affy6- | ggctccggtgcccgtcagtgggcagagcgcacatcgcccacagtccccgagaagtt |
| | I3F CAR-SSTR2 | ggggggaggggtcggcaattgaaccggtgcctagagaaggtggcgcggggtaaa |
| | | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtgggtgggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagttt |
| | | ttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCGTGCAGAGCGGCGCCGAGGTGAAGAAGCCCGG |
| | | CGCTTCCGTGAAGGTGTCCTGTAAGGCCTCTGGCTA |
| | | CATCTTCACCGCCTACACCATGCACTGGGTGCGCCA |
| | | GGCCCCGGGACAGGGGCTGGAGTGGATGGGCTGGA |
| | | TCAAGCCTAACAACGGTCTGGCCAACTACGCGCAG |
| | | AAGTTCCAGGGCCGCGTGACCATGACTCGGGACAC |
| | | CAGCATCTCTACCGCGTACATGGAGCTGTCCCGCCT |
| | | GCGCTCCGATGACACGGCCGTGTACTACTGCGCCCG |
| | | CTCGGAGTTCACCACCGAGTTCGACTATTGGGGCCAG |
| | | GGCACCCTGGTGACCGTGTCGTCCggtggaggcggttcaggc |
| | | ggaggtggctctggcggtggcggatcgGACATCGTGCTGACCCAG |
| | | AGCCCTGACAGCCTGGCCGTGTCCCTGGGCGAGCG |
| | | CGCCACCATTAACTGCAAGAGCTCCGAGAGTGTCG |
| | | ATAGCTACGCCAACTCCTTCATGCACTGGTACCAGC |
| | | AGAAGCCCGGCCAGCCACCCAAGCTGCTGATCTAC |
| | | AGGGCTTCCACCCGCGAGAGCGGCGTCCCCGACAG |
| | | GTTTTCAGGTTCTGGCTCCCGCACCGACTTCACCCT |
| | | GACCATCTCTTCTCTGCAGGCCGAGGATGTGGCGGT |
| | | GTACTACTGTCAACAGTCCAAGGAGGACCCGCTTA |
| | | CCTTCGGCGGCGGCACCAAGGTGGAGATCAAGgctag |
| | | cacgaccactccggcgccgcgcccaccgactccggccccaactatcgcgagccag |
| | | cccctgtcgctgaggccggaagcatgccgccctgccgccggaggtgctgtgcatac |
| | | ccggggattggacttcgcatgcgacTTTTGGGTGCTGGTGGTGGT |
| | | TGGTGGAGTCCTGGCTTGCTATAGCTTGCTAGTAAC |
| | | AGTGGCCTTTATTATTTTCTGGGTGAGGAGTAAGAG |
| | | GAGCAGGCTCCTGCACAGTGACTACATGAACATGA |
| | | CTCCCCGCCGCCCCGGGCCCACCCGCAAGCATTACC |
| | | AGCCCTATGCCCCACCACGCGACTTCGCAGCCTATC |
| | | GCTCCctgagagtgaagttcagcaggagcgcagacgcccccgcgtaccagca |
| | | gggccagaaccagctctataacgagctcaatctaggacgaagagaggagtacgatg |
| | | ttttggacaagagacgtggccgggaccctgagatggggggaaagccgagaaggaa |
| | | gaaccctcaggaaggcctgtacaatgaactgcagaaagataagatggcggaggcct |
| | | acagtgagattgggatgaaaggcgagcgccggaggggcaaggggcacgatggcc |
| | | tttaccagggtctcagtacagccaccaaggacacctacgacgcccttcacatgcagg |
| | | ccctgccccctcgcactagtggaagcggagctactaacttcagcctgctgaagcagg |
| | | ctggagacgtggaggagaaccctggaccttctagaatggacatggcggatgagcca |
| | | ctcaatggaagccacacatggctatccattccatttgacctcaatggctctgtggtgtca |
| | | accaacacctcaaaccagacagagccgtactatgacctgacaagcaatgcagtcctc |
| | | acattcatctattttgtggtctgcatcattgggttgtgtggcaacacacttgtcatttatgtc |
| | | atcctccgctatgccaagatgaagaccatcaccaacatttacatcctcaacctggccat |
| | | cgcagatgagctcttcatgctgggtctgcctttcttggctatgcaggtggctctggtcca |
| | | ctggccctttggcaaggccatttgccgggtggtcatgactgtggatggcatcaatcagt |
| | | tcaccagcatcttctgcctgacagtcatgagcatcgaccgatacctggctgtggtccac |
| | | cccatcaagtcggccaagtggaggagaccccggacggccaagatgatcaccatgg |
| | | ctgtgtggggagtctctctgctggtcatcttgcccatcatgatatatgctgggctccgga |
| | | gcaaccagtgggggagaagcagctgcaccatcaactggccaggtgaatctggggc |
| | | ttggtacacagggttcatcatctacactttcattctggggttcctggtacccctcaccatc |
| | | atctgtctttgctacctgttcattatcatcaaggtgaagtcctctggaatccgagtgggct |
| | | cctctaagaggaagaagtctgagaagaaggtcacccgaatggtgtccatcgtggtgg |
| | | ctgtcttcatcttctgctggcttcccttctacatattcaacgtttcttccgtctccatggcca |
| | | tcagccccaccccagcccttaaaggcatgtttgactttgtggtggtcctcacctatgcta |
| | | acagctgtgccaaccctatcctatatgccttcttgtctgacaacttcaagaagagcttcc |
| | | agaatgtcctctgcttggtcaaggtgagcggcacagatgatggggagcggagtgac |
| | | agtaagcaggacaaatcccggctgaatgagaccacggagacccagaggaccctcc |
| | | tcaatggagacctccaaaccagtatctaa |
| 120 | EF1α-MycT-Affy6- | ggctccggtgcccgtcagtgggcagagcgcacatcgcccacagtccccgagaagtt |
| | I3G CAR-SSTR2 | ggggggaggggtcggcaattgaaccggtgcctagagaaggtggcgcggggtaaa |
| | | ctgggaaagtgatgtcgtgtactggctccgcctttttcccgagggtgggggagaacc |
| | | gtatataagtgcagtagtcgccgtgaacgttctttttcgcaacgggtttgccgccagaa |
| | | cacaggtaagtgccgtgtgtggttcccgcgggcctggcctctttacgggttatggccc |
| | | ttgcgtgccttgaattacttccacctggctgcagtacgtgattcttgatcccgagcttcgg |
| | | gttggaagtggggggagagttcgaggccttgcgcttaaggagccccttcgcctcgt |
| | | gcttgagttgaggcctggcctgggcgctggggccgccgcgtgcgaatctggtggca |
| | | ccttcgcgcctgtctcgctgctttcgataagtctctagccatttaaaatttttgatgacctg |
| | | ctgcgacgctttttttctggcaagatagtcttgtaaatgcgggccaagatctgcacactg |
| | | gtatttcggtttttggggccgcgggcggcgacggggcccgtgcgtcccagcgcacat |
| | | gttcggcgaggcggggcctgcgagcgcggccaccgagaatcggacgggggtagt |
| | | ctcaagctggccggcctgctctggtgcctggtctcgcgccgccgtgtatcgccccgc |
| | | cctgggcggcaaggctggcccggtcggcaccagttgcgtgagcggaaagatggcc |
| | | gcttcccggccctgctgcagggagctcaaaatggaggacgcggcgctcgggagag |
| | | cgggcgggtgagtcacccacacaaaggaaaagggcctttccgtcctcagccgtcgc |
| | | ttcatgtgactccacggagtaccgggcgccgtccaggcacctcgattagttctcgagc |
| | | ttttggagtacgtcgtctttaggttggggggaggggttttatgcgatggagtttccccac |
| | | actgagtgggtggagactgaagttaggccagcttggcacttgatgtaattctccttgga |
| | | atttgccctttttgagtttggatcttggttcattctcaagcctcagacagtggttcaaagttt |
| | | ttttcttccatttcaggtgtcgtgacaagtttgtacaaaaaagcaggctgccaccatggc |
| | | cttaccagtgaccgccttgctcctgccgctggccttgctgctccacgccgccaggccg |
| | | ggatccgaacaaaaactcatctcagaagaggatctgggatcgCAGGTGCAG |
| | | CTCGTGCAGAGCGGCGCCGAGGTGAAGAAGCCCGG |
| | | CGCTTCCGTGAAGGTGTCCTGTAAGGCCTCTGGCTA |
| | | CATCTTCACCGCCTACACCATGCACTGGGTGCGCCA |
| | | GGCCCCGGGACAGGGGCTGGAGTGGATGGGCTGGA |
| | | TCAAGCCTAACAACGGTCTGGCCAACTACGCGCAG |
| | | AAGTTCCAGGGCCGCGTGACCATGACTCGGGACAC |
| | | CAGCATCTCTACCGCGTACATGGAGCTGTCCCGCCT |
| | | GCGCTCCGATGACACGGCCGTGTACTACTGCGCCCG |
| | | CTCGGAGGGCACCACCGAGTTCGACTATTGGGGCCAG |
| | | GGCACCCTGGTGACCGTGTCGTCCggtggaggcggttcaggc |
| | | ggaggtggctctggcggtggcggatcgGACATCGTGCTGACCCAG |
| | | AGCCCTGACAGCCTGGCCGTGTCCCTGGGCGAGCG |
| | | CGCCACCATTAACTGCAAGAGCTCCGAGAGTGTCG |
| | | ATAGCTACGCCAACTCCTTCATGCACTGGTACCAGC |
| | | AGAAGCCCGGCCAGCCACCCAAGCTGCTGATCTAC |
| | | AGGGCTTCCACCCGCGAGAGCGGCGTCCCCGACAG |
| | | GTTTTCAGGTTCTGGCTCCCGCACCGACTTCACCCT |
| | | GACCATCTCTTCTCTGCAGGCCGAGGATGTGGCGGT |
| | | GTACTACTGTCAACAGTCCAAGGAGGACCCGCTTA |
| | | CCTTCGGCGGCGGCACCAAGGTGGAGATCAAGgctag |
| | | cacgaccactccggcgccgcgcccaccgactccggccccaactatcgcgagccag |
| | | cccctgtcgctgaggccggaagcatgccgccctgccgccggaggtgctgtgcatac |
| | | ccggggattggacttcgcatgcgacTTTTGGGTGCTGGTGGTGGT |
| | | TGGTGGAGTCCTGGCTTGCTATAGCTTGCTAGTAAC |
| | | AGTGGCCTTTATTATTTTCTGGGTGAGGAGTAAGAG |
| | | GAGCAGGCTCCTGCACAGTGACTACATGAACATGA |
| | | CTCCCCGCCGCCCCGGGCCCACCCGCAAGCATTACC |
| | | AGCCCTATGCCCCACCACGCGACTTCGCAGCCTATC |
| | | GCTCCctgagagtgaagttcagcaggagcgcagacgcccccgcgtaccagca |
| | | gggccagaaccagctctataacgagctcaatctaggacgaagagaggagtacgatg |
| | | ttttggacaagagacgtggccgggaccctgagatggggggaaagccgagaaggaa |
| | | gaaccctcaggaaggcctgtacaatgaactgcagaaagataagatggcggaggcct |
| | | acagtgagattgggatgaaaggcgagcgccggaggggcaaggggcacgatggcc |
| | | tttaccagggtctcagtacagccaccaaggacacctacgacgcccttcacatgcagg |
| | | ccctgccccctcgcactagtggaagcggagctactaacttcagcctgctgaagcagg |
| | | ctggagacgtggaggagaaccctggaccttctagaatggacatggcggatgagcca |
| | | ctcaatggaagccacacatggctatccattccatttgacctcaatggctctgtggtgtca |
| | | accaacacctcaaaccagacagagccgtactatgacctgacaagcaatgcagtcctc |
| | | acattcatctattttgtggtctgcatcattgggttgtgtggcaacacacttgtcatttatgtc |
| | | atcctccgctatgccaagatgaagaccatcaccaacatttacatcctcaacctggccat |
| | | cgcagatgagctcttcatgctgggtctgcctttcttggctatgcaggtggctctggtcca |
| | | ctggccctttggcaaggccatttgccgggtggtcatgactgtggatggcatcaatcagt |
| | | tcaccagcatcttctgcctgacagtcatgagcatcgaccgatacctggctgtggtccac |
| | | cccatcaagtcggccaagtggaggagaccccggacggccaagatgatcaccatgg |
| | | ctgtgtggggagtctctctgctggtcatcttgcccatcatgatatatgctgggctccgga |
| | | gcaaccagtgggggagaagcagctgcaccatcaactggccaggtgaatctggggc |
| | | ttggtacacagggttcatcatctacactttcattctggggttcctggtacccctcaccatc |
| | | atctgtctttgctacctgttcattatcatcaaggtgaagtcctctggaatccgagtgggct |
| | | cctctaagaggaagaagtctgagaagaaggtcacccgaatggtgtccatcgtggtgg |
| | | ctgtcttcatcttctgctggcttcccttctacatattcaacgtttcttccgtctccatggcca |
| | | tcagccccaccccagcccttaaaggcatgtttgactttgtggtggtcctcacctatgcta |
| | | acagctgtgccaaccctatcctatatgccttcttgtctgacaacttcaagaagagcttcc |
| | | agaatgtcctctgcttggtcaaggtgagcggcacagatgatggggagcggagtgac |
| | | agtaagcaggacaaatcccggctgaatgagaccacggagacccagaggaccctcc |
| | | tcaatggagacctccaaaccagtatctaa |
| 121 | (GGGGS)x3 linker | GGGGSGGGGSGGGGS |
| 122 | 4-1BB costimulatory | KRGRKKLLYIFKQPFMRPVQTTQEEDGCSCRFPEEEE |
| | domain | GGCEL |
| 123 | CD28 costimulatory | RSKRSRLLHSDYMNMTPRRPGPTRKHYQPYAPPRDFA |
| | domain | AYRS |
| 124 | OX-40 costimulatory | ALYLLRRDQRLPPDAHKPPGGGSFRTPIQEEQADAHS |
| | domain | TLAKI |
| 125 | CD3 zeta signaling | RVKFSRSADAPAYKQGQNQLYNELNLGRREEYDVLD |
| | domain | KRRGRDPEMGGKPRRKNPQEGLYNELQKDKMAEAY |
| | | SEIGMKGERRRGKGHDGLYQGLSTATKDTYDALHMQ |
| | | ALPPR |
| 126 | CD8 alpha hinge | TTTPAPRPPTPAPTIASQPLSLRPEACRPAAGGAVHTR |
| | | GLDFACD |
| 127 | CD28 hinge | RAAAIEVMYPPPYLDNEKSNGTIIHVKGKHLCPSPLFP |
| | | GPSKPKDPK |
| 128 | IgG4 hinge | SKYGPPCPSCP |
| 129 | CD8 alpha | IYIWAPLAGTCGVLLLSLVITLYC |
| | transmembrane | |
| | domain | |
| 130 | CD28 | FWVLVVVGGVLACYSLLVTVAFII |
| | transmembrane | |
| | domain | |
| 131 | ICOS | FWLPIGCAAFVVVCILGCILICWL |
| | transmembrane | |
| | domain | |
| 132 | GITR | LGWLTVVLLAVAACVLLLTSAQLGL |
| | transmembrane | |
| | domain | |
| 133 | αL subunit of LFA-1 | YNLDVRGARSFSPPRAGRHFGYRVLQVGNGVIVGAP |
| | | GEGNSTGSLYQCQSGTGHCLPVTLRGSNYTSKYLGM |
| | | TLATDPTDGSILACDPGLSRTCDQNTYLSGLCYLFRQN |
| | | LQGPMLQGRPGFQECIKGNVDLVFLFDGSMSLQPDEF |
| | | QKILDFMKDVMKKLSNTSYQFAAVQFSTSYKTEFDFS |
| | | DYVKWKDPDALLKHVKHMLLLTNTFGAINYVATEVF |
| | | REELGARPDATKVLIIITDGEATDSGNIDAAKDIIR YIIG |
| | | IGKHFQTKESQETLHKFASKPASEFVKILDTFEKLKDL |
| | | FTELQKKIYVIEGTSKQDLTSFNMELSSSGISADLSRGH |
| | | AVVGAVGAKDWAGGFLDLKADLQDDTFIGNEPLTPE |
| | | VRAGYLGYTVTWLPSRQKTSLLASGAPRYQHMGRVL |
| | | LFQEPQGGGHWSQVQTIHGTQIGSYFGGELCGVDVD |
| | | QDGETELLLIGAPLFYGEQRGGRVFIYQRRQLGFEEVS |
| | | ELQGDPGYPLGRFGEAITALTDINGDGLVDVAVGAPL |
| | | EEQGAVYIFNGRHGGLSPQPSQRIEGTQVLSGIQWFGR |
| | | SIHGVKDLEGDGLADVAVGAESQMIVLSSRPVVDMV |
| | | TLMSFSPAEIPVHEVECSYSTSNKMKEGVNITICFQIKS |
| | | LYPQFQGRLVANLTYTLQLDGHRTRRRGLFPGGRHEL |
| | | RRNIAVTTSMSCTDFSFHFPVCVQDLISPINVSLNFSLW |
| | | EEEGTPRDQRAQGKDIPPILRPSLHSETWEIPFEKNCGE |
| | | DKKCEANLRVSFSPARSRALRLTAFASLSVELSLSNLE |
| | | EDAYWVQLDLHFPPGLSFRKVEMLKPHSQIPVSCEEL |
| | | PEESRLLSRALSCNVSSPIFKAGHSVALQMMENTLVNS |
| | | SWGDSVELHANVTCNNEDSDLLEDNSATTIIPILYPINI |
| | | LIQDQEDSTLYVSFTPKGPKIHQVKHMYQVRIQPSIHD |
| | | HNIPTLEAVVGVPQPPSEGPITHQWSVQMEPPVPCHYE |
| | | DLERLPDAAEPCLPGALFRCPVVFRQEILVQVIGTLEL |
| | | VGEIEASSMFSLCSSLSISFNSSKHFHLYGSNASLAQVV |
| | | MKVDVVYEKQMLYLYVLSGIGGLLLLLLIFIVLYKVG |
| | | FFKRNLKEKMEAGRGVPNGIPAEDSEQLASGQEAGDP |
| | | GCLKPLHEKDSESGGGKD |
| 134 | I domain of αL | VDLVFLFDGSMSLQPDEFQKILDFMKDVMKKLSNTSY |
| | subunit of LFA-1 | QFAAVQFSTSYKTEFDFSDYVKWKDPDALLKHVKHM |
| | | LLLTNTFGAINYVATEVFREELGARPDATKVLIIITDGE |
| | | ATDSGNIDAAKDIIRYIIGIGKHFQTKESQETLHKFASK |
| | | PASEFVKILDTFEKLKDLFTELQKKIYVIE |
| 135 | SSTR2 (aa1-381) | MDMADEPLNGSHTWLSIPFDLNGSVVSTNTSNQTEPY |
| | | YDLTSNAVLTFIYFVVCIIGLCGNTLVIYVILRYAKMK |
| | | TITNIYILNLAIADELFMLGLPFLAMQVALVHWPFGKA |
| | | ICRVVMTVDGINQFTSIFCLTVMSIDRYLAVVHPIKSA |
| | | KWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAFLSDNFKKSFQNVLCLVKVSGTDDGER |
| | | SDSKQDKSRLNETTETQRTLLNGDLQTSI |
| 136 | SSTR2 (aa 1-314) | MDMADEPLNGSHTWLSIPFDLNGSVVSTNTSNQTEPY |
| | | YDLTSNAVLTFIYFVVCIIGLCGNTLVIYVILRYAKMK |
| | | TITNIYILNLAIADELFMLGLPFLAMQVALVHWPFGKA |
| | | ICRVVMTVDGINQFTSIFCLTVMSIDRYLAVVHPIKSA |
| | | KWRRPRTAKMITMAVWGVSLLVILPIMIYAGLRSNQ |
| | | WGRSSCTINWPGESGAWYTGFIIYTFILGFLVPLTIICL |
| | | CYLFIIIKVKSSGIRVGSSKRKKSEKKVTRMVSIVVAVF |
| | | IFCWLPFYIFNVSSVSMAISPTPALKGMFDFVVVLTYA |
| | | NSCANPILYAF |
| 137 | SSTR2 (aa 1-381) | ATGGACATGGCGGATGAGCCACTCAATGGAAGCCA |
| | nucleotide sequence | CACATGGCTATCCATTCCATTTGACCTCAATGGCTC |
| | | TGTGGTGTCAACCAACACCTCAAACCAGACAGAGC |
| | | CGTACTATGACCTGACAAGCAATGCAGTCCTCACAT |
| | | TCATCTATTTTGTGGTCTGCATCATTGGGTTGTGTGG |
| | | CAACACACTTGTCATTTATGTCATCCTCCGCTATGC |
| | | CAAGATGAAGACCATCACCAACATTTACATCCTCA |
| | | ACCTGGCCATCGCAGATGAGCTCTTCATGCTGGGTC |
| | | TGCCTTTCTTGGCTATGCAGGTGGCTCTGGTCCACT |
| | | GGCCCTTTGGCAAGGCCATTTGCCGGGTGGTCATGA |
| | | CTGTGGATGGCATCAATCAGTTCACCAGCATCTTCT |
| | | GCCTGACAGTCATGAGCATCGACCGATACCTGGCT |
| | | GTGGTCCACCCCATCAAGTCGGCCAAGTGGAGGAG |
| | | ACCCCGGACGGCCAAGATGATCACCATGGCTGTGT |
| | | GGGGAGTCTCTCTGCTGGTCATCTTGCCCATCATGA |
| | | TATATGCTGGGCTCCGGAGCAACCAGTGGGGGAGA |
| | | AGCAGCTGCACCATCAACTGGCCAGGTGAATCTGG |
| | | GGCTTGGTACACAGGGTTCATCATCTACACTTTCAT |
| | | TCTGGGGTTCCTGGTACCCCTCACCATCATCTGTCTT |
| | | TGCTACCTGTTCATTATCATCAAGGTGAAGTCCTCT |
| | | GGAATCCGAGTGGGCTCCTCTAAGAGGAAGAAGTC |
| | | TGAGAAGAAGGTCACCCGAATGGTGTCCATCGTGG |
| | | TGGCTGTCTTCATCTTCTGCTGGCTTCCCTTCTACAT |
| | | ATTCAACGTTTCTTCCGTCTCCATGGCCATCAGCCC |
| | | CACCCCAGCCCTTAAAGGCATGTTTGACTTTGTGGT |
| | | GGTCCTCACCTATGCTAACAGCTGTGCCAACCCTAT |
| | | CCTATATGCCTTCTTGTCTGACAACTTCAAGAAGAG |
| | | CTTCCAGAATGTCCTCTGCTTGGTCAAGGTGAGCGG |
| | | CACAGATGATGGGGAGCGGAGTGACAGTAAGCAGG |
| | | ACAAATCCCGGCTGAATGAGACCACGGAGACCCAG |
| | | AGGACCCTCCTCAATGGAGACCTCCAAACCAGTAT |
| | | CTAA |
| 138 | SSTR2 (aa1-314) | ATGGACATGGCGGATGAGCCACTCAATGGAAGCCA |
| | nucleotide sequence | CACATGGCTATCCATTCCATTTGACCTCAATGGCTC |
| | | TGTGGTGTCAACCAACACCTCAAACCAGACAGAGC |
| | | CGTACTATGACCTGACAAGCAATGCAGTCCTCACAT |
| | | TCATCTATTTTGTGGTCTGCATCATTGGGTTGTGTGG |
| | | CAACACACTTGTCATTTATGTCATCCTCCGCTATGC |
| | | CAAGATGAAGACCATCACCAACATTTACATCCTCA |
| | | ACCTGGCCATCGCAGATGAGCTCTTCATGCTGGGTC |
| | | TGCCTTTCTTGGCTATGCAGGTGGCTCTGGTCCACT |
| | | GGCCCTTTGGCAAGGCCATTTGCCGGGTGGTCATGA |
| | | CTGTGGATGGCATCAATCAGTTCACCAGCATCTTCT |
| | | GCCTGACAGTCATGAGCATCGACCGATACCTGGCT |
| | | GTGGTCCACCCCATCAAGTCGGCCAAGTGGAGGAG |
| | | ACCCCGGACGGCCAAGATGATCACCATGGCTGTGT |
| | | GGGGAGTCTCTCTGCTGGTCATCTTGCCCATCATGA |
| | | TATATGCTGGGCTCCGGAGCAACCAGTGGGGGAGA |
| | | AGCAGCTGCACCATCAACTGGCCAGGTGAATCTGG |
| | | GGCTTGGTACACAGGGTTCATCATCTACACTTTCAT |
| | | TCTGGGGTTCCTGGTACCCCTCACCATCATCTGTCTT |
| | | TGCTACCTGTTCATTATCATCAAGGTGAAGTCCTCT |
| | | GGAATCCGAGTGGGCTCCTCTAAGAGGAAGAAGTC |
| | | TGAGAAGAAGGTCACCCGAATGGTGTCCATCGTGG |
| | | TGGCTGTCTTCATCTTCTGCTGGCTTCCCTTCTACAT |
| | | ATTCAACGTTTCTTCCGTCTCCATGGCCATCAGCCC |
| | | CACCCCAGCCCTTAAAGGCATGTTTGACTTTGTGGT |
| | | GGTCCTCACCTATGCTAACAGCTGTGCCAACCCTAT |
| | | CCTATAT |
| 139 | P2A peptide | GSGATNFSLLKQAGDVEENPGP |
| 140 | E2A peptide | GSGQCTNYALLKLAGDVESNPGP |
| 141 | T2A peptide | GSGEGRGSLLTCGDVEENPGP |
| 142 | F2A peptide | GSGVKQTLNFDLLKLAGDVESNPGP |
| 143 | Linker | (GGGS)n |
| 144 | AB_001 primer | CTGTGCCACCgcgGGCTCCTACGTGAGC |
| 145 | AB_002 primer | TAGTACACGGCGCTGTCC |
| 146 | AB_015 primer | CGTGAGCCCTgcgGATTATTGGGGC |
| 147 | AB_016 primer | TAGGAGCCGTAGGTGGCA |
| 148 | AB_037 primer | CTGTGCCACCgtCGGCTCCTACG |
| 149 | AB_038 primer | TAGTACACGGCGCTGTCC |
| 150 | AB_039 primer | CTGTGCCACCctCGGCTCCTAC |
| 151 | AB_040 primer | CTGTGCCACCatCGGCTCCTAC |
| 152 | AB_042 primer | CTGTGCCACCatgGGCTCCTACGTGAGCCC |
| 153 | AB_086 primer | CCGCTCGGAGgcCACCACCGAG |
| 154 | AB_087 primer | GCGCAGTAGTACACGGCC |
| 155 | AB_088 primer | CTCGGAGATCgCCACCGAGTTC |
| 156 | AB_089 primer | CGGGCGCAGTAGTACACG |
| 157 | AB_117 primer | GCGCAGTAGTACACGGCC |
| 158 | AB_120 primer | CCGCTCGGAGggcACCACCGAGTTC |
| 159 | AB_123 primer | CCGCTCGGAGttcACCACCGAGT |
| 160 | AB_125 primer | CCGCTCGGAGtggACCACCGAGTTC |
| 161 | EF1α promoter | GGCTCCGGTGCCCGTCAGTGGGCAGAGCGCACATC |
| | | GCCCACAGTCCCCGAGAAGTTGGGGGGAGGGGTCG |
| | | GCAATTGAACCGGTGCCTAGAGAAGGTGGCGCGGG |
| | | GTAAACTGGGAAAGTGATGTCGTGTACTGGCTCCG |
| | | CCTTTTTCCCGAGGGTGGGGGAGAACCGTATATAA |
| | | GTGCAGTAGTCGCCGTGAACGTTCTTTTTCGCAACG |
| | | GGTTTGCCGCCAGAACACAGGTAAGTGCCGTGTGT |
| | | GGTTCCCGCGGGCCTGGCCTCTTTACGGGTTATGGC |
| | | CCTTGCGTGCCTTGAATTACTTCCACCTGGCTGCAG |
| | | TACGTGATTCTTGATCCCGAGCTTCGGGTTGGAAGT |
| | | GGGTGGGAGAGTTCGAGGCCTTGCGCTTAAGGAGC |
| | | CCCTTCGCCTCGTGCTTGAGTTGAGGCCTGGCCTGG |
| | | GCGCTGGGGCCGCCGCGTGCGAATCTGGTGGCACC |
| | | TTCGCGCCTGTCTCGCTGCTTTCGATAAGTCTCTAG |
| | | CCATTTAAAATTTTTGATGACCTGCTGCGACGCTTT |
| | | TTTTCTGGCAAGATAGTCTTGTAAATGCGGGCCAAG |
| | | ATCTGCACACTGGTATTTCGGTTTTTGGGGCCGCGG |
| | | GCGGCGACGGGGCCCGTGCGTCCCAGCGCACATGT |
| | | TCGGCGAGGCGGGGCCTGCGAGCGCGGCCACCGAG |
| | | AATCGGACGGGGGTAGTCTCAAGCTGGCCGGCCTG |
| | | CTCTGGTGCCTGGTCTCGCGCCGCCGTGTATCGCCC |
| | | CGCCCTGGGCGGCAAGGCTGGCCCGGTCGGCACCA |
| | | GTTGCGTGAGCGGAAAGATGGCCGCTTCCCGGCCC |
| | | TGCTGCAGGGAGCTCAAAATGGAGGACGCGGCGCT |
| | | CGGGAGAGCGGGCGGGTGAGTCACCCACACAAAGG |
| | | AAAAGGGCCTTTCCGTCCTCAGCCGTCGCTTCATGT |
| | | GACTCCACGGAGTACCGGGCGCCGTCCAGGCACCT |
| | | CGATTAGTTCTCGAGCTTTTGGAGTACGTCGTCTTT |
| | | AGGTTGGGGGGAGGGGTTTTATGCGATGGAGTTTC |
| | | CCCACACTGAGTGGGTGGAGACTGAAGTTAGGCCA |
| | | GCTTGGCACTTGATGTAATTCTCCTTGGAATTTGCC |
| | | CTTTTTGAGTTTGGATCTTGGTTCATTCTCAAGCCTC |
| | | AGACAGTGGTTCAAAGTTTTTTTCTTCCATTTCAGG |
| | | TGTCGTGA |
| 162 | CD8 alpha chain | MALPVTALLLPLALLLHAARP |
| | signal peptide | |
| 163 | CD8 alpha chain | ATGGCCTTACCAGTGACCGCCTTGCTCCTGCCGCTG |
| | signal peptide | GCCTTGCTGCTCCACGCCGCCAGGCCG |

*CDR regions underlined and italicized. CDR1, CD2, and CD3 regions are underlined and italicized in the VH and VL sequences. Only HCDR3 regions are underlined and italicized in the scFv and CAR sequences. Mutated amino acids of nucleotides in the HCDR3 regions are bolded.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A chimeric antigen receptor (CAR), comprising:
(a) an extracellular binding domain;
(b) a costimulatory domain; and
(c) an activation domain,
wherein the extracellular binding domain comprises a heavy chain variable (V_{H}) region comprising complementary determining 1 (HC CDR1), complementary determining 2 (HC CDR2), and complementary determining 3 (HC CDR3) regions; and a light chain variable (V_{L}) region comprising complementary determining 1 (LC CDR1), complementary determining 2 (LC CDR2), and complementary determining 3 (LC CDR3) regions, wherein:
(i) the V_{H} region comprises HC CDR1 and HC CDR2 regions comprising amino acid sequences set forth SEQ ID NOs: 1 and 2, respectively, and an HC CDR3 region comprising an amino acid sequence set forth in any one of SEQ ID NOs: 3-8; and the V_{L} region comprises LC CDR1, LC CDR2, and LC CDR3 regions comprising amino acid sequences set forth in SEQ ID NOs: 9-11, respectively, or
(ii) the V_{H} region comprises HC CDR1 and HC CDR2 regions comprising amino acid sequences set forth in SEQ ID NOs: 70 and 71, respectively, and an HC CDR3 region comprising an amino acid sequence set forth in any one of SEQ ID NOs: 72-75; and the V_{L} region comprises LC CDR1, LC CDR2, and LC CDR3 regions comprising amino acid sequences set forth in SEQ ID NOs: 76-78, respectively.

2. The CAR of claim 1, wherein the V_{H} region comprises the amino acid sequences set forth in (i).

3. The CAR of claim 1, wherein the HC CDR3 comprises an amino acid sequence set forth in SEQ ID NO: 5 or SEQ ID NO: 8.

4. The CAR of claim 1, which comprises V_{H} and V_{L} region amino acid sequences selected from the group consisting of:
(a) SEQ ID NOs: 13 and 19, respectively;
(b) SEQ ID NOs: 14 and 19, respectively;
(c) SEQ ID NOs: 15 and 19, respectively;
(d) SEQ ID NOs: 16 and 19, respectively;
(e) SEQ ID NOs: 17 and 19, respectively; and
(f) SEQ ID NOs: 18 and 19, respectively;

5. The CAR of claim 1, wherein the extracellular domain is a single chain antibody fragment (scFv).

6. The CAR of claim 5, wherein scFv comprises a (GxS)n linker between the V_{H} and V_{L} regions.

7. The CAR of claim 5, wherein the scFv comprises an amino acid sequence set forth in any one of SEQ ID NOs: 29-34.

8. The CAR of claim 1, wherein the V_{H} region comprises the amino acid sequences set forth in (ii).

9. The CAR of claim 8, wherein the HC CDR3 comprises an amino acid sequence set forth in SEQ ID NO: 74 or 75.

10. The CAR of claim 8, which comprises VH and VL region amino acid sequences selected from the group consisting of:
(a) SEQ ID NOs: 80 and 84, respectively;
(b) SEQ ID NOs: 81 and 84, respectively;
(c) SEQ ID NOs: 82 and 84, respectively; and
(d) SEQ ID NOs: 83 and 84, respectively.

11. The CAR of claim 8, wherein the extracellular domain is an scFv.

12. The CAR of claim 11, wherein the scFv comprises a (GxS)ₙ linker between the V_{H} and V_{L} regions.

13. The CAR of claim 11, wherein the scFv comprises an amino acid sequence set forth in any one of SEQ ID NOs: 92-95.

14. The CAR of claim 1, wherein the costimulatory domain is from 4-1BB (CD 137), CD28, OX40, ICOS, GITR, CD27, CD30, CD40, DAP 10, DAP12, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD 160, B7-H3, a ligand that specifically binds with CD83 or a combination thereof; optionally wherein the costimulatory domain comprises an amino acid sequence set forth in any one of SEQ ID NOs: 122-124.

15. The CAR of claim 14, wherein the costimulatory domain comprises 4-1BB or CD28.

16. The CAR of claim 1, wherein the extracellular binding domain comprises costimulatory domains from 4-1BB and CD28, or CD-28 and OX-40.

17. The CAR of claim 1, wherein the activation domain is from CD3 zeta, common FcR gamma (FCER1G), Fc gamma RIIa, FcR beta (Fc epsilon lb), CD3 gamma, CD3 delta, CD3epsilon, CD5, CD22, CD79a, CD79b, CD278 (ICOS), FcεRI, or CD66d.

18. The CAR of claim 17, wherein the activation domain is from CD3 zeta, optionally comprising the amino acid sequence of SEQ ID NO: 125.

19. The CAR of claim 1, further comprising a hinge domain, a transmembrane domain, or a combination thereof, which optionally is located between the extracellular binding domain and the costimulatory domain.

20. The CAR of claim 19 comprising a hinge domain from CD8 alpha, CD28, or IgG4; optionally wherein the hinge domain comprises an amino sequence set forth in any one of SEQ ID NOs: 126-128.

21. The CAR of claim 19, comprising a transmembrane domain from a cell surface receptor selected from the group consisting of an alpha, beta or zeta chain of a T cell receptor, CD8 alpha, CD28, ICOS, GITR, CD3 epsilon, CD45, CD4, CD5, CD8, CD9, CD16, CD22, CD33, CD37, CD64, CD80, CD86, CD134, CD137, CD154, CD271, TNFRSF19, Killer Cell Immunoglobulin-Like Receptor (KIR), and a combination thereof.

22. The CAR of claim 21, wherein the transmembrane domain is from CD8 alpha, CD28, ICOS, and GITR; optionally wherein the transmembrane domain comprises an amino acid sequence set forth in any one SEQ ID NOs: 129-131.

23. The CAR of claim 1, further comprising a signal peptide located at the N-terminus of a CAR precursor protein; optionally wherein the signal peptide is a CD8 alpha chain signal peptide.

24. The CAR of claim 1, further comprising a second extracellular antigen binding domain.

25. The CAR of claim 23, wherein the second extracellular antigen binding domain comprises a second scFv.

26. The CAR of claim 24, wherein the second extracellular antigen binding domain comprises an ICAM-1 binding domain.

27. The CAR of claim 26, wherein the ICAM-1 binding domain comprises an αL subunit I domain of human lymphocyte function-associated antigen-1 (LFA-1); optionally wherein the ICAM-1 binding domain comprises an αL subunit I domain of human lymphocyte function-associated antigen-1 (LFA-1).

28. The CAR of claim 1, wherein the CAR comprises an amino acid sequence set forth in any one of SEQ ID NOs: 43-48 or 102-105.

29-36. (canceled)

37. A population of immune cells expressing the CAR of claim 1.

38. (canceled)

39. The population of immune cells of claim 37, wherein the immune cells are T cells, natural killer (NK) cells, tumor infiltrating lymphocytes, dendritic cells, macrophages, B cells, neutrophils, eosinophils, basophils, mast cells, myeloid-derived suppressor cells, stem cells, precursors thereof, subtypes thereof, or a combination thereof; optionally wherein the immune cell is a human immune cell.

40. The population of immune cells of claim 39, wherein the immune cells are T cells.

41. The population of immune cells of claim 40, wherein the T cells express SSTR2.

42. The population of immune cells of claim 37, further comprising a second population of immune cells, wherein the second population of immune cells expresses a second CAR or another polypeptide of interest.

43. (canceled)

44. The population of immune cells of claim 42, wherein the second CAR comprises an ICAM-1 binding domain; optionally wherein the ICAM-1 binding domain comprises an αL subunit I domain of human LFA-1.

45. A cell therapy-based method of treating cancer, comprising administering to a subject in need thereof the population of immune cells of claim 37.

46-66. (canceled)

67. A method for treating cancer and monitoring CAR-T cell distribution in a patient, comprising:
incubating the population of CAR-T cells of claim 41 with a radioactive label that binds to SSTR2,
intravenously infusing the labeled CAR-T cells into a patient in an amount of 10⁴-10⁸ cells/kg patient, and
detecting the labeled CAR-T cell distribution by PET/CT imaging,
wherein the labeled CAR-T cells are infiltrated into cancer cells to kill the cancer cells.

68-70. (canceled)

71. A method for treating cancer and monitoring CAR-T cell distribution in a patient, comprising:
intravenously infusing the population of CAR-T cells of claim 41 into a patient, wherein the CAR-T cells express or have been transduced to express at least 100,000 molecules of SSTR2 per T cell
injecting into the patient a radioactive label that binds to SSTR2 at least one hour prior to PET/CT imaging, and
detecting the labeled CAR-T cell distribution by PET/CT imaging,
wherein the labeled CAR-T cells are infiltrated into cancer cells to kill the cancer cells.

72. (canceled)

73. A method of producing a population of genetically engineered immune cells, the method comprising:
(a) providing a population of immune cells; and
(b) introducing into the immune cells a nucleic acid coding for the CAR claim 1 to produce a population of genetically engineered immune cells.

74-78. (canceled)


SEQUENCE LISTING (163 sequences)

SEQ ID NO: 1 - protein, 8 aa, synthetic construct
SGYTFTSY

SEQ ID NO: 2 - protein, 17 aa, synthetic construct
LEWIGMIDPSNSDTRFN

SEQ ID NO: 3 - protein, 10 aa, synthetic construct
AGSYVSPLDY

SEQ ID NO: 4 - protein, 10 aa, synthetic construct
YGSYVSPADY

SEQ ID NO: 5 - protein, 10 aa, synthetic construct
VGSYVSPLDY

SEQ ID NO: 6 - protein, 10 aa, synthetic construct
LGSYVSPLDY

SEQ ID NO: 7 - protein, 10 aa, synthetic construct
IGSYVSPLDY

SEQ ID NO: 8 - protein, 10 aa, synthetic construct
MGSYVSPLDY

SEQ ID NO: 9 - protein, 11 aa, synthetic construct
QSLLYTSSQKN

SEQ ID NO: 10 - protein, 12 aa, synthetic construct
PKLLIYWASTRE

SEQ ID NO: 11 - protein, 5 aa, synthetic construct
YYAYP

SEQ ID NO: 12 - protein, 119 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSS

SEQ ID NO: 13 - protein, 119 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATAGSYVSPLDYWGQGTSVTVSS

SEQ ID NO: 14 - protein, 119 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPADYWGQGTSVTVSS

SEQ ID NO: 15 - protein, 119 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATVGSYVSPLDYWGQGTSVTVSS

SEQ ID NO: 16 - protein, 119 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATLGSYVSPLDYWGQGTSVTVSS

SEQ ID NO: 17 - protein, 119 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATIGSYVSPLDYWGQGTSVTVSS

SEQ ID NO: 18 - protein, 119 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATMGSYVSPLDYWGQGTSVTVSS

SEQ ID NO: 19 - protein, 114 aa, synthetic construct
DIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKR

SEQ ID NO: 20 - DNA, 357 bp, synthetic construct
  source 1..357: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccacctacggctcctacgtgagccctttggattattggggccagggcacctccgtcaccgtgtcctcc

SEQ ID NO: 21 - DNA, 357 bp, synthetic construct
  source 1..357: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccgcgggctcctacgtgagccctttggattattggggccagggcacctccgtcaccgtgtcctcc

SEQ ID NO: 22 - DNA, 357 bp, synthetic construct
  source 1..357: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccacctacggctcctacgtgagccctgcggattattggggccagggcacctccgtcaccgtgtcctcc

SEQ ID NO: 23 - DNA, 357 bp, synthetic construct
  source 1..357: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccgtcggctcctacgtgagccctttggattattggggccagggcacctccgtcaccgtgtcctcc

SEQ ID NO: 24 - DNA, 357 bp, synthetic construct
  source 1..357: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccctcggctcctacgtgagccctttggattattggggccagggcacctccgtcaccgtgtcctcc

SEQ ID NO: 25 - DNA, 357 bp, synthetic construct
  source 1..357: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccatcggctcctacgtgagccctttggattattggggccagggcacctccgtcaccgtgtcctcc

SEQ ID NO: 26 - DNA, 357 bp, synthetic construct
  source 1..357: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccatgggctcctacgtgagccctttggattattggggccagggcacctccgtcaccgtgtcctcc

SEQ ID NO: 27 - DNA, 342 bp, synthetic construct
  source 1..342: mol_type = other DNA
gacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgc

SEQ ID NO: 28 - protein, 248 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKR

SEQ ID NO: 29 - protein, 248 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATAGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKR

SEQ ID NO: 30 - protein, 248 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPADYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKR

SEQ ID NO: 31 - protein, 248 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATVGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKR

SEQ ID NO: 32 - protein, 248 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATLGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKR

SEQ ID NO: 33 - protein, 248 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATIGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKR

SEQ ID NO: 34 - protein, 248 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATMGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKR

SEQ ID NO: 35 - DNA, 744 bp, synthetic construct
  source 1..744: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccacctacggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgc

SEQ ID NO: 36 - DNA, 744 bp, synthetic construct
  source 1..744: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccgcgggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgc

SEQ ID NO: 37 - DNA, 744 bp, synthetic construct
  source 1..744: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccacctacggctcctacgtgagccctgcggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgc

SEQ ID NO: 38 - DNA, 744 bp, synthetic construct
  source 1..744: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccgtcggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgc

SEQ ID NO: 39 - DNA, 744 bp, synthetic construct
  source 1..744: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccctcggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgc

SEQ ID NO: 40 - DNA, 744 bp, synthetic construct
  source 1..744: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccatcggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgc

SEQ ID NO: 41 - DNA, 744 bp, synthetic construct
  source 1..744: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccatgggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgc

SEQ ID NO: 42 - protein, 476 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 476 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATAGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 476 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPADYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 476 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATVGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 476 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATLGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 476 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATIGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 476 aa, synthetic construct
QVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATMGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 885 aa, synthetic construct
GSEQKLISEEDLGSQVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 50 - protein, 885 aa, synthetic construct
GSEQKLISEEDLGSQVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATAGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 51 - protein, 885 aa, synthetic construct
GSEQKLISEEDLGSQVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATYGSYVSPADYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 52 - protein, 885 aa, synthetic construct
GSEQKLISEEDLGSQVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATVGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 53 - protein, 885 aa, synthetic construct
GSEQKLISEEDLGSQVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATLGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 54 - protein, 885 aa, synthetic construct
GSEQKLISEEDLGSQVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATIGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 55 - protein, 885 aa, synthetic construct
GSEQKLISEEDLGSQVQLQQSGPELVRPGASVKMSCRASGYTFTSYWLHWVKQRPGQGLEWIGMIDPSNSDTRFNPNFKDKATLNVDRSSNTAYMLLSSLTSADSAVYYCATMGSYVSPLDYWGQGTSVTVSSGGGGSGGGGSGGGGSDIMMSQSPSSLTVSVGEKVTVSCKSSQSLLYTSSQKNYLAWYQQKPGQSPKLLIYWASTRESGVPDRFTGSGSGTDFTLTITSVKADDLAVYYCQQYYAYPWTFGGGTKLEIKRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 56 - DNA, 1428 bp, synthetic construct
  source 1..1428: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccacctacggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 57 - DNA, 1428 bp, synthetic construct
  source 1..1428: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccgcgggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 58 - DNA, 1428 bp, synthetic construct
  source 1..1428: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccacctacggctcctacgtgagccctgcggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 59 - DNA, 1428 bp, synthetic construct
  source 1..1428: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccgtcggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 60 - DNA, 1428 bp, synthetic construct
  source 1..1428: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccctcggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 61 - DNA, 1428 bp, synthetic construct
  source 1..1428: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccatcggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 62 - DNA, 1428 bp, synthetic construct
  source 1..1428: mol_type = other DNA
caggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccatgggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 63 - DNA, 3930 bp, synthetic construct
  source 1..3930: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccacctacggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 64 - DNA, 3930 bp, synthetic construct
  source 1..3930: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccgcgggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 65 - DNA, 3930 bp, synthetic construct
  source 1..3930: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccacctacggctcctacgtgagccctgcggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 66 - DNA, 3930 bp, synthetic construct
  source 1..3930: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccgtcggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 67 - DNA, 3930 bp, synthetic construct
  source 1..3930: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccctcggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 68 - DNA, 3930 bp, synthetic construct
  source 1..3930: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccatcggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 69 - DNA, 3930 bp, synthetic construct
  source 1..3930: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctccagcagagcggccctgagctggtgcgccccggcgcttccgtgaagatgtcatgccgagcttctggctacacgttcaccagctattggctacactgggtcaagcagaggcccggacagggtctggagtggatcggtatgattgacccgtcaaatagtgacacccgcttcaaccccaacttcaaggacaaggccactctcaacgtggaccgctcgagcaacacggcctacatgctgctgtcctctctgacctcggcggacagcgccgtgtactactgtgccaccatgggctcctacgtgagccctttggattattggggccagggcacctccgtcaccgtgtcctccggtggaggcggttcaggcggaggtggctctggcggtggcggatcggacatcatgatgtctcagagtccctcttctctgaccgtgtccgtgggcgagaaggtgaccgtgtcatgcaagtcctcacagagccttctgtacaccagctcccagaagaactacctggcgtggtaccagcagaagcccggacagagccccaagctgctgatctattgggcttccacccgcgagagcggcgtccccgaccgcttcaccggctccggctccgggaccgacttcaccctgaccatcacctccgtgaaggccgatgacctggccgtgtactactgtcaacagtattacgcctacccgtggaccttcggaggcggcaccaagctggagatcaagcgcgctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 70 - protein, 7 aa, synthetic construct
GYIFTAY

SEQ ID NO: 71 - protein, 16 aa, synthetic construct
LEWMGWIKPNNGLANY

SEQ ID NO: 72 - protein, 9 aa, synthetic construct
SEATTEFDY

SEQ ID NO: 73 - protein, 9 aa, synthetic construct
SEWTTEFDY

SEQ ID NO: 74 - protein, 9 aa, synthetic construct
SEFTTEFDY

SEQ ID NO: 75 - protein, 9 aa, synthetic construct
SEGTTEFDY

SEQ ID NO: 76 - protein, 6 aa, synthetic construct
ESVDSY

SEQ ID NO: 77 - protein, 12 aa, synthetic construct
PKLLIYRASTRE

SEQ ID NO: 78 - protein, 6 aa, synthetic construct
SKEDPL

SEQ ID NO: 79 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 80 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEATTEFDYWGQGTLVTVSS

SEQ ID NO: 81 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEWTTEFDYWGQGTLVTVSS

SEQ ID NO: 82 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEFTTEFDYWGQGTLVTVSS

SEQ ID NO: 83 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEGTTEFDYWGQGTLVTVSS

SEQ ID NO: 84 - protein, 111 aa, synthetic construct
DIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIK

SEQ ID NO: 85 - DNA, 354 bp, synthetic construct
  source 1..354: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagatcaccaccgagttcgactattggggccagggcaccctggtgaccgtgtcgtcc

SEQ ID NO: 86 - DNA, 354 bp, synthetic construct
  source 1..354: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggaggccaccaccgagttcgactattggggccagggcaccctggtgaccgtgtcgtcc

SEQ ID NO: 87 - DNA, 354 bp, synthetic construct
  source 1..354: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagtggaccaccgagttcgactattggggccagggcaccctggtgaccgtgtcgtcc

SEQ ID NO: 88 - DNA, 354 bp, synthetic construct
  source 1..354: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagttcaccaccgagttcgactattggggccagggcaccctggtgaccgtgtcgtcc

SEQ ID NO: 89 - DNA, 354 bp, synthetic construct
  source 1..354: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagggcaccaccgagttcgactattggggccagggcaccctggtgaccgtgtcgtcc

SEQ ID NO: 90 - DNA, 333 bp, synthetic construct
  source 1..333: mol_type = other DNA
gacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaag

SEQ ID NO: 91 - protein, 244 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIK

SEQ ID NO: 92 - protein, 244 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEATTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIK

SEQ ID NO: 93 - protein, 244 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEWTTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIK

SEQ ID NO: 94 - protein, 244 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEFTTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIK

SEQ ID NO: 95 - protein, 244 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEGTTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIK

SEQ ID NO: 96 - DNA, 732 bp, synthetic construct
  source 1..732: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagatcaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaag

SEQ ID NO: 97 - DNA, 732 bp, synthetic construct
  source 1..732: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggaggccaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaag

SEQ ID NO: 98 - DNA, 732 bp, synthetic construct
  source 1..732: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagtggaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaag

SEQ ID NO: 99 - DNA, 732 bp, synthetic construct
  source 1..732: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagttcaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaag

SEQ ID NO: 100 - DNA, 732 bp, synthetic construct
  source 1..732: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagggcaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaag

SEQ ID NO: 101 - protein, 472 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 472 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEATTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 472 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEWTTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 472 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEFTTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 472 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEGTTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 881 aa, synthetic construct
GSEQKLISEEDLGSQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 107 - protein, 881 aa, synthetic construct
GSEQKLISEEDLGSQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEATTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 108 - protein, 881 aa, synthetic construct
GSEQKLISEEDLGSQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEWTTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 109 - protein, 881 aa, synthetic construct
GSEQKLISEEDLGSQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEFTTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 110 - protein, 881 aa, synthetic construct
GSEQKLISEEDLGSQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEGTTEFDYWGQGTLVTVSSGGGGSGGGGSGGGGSDIVLTQSPDSLAVSLGERATINCKSSESVDSYANSFMHWYQQKPGQPPKLLIYRASTRESGVPDRFSGSGSRTDFTLTISSLQAEDVAVYYCQQSKEDPLTFGGGTKVEIKASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRTSGSGATNFSLLKQAGDVEENPGPSRMDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 111 - DNA, 1416 bp, synthetic construct
  source 1..1416: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagatcaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaaggctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 112 - DNA, 1416 bp, synthetic construct
  source 1..1416: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggaggccaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaaggctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 113 - DNA, 1416 bp, synthetic construct
  source 1..1416: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagtggaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaaggctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 114 - DNA, 1416 bp, synthetic construct
  source 1..1416: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagttcaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaaggctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 115 - DNA, 1416 bp, synthetic construct
  source 1..1416: mol_type = other DNA
caggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagggcaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaaggctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 116 - DNA, 3918 bp, synthetic construct
  source 1..3918: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagatcaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaaggctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 117 - DNA, 3918 bp, synthetic construct
  source 1..3918: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggaggccaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaaggctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 118 - DNA, 3918 bp, synthetic construct
  source 1..3918: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagtggaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaaggctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 119 - DNA, 3918 bp, synthetic construct
  source 1..3918: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagttcaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaaggctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 120 - DNA, 3918 bp, synthetic construct
  source 1..3918: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgaacaaaaactcatctcagaagaggatctgggatcgcaggtgcagctcgtgcagagcggcgccgaggtgaagaagcccggcgcttccgtgaaggtgtcctgtaaggcctctggctacatcttcaccgcctacaccatgcactgggtgcgccaggccccgggacaggggctggagtggatgggctggatcaagcctaacaacggtctggccaactacgcgcagaagttccagggccgcgtgaccatgactcgggacaccagcatctctaccgcgtacatggagctgtcccgcctgcgctccgatgacacggccgtgtactactgcgcccgctcggagggcaccaccgagttcgactattggggccagggcaccctggtgaccgtgtcgtccggtggaggcggttcaggcggaggtggctctggcggtggcggatcggacatcgtgctgacccagagccctgacagcctggccgtgtccctgggcgagcgcgccaccattaactgcaagagctccgagagtgtcgatagctacgccaactccttcatgcactggtaccagcagaagcccggccagccacccaagctgctgatctacagggcttccacccgcgagagcggcgtccccgacaggttttcaggttctggctcccgcaccgacttcaccctgaccatctcttctctgcaggccgaggatgtggcggtgtactactgtcaacagtccaaggaggacccgcttaccttcggcggcggcaccaaggtggagatcaaggctagcacgaccactccggcgccgcgcccaccgactccggccccaactatcgcgagccagcccctgtcgctgaggccggaagcatgccgccctgccgccggaggtgctgtgcatacccggggattggacttcgcatgcgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcactagtggaagcggagctactaacttcagcctgctgaagcaggctggagacgtggaggagaaccctggaccttctagaatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 121 - protein, 15 aa, synthetic construct
GGGGSGGGGSGGGGS

SEQ ID NO: 122 - protein, 42 aa, synthetic construct
KRGRKKLLYIFKQPFMRPVQTTQEEDGCSCRFPEEEEGGCEL

SEQ ID NO: 123 - protein, 41 aa, synthetic construct
RSKRSRLLHSDYMNMTPRRPGPTRKHYQPYAPPRDFAAYRS

SEQ ID NO: 124 - protein, 42 aa, synthetic construct
ALYLLRRDQRLPPDAHKPPGGGSFRTPIQEEQADAHSTLAKI

SEQ ID NO: 125 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 45 aa, synthetic construct
TTTPAPRPPTPAPTIASQPLSLRPEACRPAAGGAVHTRGLDFACD

SEQ ID NO: 127 - protein, 47 aa, synthetic construct
RAAAIEVMYPPPYLDNEKSNGTIIHVKGKHLCPSPLFPGPSKPKDPK

SEQ ID NO: 128 - protein, 11 aa, synthetic construct
SKYGPPCPSCP

SEQ ID NO: 129 - protein, 24 aa, synthetic construct
IYIWAPLAGTCGVLLLSLVITLYC

SEQ ID NO: 130 - protein, 24 aa, synthetic construct
FWVLVVVGGVLACYSLLVTVAFII

SEQ ID NO: 131 - protein, 24 aa, synthetic construct
FWLPIGCAAFVVVCILGCILICWL

SEQ ID NO: 132 - protein, 25 aa, synthetic construct
LGWLTVVLLAVAACVLLLTSAQLGL

SEQ ID NO: 133 - protein, 1145 aa, Homo sapiens
YNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLYPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 134 - protein, 181 aa, Homo sapiens
VDLVFLFDGSMSLQPDEFQKILDFMKDVMKKLSNTSYQFAAVQFSTSYKTEFDFSDYVKWKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIE

SEQ ID NO: 135 - protein, 369 aa, Homo sapiens
MDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAFLSDNFKKSFQNVLCLVKVSGTDDGERSDSKQDKSRLNETTETQRTLLNGDLQTSI

SEQ ID NO: 136 - protein, 314 aa, Homo sapiens
MDMADEPLNGSHTWLSIPFDLNGSVVSTNTSNQTEPYYDLTSNAVLTFIYFVVCIIGLCGNTLVIYVILRYAKMKTITNIYILNLAIADELFMLGLPFLAMQVALVHWPFGKAICRVVMTVDGINQFTSIFCLTVMSIDRYLAVVHPIKSAKWRRPRTAKMITMAVWGVSLLVILPIMIYAGLRSNQWGRSSCTINWPGESGAWYTGFIIYTFILGFLVPLTIICLCYLFIIIKVKSSGIRVGSSKRKKSEKKVTRMVSIVVAVFIFCWLPFYIFNVSSVSMAISPTPALKGMFDFVVVLTYANSCANPILYAF

SEQ ID NO: 137 - DNA, 1110 bp, Homo sapiens
  source 1..1110: mol_type = genomic DNA
atggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctaa

SEQ ID NO: 138 - DNA, 936 bp, Homo sapiens
  source 1..936: mol_type = genomic DNA
atggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatat

SEQ ID NO: 139 - protein, 22 aa, synthetic construct
GSGATNFSLLKQAGDVEENPGP

SEQ ID NO: 140 - protein, 23 aa, synthetic construct
GSGQCTNYALLKLAGDVESNPGP

SEQ ID NO: 141 - protein, 21 aa, synthetic construct
GSGEGRGSLLTCGDVEENPGP

SEQ ID NO: 142 - protein, 25 aa, synthetic construct
GSGVKQTLNFDLLKLAGDVESNPGP

SEQ ID NO: 143 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 144 - DNA, 28 bp, synthetic construct
  source 1..28: mol_type = other DNA
ctgtgccaccgcgggctcctacgtgagc

SEQ ID NO: 145 - DNA, 18 bp, synthetic construct
  source 1..18: mol_type = other DNA
tagtacacggcgctgtcc

SEQ ID NO: 146 - DNA, 25 bp, synthetic construct
  source 1..25: mol_type = other DNA
cgtgagccctgcggattattggggc

SEQ ID NO: 147 - DNA, 18 bp, synthetic construct
  source 1..18: mol_type = other DNA
taggagccgtaggtggca

SEQ ID NO: 148 - DNA, 23 bp, synthetic construct
  source 1..23: mol_type = other DNA
ctgtgccaccgtcggctcctacg

SEQ ID NO: 149 - DNA, 18 bp, synthetic construct
  source 1..18: mol_type = other DNA
tagtacacggcgctgtcc

SEQ ID NO: 150 - DNA, 22 bp, synthetic construct
  source 1..22: mol_type = other DNA
ctgtgccaccctcggctcctac

SEQ ID NO: 151 - DNA, 22 bp, synthetic construct
  source 1..22: mol_type = other DNA
ctgtgccaccatcggctcctac

SEQ ID NO: 152 - DNA, 30 bp, synthetic construct
  source 1..30: mol_type = other DNA
ctgtgccaccatgggctcctacgtgagccc

SEQ ID NO: 153 - DNA, 22 bp, synthetic construct
  source 1..22: mol_type = other DNA
ccgctcggaggccaccaccgag

SEQ ID NO: 154 - DNA, 18 bp, synthetic construct
  source 1..18: mol_type = other DNA
gcgcagtagtacacggcc

SEQ ID NO: 155 - DNA, 22 bp, synthetic construct
  source 1..22: mol_type = other DNA
ctcggagatcgccaccgagttc

SEQ ID NO: 156 - DNA, 18 bp, synthetic construct
  source 1..18: mol_type = other DNA
cgggcgcagtagtacacg

SEQ ID NO: 157 - DNA, 18 bp, synthetic construct
  source 1..18: mol_type = other DNA
gcgcagtagtacacggcc

SEQ ID NO: 158 - DNA, 25 bp, synthetic construct
  source 1..25: mol_type = other DNA
ccgctcggagggcaccaccgagttc

SEQ ID NO: 159 - DNA, 23 bp, synthetic construct
  source 1..23: mol_type = other DNA
ccgctcggagttcaccaccgagt

SEQ ID NO: 160 - DNA, 25 bp, synthetic construct
  source 1..25: mol_type = other DNA
ccgctcggagtggaccaccgagttc

SEQ ID NO: 161 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 162 - protein, 21 aa, synthetic construct
MALPVTALLLPLALLLHAARP

SEQ ID NO: 163 - DNA, 63 bp, synthetic construct
  source 1..63: mol_type = other DNA
atggccttaccagtgaccgccttgctcctgccgctggccttgctgctccacgccgccaggccg